(19) United States
(12) Patent Application Publication
(10) Pub. No.: US 2006/0223875 A1
(43) Pub. Date: Oct. 5, 2006

(54) Methods and compositions for production, formulation and use of 1-aryl-3-azabicyclo[3.1.0]hexanes

(76) Inventors: Phil Skolnick, Edgewater, NJ (US); Anthony Basile, Hoboken, NJ (US); Zhengming Chen, Belle Mead, NJ (US)

Correspondence Address: BLACK LOWE & GRAHAM PLLC, Suite 4800, 701 Fifth Avenue, Seattle, WA, 98104, US

(21) Appl. No.: 11/371,178

(22) Filed: Mar. 7, 2006

Related U.S. Application Data
(60) Provisional application No. 60/661,662, filed on Mar. 8, 2005.
(60) Provisional application No. 60/701,562, filed on Jul. 22, 2005.

(51) Int. Cl. A61K 31/403; C07D 209/00

(52) U.S. Cl. 514/412; 548/452

(57) ABSTRACT

The invention provides novel 1-aryl-3-azabicyclo[3.1.0]hexanes that are active for modulating biogenic amine transport, along with compositions and methods for using these compounds to treat central nervous system disorders. Certain 1-aryl-3-azabicyclo[3.1.0]hexanes are provided that have at least one substituent on the aryl ring. In other embodiments 1-aryl-3-azabicyclo[3.1.0]hexanes are provided that have a substitution on the nitrogen at the ‘3’ position. In additional embodiments 1-aryl-3-azabicyclo[3.1.0]hexanes are provided which have one substitution on the aryl ring, as well as a substitution on the nitrogen at the ‘3’ position. The invention also provides novel methods of making aryl- and aza-substituted 1-aryl-3-azabicyclo[3.1.0]hexanes, including synthetic methods that form novel intermediate compounds of the invention for producing aryl- and aza-substituted 1-aryl-3-azabicyclo[3.1.0]hexanes.

### REFERENCE TO RELATED APPLICATIONS

This application is related to and claims priority from U.S. Provisional Application 60/661,662, filed on Mar. 8, 2005 and 60/701,562 filed on Jul. 22, 2005, the disclosures of which Provisional Applications are incorporated herein by reference in their entirety.

### TECHNICAL FIELD

The present invention relates to novel 1-aryl-3-azabicyclo[3.1.0]hexanes, intermediates for the production thereof and methods for preparing, formulating, and using 1-aryl-3-azabicyclo[3.1.0]hexanes.

### BACKGROUND OF THE INVENTION

A series of 1-aryl-3-azabicyclo[3.1.0]hexanes was previously synthesized, and among these compounds, some candidates were reported to have analgesic properties (Epstein et al., J. Med. Chem. 24:481-90, 1981; U.S. Pat. No. 4,131,611 issued Dec. 26, 1978 to Fanshawe et al.). Within the limited series of 1-aryl-3-azabicyclo[3.1.0]hexanes heretofore produced and characterized, bicifadine hydrochloride (the hydrochloric acid salt of (±)-1-(4-methylphenyl-3-azabicyclo[3.1.0]-hexane; Formula I, below) was reported to have the most potent, non-narcotic analgesic activity (Id.; see also, Wang et al., J. Clin. Pharmacol. 22:160-4, 1982). The analgesic efficacy of orally administered 75 and 150 mg bicifadine hydrochloride was compared to 650 mg aspirin and placebo in a double-blind, single-dose study. Significant analgesic activity was reported with 650 mg aspirin and 150 mg bicifadine compared to placebo, and side effects were reported to be minor. Based on additional studies in dental surgery patients, bicifadine can reportedly produce analgesia comparable to the narcotic, codeine and the narcotic-like agent tramadol, respectively (Czobor P., et al., 2003); (Czobor P., et al., 2004).

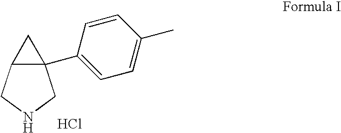

Certain other aryl substituted 3-azabicyclo[3.1.0]hexanes have been reported to inhibit transport (e.g., reuptake) of norepinephrine, serotonin, and/or dopamine. For example, 1-(3,4-dichlorophenyl)-3-azabicyclo[3.1.0]hexane hydrochloride was reported to inhibit reuptake of all three of these biogenic amines, norepinephrine, serotonin, and dopamine (Skolnick, P., et al., Life Sci., 73: 3175-3179, 2003; Beer et al., J. Clin. Pharmacol. 44:1360-1367, 2004). Based on this observed activity involving reuptake inhibition of norepinephrine, serotonin, and dopamine, 1-(3,4-dichlorophenyl)-3-azabicyclo[3.1.0]hexane has been proposed as a candidate broad spectrum antidepressant, to possibly yield a more rapid onset and/or higher efficacy antidepressant effect than existing agents, including agents that inhibit single or dual reuptake of serotonin and/or norepinephrine (Skolnick, P., J. Clin. Psychiat., 63 [suppl. 2]:19-23, 2002; Skolnick, P., et al., Life Sci., 73: 3175-3179, 2003).

Available methods for synthesizing aryl substituted 3-azabicyclo[3.1.0]hexanes are limited. Bicifadine hydrochloride has been previously produced as described in U.S. Pat. No. 4,131,611, U.S. Pat. No. 4,196,120, U.S. Pat. No. 4,231,935, and in Epstein et al., J. Med. Chem. 24:481, 1981. An exemplary prior synthetic method for producing bicifadine hydrochloride is outlined in Scheme A, below.

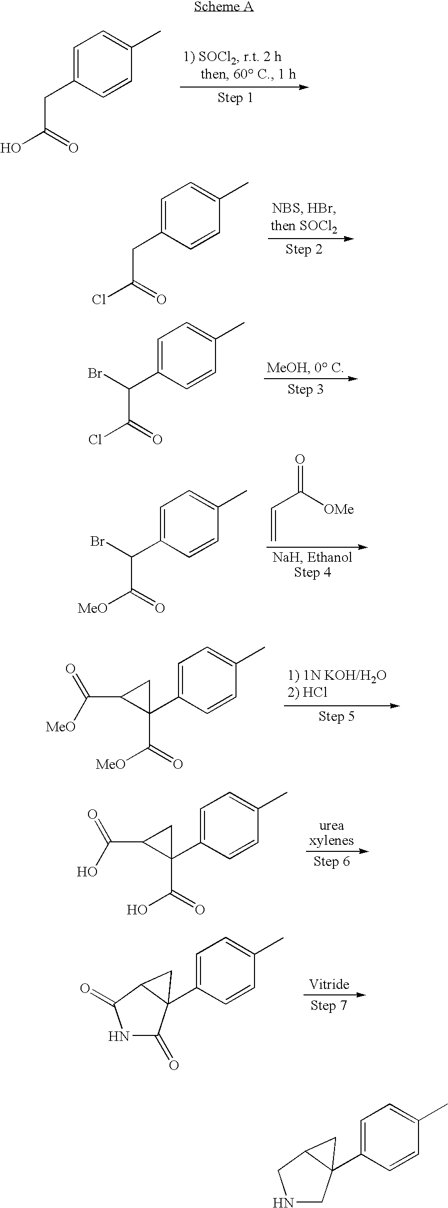

This synthetic scheme starts with preparation of the 2-bromo-2-(p-tolyl)-acetate in 3 steps. The dimethyl-1-(4-methylphenyl)-1,3-cyclopropanedicarboxylate is prepared from the bromoester by reaction with methyl acrylate. The diester is converted into the diacid, which is condensed with urea to produce 1-(p-tolyl)-1,2-cyclopropanedicarboximde. Then, the 1-(p-tolyl)-1-cyclopropanedicarboximde is reduced to an amine by Vitride and converted to the hydrochloride salt to yield the bicifadine hydrochloride.

U.S. Pat. No. 4,118,417 discloses a process for resolving a (+)-1-(p-methylphenyl)-1,2-cyclopropanedicarboxylic acid with S-(−)-1-(1-naphthyl)ethylamine, and its conversion to (+)-bicifadine, as illustrated below in synthetic Scheme B. The (−)-bicifadine is also reported to be producible from the corresponding (−)-1-(p-methylphenyl)-1,2-cyclopropanedicarboxylic acid.

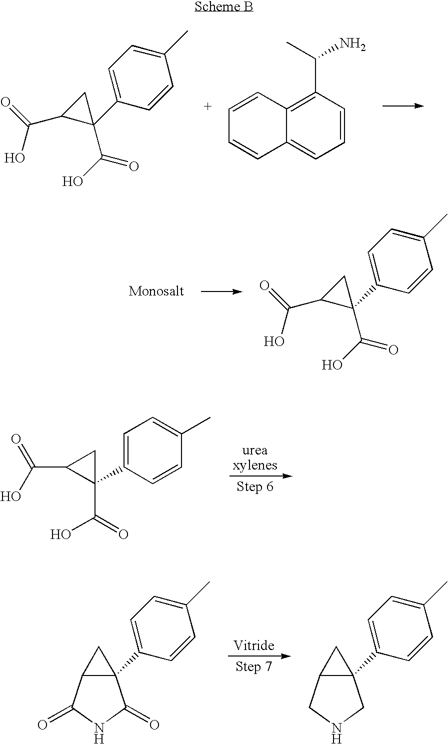

The foregoing synthetic methods provide limited tools for producing new 1-aryl-3-azabicyclo[3.1.0]hexanes, underscoring a need for additional methods and compositions to produce bicifadine and other substituted 1-aryl-3-azabicyclo[3.1.0]hexanes.

A related need exists to identify and develop new 1-aryl-3-azabicyclo[3.1.0]hexanes, along with new methods and compositions for producing, formulating and using these compounds as therapeutic tools.

It is therefore an object of the present invention to produce and select novel 1-aryl-3-azabicyclo[3.1.0]hexanes as candidate therapeutic agents.

It is a further object of the invention to provide new synthetic methods and compositions useful for producing 1-aryl-3-azabicyclo[3.1.0]hexanes and related compounds.

It is a further object of the invention to provide novel compositions and methods to treat central nervous system (CNS) disorders in mammals. Targeted CNS disorders in this context include a variety of serious neurologic and psychiatric conditions that are amenable to treatment or other beneficial intervention using an active agent capable of inhibiting biogenic amine transport, for example by inhibiting reuptake of norepinephrine and/or serotonin and/or dopamine.

It is a related object of the invention to provide novel 1-aryl-3-azabicyclo[3.1.0]hexane compositions and methods useful to treat or manage CNS disorders by modulating transport of one or more biogenic amines, for example to simultaneously inhibit or block reuptake of norepinephrine and/or serotonin and/or dopamine.

### SUMMARY OF EXEMPLARY EMBODIMENTS OF THE INVENTION

The invention achieves these objects and satisfies additional objects and advantages by providing novel 1-aryl-3-azabicyclo[3.1.0]hexanes that possess unexpected activities for modulating biogenic amine transport.

In certain embodiments of the invention, novel 1-aryl-3-azabicyclo[3.1.0]hexanes are provided that have at least one substituent on the aryl ring.

In other embodiments of the invention, novel 3-substituted 1-aryl-3-azabicyclo[3.1.0]hexanes are provided that have a substitution on the nitrogen at the ‘3’ position.

In additional embodiments of the invention, bi-substituted 1-aryl-3-azabicyclo[3.1.0]hexanes are provided which have at least one substitution on the aryl ring, as well as a substitution on the nitrogen at the ‘3’ position.

In exemplary embodiments, novel 1-aryl-3-azabicyclo[3.1.0]hexanes of the invention are characterized in part by formula II, below: 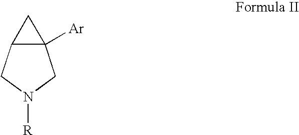
wherein Ar is a phenyl or other aromatic group having at least one substitution on the aryl ring, and wherein R is selected from, for example, hydrogen, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, di(C₁₋₃)alkylamino(C₁₋₆)alkyl, cyano(C₁₋₆)alkyl, methyl, ethyl, trifluoromethyl, trifluoroethyl and 2-methoxyethyl.

The invention also provides novel methods of making aryl- and aza-substituted 1-aryl-3-azabicyclo[3.1.0]hexanes, including synthetic methods that form novel intermediate compounds of the invention for producing aryl- and aza-substituted 1-aryl-3-azabicyclo[3.1.0]hexanes. In related embodiments, the invention provides novel processes for preparing one or more aryl- and/or aza-substituted 1-aryl-3-azabicyclo[3.1.0]hexanes, to yield novel compounds useful in biologically active and/or therapeutic compositions.

Useful 1-aryl-3-azabicyclo[3.1.0]hexanes of the invention include the substituted and bi-substituted 1-aryl-3-azabicyclo[3.1.0]hexane compounds described herein, as well as their active, pharmaceutically acceptable salts, polymorphs, solvates, hydrates and/or prodrugs, or combinations thereof.

In yet additional embodiments, the invention provides pharmaceutical compositions and methods for treating disorders of the central nervous system (CNS) including a wide array of serious neurological or psychiatric conditions, in mammals that are amenable to treatment using agents that inhibit or otherwise modulate biogenic amine transport.

The forgoing objects and additional objects, features, aspects and advantages of the present invention are further exemplified and described in the following detailed description.

### DETAILED DESCRIPTION OF EXEMPLARY EMBODIMENTS OF THE INVENTION

The instant invention provides novel, aryl-substituted and/or aza-substituted 1-aryl-3-azabicyclo[3.1.0]hexanes, as well as compositions and processes for producing these compounds. In exemplary embodiments, the invention provides compounds characterized in part by formula II, below: 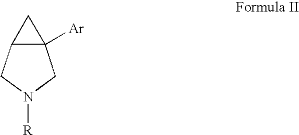
wherein Ar is a phenyl or other aryl group, optionally having at least one substitution on the aryl ring, and wherein R is H or an optional substituent selected from, for example, hydrogen, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, di(C₁₋₃)alkylamino(C₁₋₆)alkyl and cyano(C₁₋₆)alkyl, more preferably, methyl, ethyl, trifluoromethyl, trifluoroethyl and 2-methoxyethyl.

An illustrative assemblage of aryl substituted 1-aryl-3-azabicyclo[3.1.0]hexanes within this aspect of the invention is provided in Table 1, below. In each of these exemplary compounds, there is a methyl on the nitrogen at the ‘3’ position, however it is futher contemplated that the exemplified aryl subtitutions can be combined with other aza substitutions as described below to yield additional “bisubstituted” compounds as candidates for treating CNS disorders as described herein.

TABLE 1
Exemplary Aryl-Substituted 1-aryl-3-azabicyclo[3.1.0] hexanes

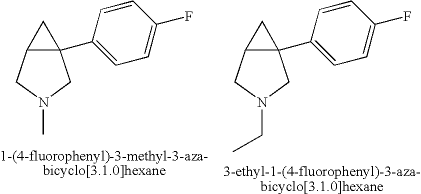

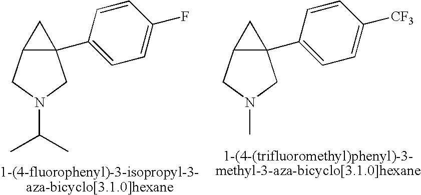

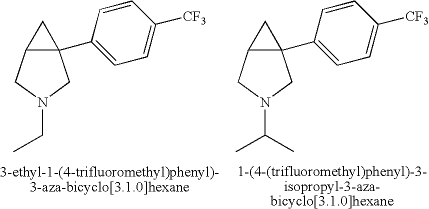

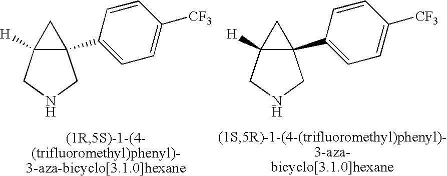

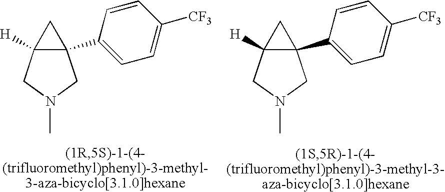

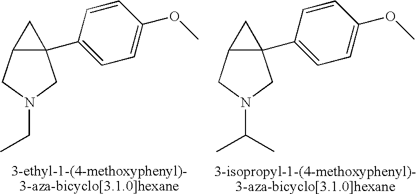

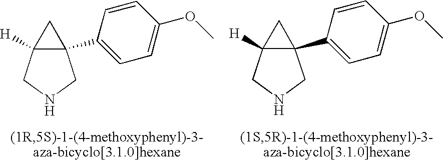

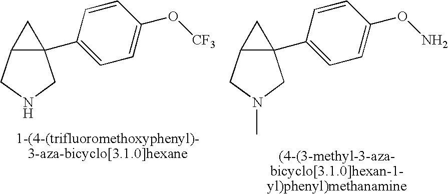

The aryl-substituted and aza-substituted 1-aryl-3-azabicyclo[3.1.0]hexanes of the invention are provided in any of a variety of forms, including pharmaceutically acceptable, active salts, solvates, hydrates, polymorphs, and/or prodrugs of the compounds disclosed herein, or any combination thereof.

In more detailed embodiments, the invention provides “bi-substituted” 1-aryl-3-azabicyclo[3.1.0]hexanes that have at least one substitution on the aryl ring and are also aza-subsituted, i.e., as characterized in part by formula III, below:

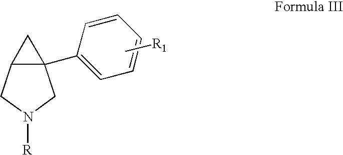

wherein R is selected from, for example, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, di(C₁₋₃)alkylamino(C₁₋₆)alkyl and cyano(C₁₋₆)alkyl, more preferably, methyl, ethyl, trifluoromethyl, trifluoroethyl and

2-methoxyethyl; and

wherein R₁ is selected from, for example, halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino, methyl, ethyl, fluoro, chloro, trifluoromethyl, cyano, nitro, phenyl and trifluoromethoxy.

In certain embodiments, these bi-substituted (aryl- and aza-substituted) compounds of the invention are characterized in part by the following formula IV, which describes in an exemplary manner a methyl substitution on the aryl ring at the same position as found in bicifadine:

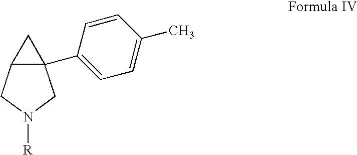

wherein R is selected from, for example, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, di(C₁₋₃)alkylamino(C₁₋₆)alkyl and cyano(C₁₋₆)alkyl, more preferably, methyl, ethyl, trifluoromethyl, trifluoroethyl and 2-methoxyethyl.

An illustrative assemblage of bi-substituted 1-aryl-3-azabicyclo[3.1.0]hexanes within this aspect of the invention is provided in Table 2. In each of these exemplary compounds, the hydrogen associated with the nitrogen at the ‘3’ position has been replaced with a different substituent as shown below.

TABLE 2
Exemplary Aza-Substituted 1-aryl-3-azabicyclo[3.1.0] hexanes

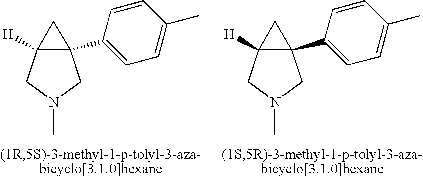

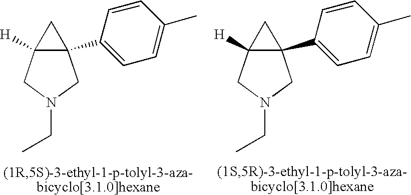

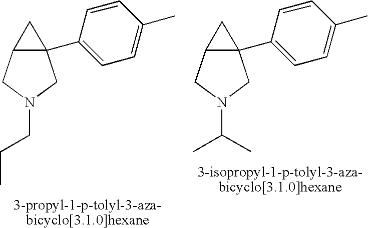

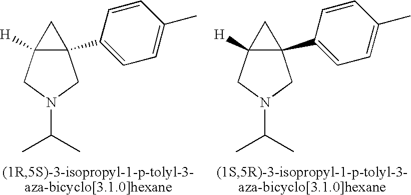

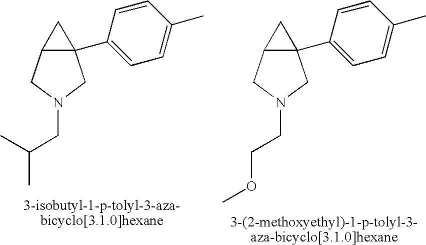

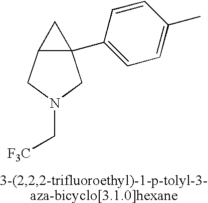

Also provided are novel methods and compositions for producing these and other 1-aryl-3-azabicyclo[3.1.0]hexanes. In particular, the present invention provides methods for making 1-aryl-3-azabicyclo[3.1.0]hexanes having the following formula III

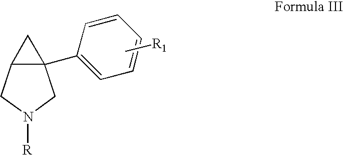

wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino, methyl, ethyl, fluoro, chloro, trifluoromethyl, cyano, nitro, phenyl or trifluoromethoxy

and R is hydrogen, and enantiomers, diastereomers and pharmaceutically acceptable salts thereof, comprising the steps of:
(a) reacting an aryl acetonitrile with epichlorohydrin to produce 2-(hydroxymethyl)-1-arylcyclopropanecarbonitrile;
(b) reducing the 2-(hydroxymethyl)-1-arylcyclopropanecarbonitrile to produce (2-(aminomethyl)-2-arylcyclopropyl)methanol;
(c) causing cyclization of the (2-(aminomethyl)-2-arylcyclopropyl)methanol to produce the 1-aryl-3-azabicyclo[3.1.0]hexane; and
(d) optionally converting the 1-aryl-3-azabicyclo[3.1.0]hexane to a pharmaceutically acceptable salt.

The present invention also provides methods for making a (1R, 5S) enantiomer of a 1-aryl-3-azabicyclo[3.1.0]hexane of the following formula III 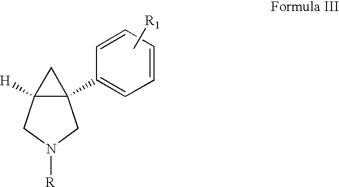
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, amino, C₁₋₃ alkylamino, di(C₁₋₃)alkylamino, methyl, ethyl, fluoro, chloro, trifluoromethyl, nitro, phenyl or trifluoromethoxy and R is hydrogen, and pharmaceutically acceptable salts thereof, comprising the steps of:
(a) reacting a compound of the following formula (i), 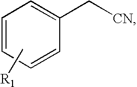
with (S)-(+)-epichlorohydrin to produce a compound of the following formula (ii), 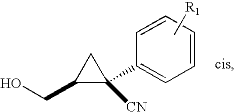
formula (iii), 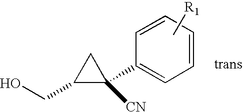
and formula (iv), 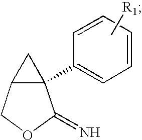
(b) reducing the compounds produced in step (a) to produce a compound of the following formula (v), 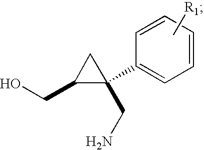
(c) causing cyclization of the compound of formula (v) to produce the (1R, 5S) enantiomer of the compound of Formula III; and
(d) optionally converting the (1R, 5S) enantiomer of the compound of Formula III to a pharmaceutically acceptable salt.

The present invention further provides methods for making a (1S, 5R) enantiomer of a 1-aryl-3-azabicyclo[3.1.0]hexane of the following formula III 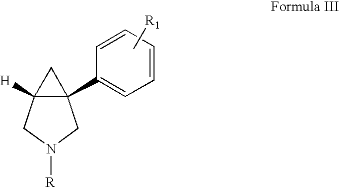
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, amino, C₁₋₃ alkylamino, di(C₁₋₃)alkylamino, methyl, ethyl, fluoro, chloro, trifluoromethyl, nitro, phenyl or trifluoromethoxy and R is hydrogen, and and pharmaceutically acceptable salts thereof, comprising the steps of:
(a) reacting a compound of the following formula (i), 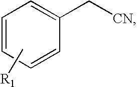
with (R)-(−)-epichlorohydrin to produce a compound of the following formula (vi), 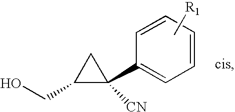
formula (vii), 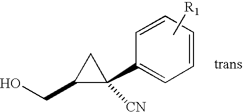
and formula (viii), 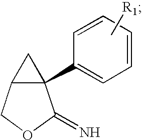
(b) reducing the compounds produced in step (a) to produce a compound of the following formula (ix), 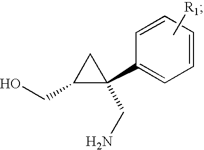
(c) causing cyclization of the compound of formula (ix) to produce the (1S, 5R) enantiomer of the compound of Formula III; and
(d) optionally converting the (1S, 5R) enantiomer of the compound of Formula III to a pharmaceutically acceptable salt.

The present invention additionally provides methods for making (1R,5S)-(+)-1-p-tolyl-3-azabicyclo[3.1.0]hexane and pharmaceutically acceptable salts thereof, comprising the steps of:
(a) reacting 1-p-tolylacetonitrile with S-(+)-epichlorohydrin to produce (1R,2S)-2-(hydroxymethyl)-1-p-tolylcyclopropanecarbonitrile;
(b) reducing the (1R, 2S)-2-(hydroxymethyl)-1-p-tolylcyclopropanecarbonitrile to produce ((1S, 2R)-2-(aminomethyl)-2-p-tolylcyclopropyl)methanol;
(c) causing cyclization of the ((1S, 2R)-2-(aminomethyl)-2-p-tolylcyclopropyl)methanol to produce (1R, 5S)-(+)-1-p-tolyl-3-azabicyclo[3.1.0]hexane; and
(d) optionally converting the (1R, 5S)-(+)-1-p-tolyl-3-azabicyclo[3.1.0]hexane into a pharmaceutically acceptable salt.

The present invention also provides methods for making (1S, 5R)-(−)-1-p-tolyl-3-azabicyclo[3.1.0]hexane and pharmaceutically acceptable salts thereof, comprising the steps of:
(a) reacting 1-p-tolylacetonitrile with R-(−)-epichlorohydrin to produce (1S, 2R)-2-hydroxymethyl-1-p-tolyl-cyclopropancarbonitrile;
(b) reducing the (1S, 2R)-2-hydroxymethyl-1-p-tolyl-cyclopropancarbonitrile to produce ((1R,2S)-2-(aminomethyl)-2-p-tolylcyclopropyl)methanol;
(c) causing cyclization of the ((1R,2S)-2-(aminomethyl)-2-p-tolylcyclopropyl)methanol to produce (1S, 5R)-(−)-1-p-Tolyl-3-azabicyclo[3.1.0]hexane; and
(d) optionally converting the (1S, 5R)-(−)-1-p-tolyl-3-azabicyclo[3.1.0]hexane into a pharmaceutically acceptable salt.

The present invention further provides methods for making a 1-aryl-3-azabicyclo[3.1.0]hexane of the following formula II, 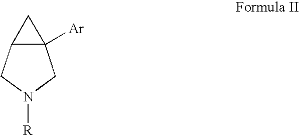
wherein R is hydrogen, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, di(C₁₋₃)alkylamino(C₁₋₆)alkyl, cyano(C₁₋₆)alkyl, methyl, ethyl, trifluoromethyl, trifluoroethyl or 2-methoxyethyl or C₁₋₆ alkyl and Ar is a monosubstituted phenyl group of the following formula (x), 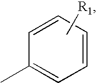
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, di(C₁₋₃)alkylamino, methyl, ethyl, fluoro, chloro, trifluoromethyl, nitro, phenyl or trifluoromethoxy, and enantiomers, diastereomers and pharmaceutically acceptable salts thereof, comprising the steps of:
(a) coupling a compound of the following formula (xi), 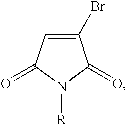
wherein R is a nitrogen protecting group, with a compound of the following formula (xii), ArB(OH)₂, wherein Ar is as defined above, to produce a compound of the following formula (xiii), 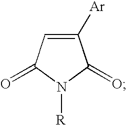
(b) causing cyclopropanation of the compound of formula (xiii) to produce a compound of the following formula (xiv), 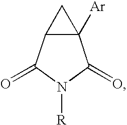
wherein Ar is as defined above and R is a nitrogen protecting group;
(c) reducing the compound of formula (xiv) to produce a compound of the following formula (xv), 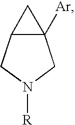
wherein Ar is as defined above and R is a nitrogen protecting group;
(d) deprotecting the compound of formula (xv) to produce the 1-aryl-3-azabicyclo[3.1.0]hexane; and
(e) optionally converting the 1-aryl-3-azabicyclo[3.1.0]hexane to a pharmaceutically acceptable salt.

The present invention additionally provides methods for resolving 1-aryl-3-aza-bicyclo[3.1.0]hexanes of the following formula III 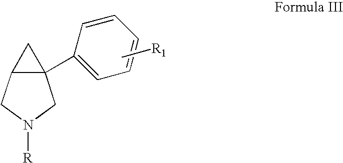
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino, methyl, ethyl, fluoro, chloro, trifluoromethyl, cyano, nitro, phenyl or trifluoromethoxy and R is hydrogen, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, di(C₁₋₃)alkylamino(C₁₋₆)alkyl, cyano(C₁₋₆)alkyl, methyl, ethyl, trifluoromethyl, trifluoroethyl or 2-methoxyethyl to a (+)- or (−)-enantiomer of the compound of Formula I, and pharmaceutically acceptable salts thereof, comprising the following steps:
(a) reacting the compound of Formula III with either a (+) or (−) enantiomer of tartaric acid to produce a tartrate salt of the compound of Formula III;
(b) reacting the tartrate salt of the compound of Formula III produced in step (a) with a base to produce a free base of the (+) or (−) enantiomer of the compound of Formula III; and
(c) optionally converting the free base of the (+) or (−) enantiomer of the compound of Formula III to a pharmaceutically acceptable salt.
   The present invention also provides methods for making a 1-aryl-3-azabicyclo[3.1.0]hexane of the following Formula III 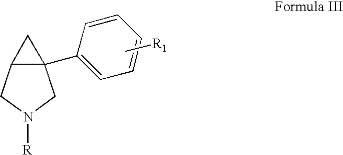
   wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, di(C₁₋₃)alkylamino, methyl, ethyl, fluoro, chloro, trifluoromethyl, phenyl or trifluoromethoxy and R is hydrogen, and enantiomers, diastereomers and pharmaceutically acceptable salts thereof, comprising the steps of:
(a) reacting a compound of the following formula (xi), 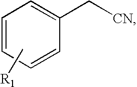
wherein R₁ is as defined above, with
epichlorohydrin to produce a compound of the following formula (xii), 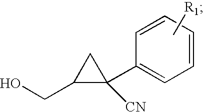
(b) reducing the compound of the formula (xii) to produce a compound of the following formula (xiii), 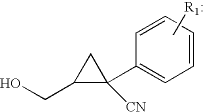
(c) reacting the compound of the formula (xiii) with (Boc)₂O to produce a compound of the following formula (xiv), 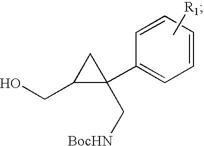
(d) causing cyclization of the compound of the formula (xiv) to produce a compound of the following formula (xv), 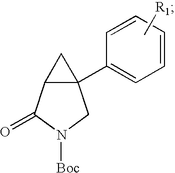
(e) deprotecting the compound of the formula (xv) to produce the compound of the following formula (xvi), 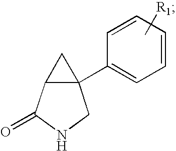
(f) reducing the compound of the formula (xvi) to produce the compound of Formula III; and
(g) optionally converting the compound of Formula III to a pharmaceutically acceptable salt.

In practicing the methods of the present for methods for making 1-aryl-3-azabicyclo[3.1.0]hexanes, various reagents may be utilized for the different reaction steps. In general, suitable reagents for the various reaction steps may be selected by one of ordinary skill in the art based on the present disclosure.

Suitable reducing agents and methodologies include, for example, lithium aluminum hydride (LAH), sodium aluminum hydride (SAH), NaBH₄ with ZnCl₂ and catalytic hydrogenation.

Suitable nitrogen protecting groups include, for example, benzyl, allyl, tert-butyl and 3,4-dimethoxy-benzyl groups. In general, nitrogen protecting groups are well known to those skilled in the art, see for example, “Nitrogen Protecting Groups in Organic Synthesis”, John Wiley and sons, New York, N.Y., 1981, Chapter 7; “Nitrogen Protecting Groups in Organic Chemistry”, Plenum Press, New York, N.Y., 1973, Chapter 2; See also, T. W. Green and P. G. M. Wuts in “Protective Groups in Organic Chemistry, 3rd edition” John Wiley & Sons, Inc. New York, N.Y., 1999.

When the nitrogen protecting group is no longer needed, it may be removed by methods well known in the art. For example, benzyl or 3,4-dimethoxy-benzyl groups may be removed by catalytic hydrogenation. In general, methods of removing nitrogen protecting groups are well known to those skilled in the art, see for example, “Nitrogen Protecting Groups in Organic Synthesis”, John Wiley and sons, New York, N.Y., 1981, Chapter 7; “Nitrogen Protecting Groups in Organic Chemistry”, Plenum Press, New York, N.Y., 1973, Chapter 2; See also, T. W. Green and P. G. M. Wuts in “Protective Groups in Organic Chemistry, 3rd edition” John Wiley & Sons, Inc. New York, N.Y., 1999.

Suitable reagents for causing cyclization include, for example, SOCl₂, POCl₃, oxalyl chloride, phosphorous tribromide, triphenylphosphorous dibromide and oxalyl bromide.

For the purposes of further describing the invention, including the novel compounds and synthetic methods disclosed herein, the following terms and definitions are provided by way of example.

The term “halogen” as used herein refers to bromine, chlorine, fluorine or iodine. In one embodiment, the halogen is chlorine. In another embodiment, the halogen is bromine.

The term “hydroxy” as used herein refers to —OH or —O—.

The term “alkyl” as used herein refers to straight- or branched-chain aliphatic groups containing 1-20 carbon atoms, preferably 1-7 carbon atoms and most preferably 1-4 carbon atoms. This definition applies as well to the alkyl portion of alkoxy, alkanoyl and aralkyl groups. In one embodiment, the alkyl is a methyl group.

The term “alkoxy” includes substituted and unsubstituted alkyl, alkenyl, and alkynyl groups covalently linked to an oxygen atom. In one embodiment, the alkoxy group contains 1 to 4 carbon atoms. Embodiments of alkoxy groups include, but are not limited to, methoxy, ethoxy, isopropyloxy, propoxy, butoxy, and pentoxy groups. Embodiments of substituted alkoxy groups include halogenated alkoxy groups. In a further embodiment, the alkoxy groups can be substituted with groups such as alkenyl, alkynyl, halogen, hydroxyl, alkylcarbonyloxy, phenylcarbonyloxy, alkoxycarbonyloxy, phenyloxycarbonyloxy, carboxylate, alkylcarbonyl, phenylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, phenylamino, diphenylamino, and alkylphenylamino), acylamino (including alkylcarbonylamino, phenylcarbonylamino, carbamoyl and ureido), amidino, imino, sulfhydryl, alkylthio, phenylthio, thiocarboxylate, sulfates, alkylsulfinyl, sulfonato, sulfamoyl, sulfonamido, nitro, trifluoromethyl, cyano, azido, heterocyclyl, alkylphenyl, or aromatic or heteroaromatic moieties. Exemplary halogen substituted alkoxy groups include, but are not limited to, fluoromethoxy, difluoromethoxy, trifluoromethoxy, chloromethoxy, dichloromethoxy, and trichloromethoxy.

The term “nitro”, as used herein alone or in combination refers to a —NO₂ group.

The term “amino” as used herein refers to the group —NRR′, where R and R′ may independently be hydrogen, alkyl, phenyl, alkoxy, or heterophenyl. The term “aminoalkyl” as used herein represents a more detailed selection as compared to “amino” and refers to the group —NRR′, where R and R′ may independently be hydrogen or (C₁-C₄)alkyl.

The term “trifluoromethyl” as used herein refers to —CF₃.

The term “trifluoromethoxy” as used herein refers to —OCF₃.

The term “cycloalkyl” as used herein refers to a saturated cyclic hydrocarbon ring system containing from 3 to 7 carbon atoms that may be optionally substituted. Exemplary embodiments include, but are not limited to, cyclopropyl, cyclobutyl, cyclopentyl and cyclohexyl. In certain embodiments, the cycloalkyl group is cyclopropyl. In another embodiment, the (cycloalkyl)alkyl groups contain from 3 to 7 carbon atoms in the cyclic portion and 1 to 4 carbon atoms in the alkyl portion. In certain embodiments, the (cycloalkyl)alkyl group is cyclopropylmethyl. The alkyl groups are optionally substituted with from one to three substituents selected from the group consisting of halogen, hydroxy and amino.

The terms “alkanoyl” and “alkanoyloxy” as used herein refer, respectively, to C(O)-alkyl groups and —O—C(O)-alkyl groups, each optionally containing 2-5 carbon atoms. Specific embodiments of alkanoyl and alkanoyloxy groups are acetyl and acetoxy, respectively.

The term “aroyl,” as used alone or in combination herein, refers to a phenyl radical derived from an aromatic carboxylic acid, such as optionally substituted benzoic or naphthoic acids.

The term “aralkyl” as used herein refers to a phenyl group bonded to an alkyl group, preferably one containing 1-4 carbon atoms. A preferred aralkyl group is benzyl.

The term “nitrile” or “cyano” as used herein refers to the group —CN.

The term “pyrrolidine-1-yl” as used herein refers to the structure:

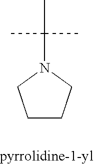

The term “morpholino” as used herein refers to the structure:

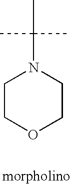

The term “dialkylamino” refers to an amino group having two attached alkyl groups that can be the same or different.

The term “alkenyl” refers to a straight or branched alkenyl group of 2 to 10 carbon atoms having 1 to 3 double bonds. Preferred embodiments include ethenyl, 1-propenyl, 2-propenyl, 1-methylethenyl, 1-butenyl, 2-butenyl, 3-butenyl, 2-methyl-2-propenyl, 1-pentenyl, 2-pentenyl, 4-pentenyl, 3-methyl-2-butenyl, 1-hexenyl, 2-hexenyl, 1-heptenyl, 2-heptenyl, 1-octenyl, 2-octenyl, 1,3-octadienyl, 2-nonenyl, 1,3-nonadienyl, 2-decenyl, etc.

The term “alkynyl” as used herein refers to a straight or branched alkynyl group of 2 to 10 carbon atoms having 1 to 3 triple bonds. Exemplary alkynyls include, but are not limited to, ethynyl, 1-propynyl, 2-propynyl, 1-butynyl, 2-butynyl, 3-butynyl, 1-pentynyl, 2-pentynyl, 4-pentynyl, 1-octynyl, 6-methyl-1-heptynyl, and 2-decynyl.

The term “hydroxyalkyl” alone or in combination, refers to an alkyl group as previously defined, wherein one or several hydrogen atoms, preferably one hydrogen atom has been replaced by a hydroxyl group. Examples include hydroxymethyl, hydroxyethyl and 2-hydroxyethyl.

The term “aminoalkyl” as used herein refers to the group —NRR′, where R and R′ may independently be hydrogen or (C₁-C₄)alkyl.

The term “alkylaminoalkyl” refers to an alkylamino group linked via an alkyl group (i.e., a group having the general structure -alkyl-NH-alkyl or -alkyl-N(alkyl)(alkyl)). Such groups include, but are not limited to, mono- and di-(C₁-C₈ alkyl)aminoC₁-C₈ alkyl, in which each alkyl may be the same or different.

The term “dialkylaminoalkyl” refers to alkylamino groups attached to an alkyl group. Examples include, but are not limited to, N,N-dimethylaminomethyl, N,N-dimethylaminoethyl, N,N-dimethylaminopropyl, and the like. The term dialkylaminoalkyl also includes groups where the bridging alkyl moiety is optionally substituted.

The term “haloalkyl” refers to an alkyl group substituted with one or more halo groups, for example chloromethyl, 2-bromoethyl, 3-iodopropyl, trifluoromethyl, perfluoropropyl, 8-chlorononyl and the like.

The term “carboxyalkyl” as used herein refers to the substituent —R′—COOH wherein R′ is alkylene; and carbalkoxyalkyl refers to —R′—COOR wherein R′ and R are alkylene and alkyl respectively. In certain embodiments, alkyl refers to a saturated straight- or branched-chain hydrocarbyl radical of 1-6 carbon atoms such as methyl, ethyl, n-propyl, isopropyl, n-butyl, t-butyl, n-pentyl, 2-methylpentyl, n-hexyl, and so forth. Alkylene is the same as alkyl except that the group is divalent.

The term “alkoxyalkyl” refers to an alkylene group substituted with an alkoxy group. For example, methoxyethyl [CH₃OCH₂CH₂—] and ethoxymethyl (CH₃CH₂OCH₂—] are both C₃ alkoxyalkyl groups.

The term “carboxy”, as used herein, represents a group of the formula —COOH.

The term “alkanoylamino” refers to alkyl, alkenyl or alkynyl groups containing the group —C(O)— followed by —N(H)—, for example acetylamino, propanoylamino and butanoylamino and the like.

The term “carbonylamino” refers to the group —NR—CO—CH₂—R′, where R and R′ may be independently selected from hydrogen or (C₁-C₄)alkyl.

The term “carbamoyl” as used herein refers to —O—C(O)NH₂.

The term “carbamyl” as used herein refers to a functional group in which a nitrogen atom is directly bonded to a carbonyl, i.e., as in —NRC(═O)R′ or —C(═O)NRR′, wherein R and R′ can be hydrogen, alkyl, substituted alkyl, alkenyl, substituted alkenyl, alkoxy, cycloalkyl, phenyl, heterocyclo, or heterophenyl.

The term “alkylsulfonylamino” refers to refers to the group —NHS(O)₂Rₐ wherein Rₐ is an alkyl as defined above.

As noted above, the compounds of the present invention can be can be prepared as both acid addition salts formed from an acid and the basic nitrogen group of 1-aryl-3-azabicyclo[3.1.0]hexanes and base salts. As further noted above, the methods of the present invention can be used to prepare compounds as both acid addition salts formed from an acid and the basic nitrogen group of 1-aryl-3-azabicyclo[3.1.0]hexanes and base salts. Suitable acid addition salts include, for example, hydrochloride, hydrobromide, hydroiodide, sulphate, hydrogen sulphate, nitrate, phosphate, and hydrogen phosphate salts. Other examples of pharmaceutically acceptable acid addition salts include inorganic and organic acid addition salts. Additional pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate, tartrate, gluconate and the like. Suitable base salts are formed from bases, which form non-toxic salts and include, for example, aluminum, calcium, lithium, magnesium, potassium, sodium, zinc and diethanolamine salts. The hydrochloride salt formed with hydrochloric acid is an exemplary useful salt.

The compositions and methods of the instant invention comprising a 1-aryl-3-azabicyclo[3.1.0]hexane are effective for treating or preventing a variety of central nervous system (CNS) disorders in mammals. In certain embodiments, pharmaceutical compositions and methods are provided for treating a CNS disorder in a mammalian subject. Mammalian subjects amenable for treatment using these compositions and methods include, but are not limited to, human and other mammalian subjects suffering from a CNS disorder that responds positively to intervention by inhibition of biogenic amine transport. In related embodiments, therapeutic compositions and methods are provided which employ an effective amount of one or more 1-aryl-3-azabicyclo[3.1.0]hexane(s) described herein to treat or prevent a selected CNS disorder in a subject, wherein administration of the composition to the subject effectively inhibits the function of one or more, and in certain embodiments all three, norepinephrine, serotonin, and/or dopamine transport proteins in the subject, thereby preventing, or reducing the occurrence or severity of symptoms of, the targeted CNS disorder.

In related embodiments, a biogenic amine transport inhibitory-effective amount of an aryl substituted 1-aryl-3-azabicyclo[3.1.0]hexane of the invention is administered to treat or prevent a CNS disorder, including neurological or psychiatric conditions, in a mammalian subject responsive to inhibition of biogenic amine transport. In more detailed aspects, administration of an active compound of the invention provides a therapeutic or prophylactic benefit by inhibiting or blocking reuptake of one or more, including any combination of two, or all three, biogenic amines selected from norepinephrine, serotonin, and dopamine.

Within more detailed treatment methods of the invention, administration of the active 1-aryl-3-azabicyclo[3.1.0]hexane(s) mediates a therapeutic effect via the active compound inhibiting reuptake of norepinephrine, serotonin, and/or dopamine. Biogenic amine reuptake inhibition in this context can optionally be determined and selected by using one or more 1-aryl-3-azabicyclo[3.1.0]hexane(s) of the invention to achieve variable selectivity and potency of transporter inhibition, wherein one or any combination of norepinephrine, serotonin and/or dopamine transporters can be inhibited, at pre-determined levels or ratios among or between different transporters. In this context, the various 1-aryl-3-azabicyclo[3.1.0]hexanes of the invention exhibit a wide range of potencies as inhibitors of one, two, or all three of the norepinephrine, serotonin and dopamine transporters—rendering them useful in a broad array of therapeutic applications.

In exemplary embodiments, the compositions and methods of the invention can be administered to mammalian subjects to measurably alleviate or prevent one or more symptoms of a CNS disorder, such as any neurological or psychiatric condition, for example, pain. The methods and compositions of the invention are also useful to treat non-pain-related psychiatric or neurological disorders, for example anxiety, appetite disorders, and depression.

Administration of an effective amount of a 1-aryl-3-azabicyclo[3.1.0]hexane of the invention to a mammalian subject presenting with one or more symptoms of a CNS disorder or other neurological or psychiatric condition will detectably decrease, eliminate, or prevent the subject symptom(s). In exemplary embodiments, administration of a 1-aryl-3-azabicyclo[3.1.0]hexane composition to a suitable test subject will yield a reduction in one or more target symptom(s) associated with a selected CNS disorder, such as pain, by at least 10%, 20%, 30%, 50% or greater, up to a 75-90%, or 95% or greater, reduction in the one or more target symptom(s), compared to placebo-treated or other suitable control subjects. Comparable levels of efficacy are contemplated for the entire range of CNS disorders, including all contemplated neurological and psychiatric disorders, and related conditions and symptoms, for treatment or prevention using the compositions and methods of the invention.

The active compounds of the invention may be optionally formulated with a pharmaceutically acceptable carrier and/or various excipients, vehicles, stabilizers, buffers, preservatives, etc. An “effective amount,” “therapeutic amount,” “therapeutically effective amount,” or “effective dose” is an effective amount or dose of an active compound as described herein sufficient to elicit a desired pharmacological or therapeutic effect in a mammalian subject—typically resulting in a measurable reduction in an occurrence, frequency, or severity of one or more symptom(s) of a CNS disorder, including any combination of neurological and/or psychological symptoms, diseases, or conditions, associated with or caused by the targeted CNS disorder, in the subject. In certain embodiments, when a compound of the invention is administered to treat a CNS disorder, for example a pain disorder, an effective amount of the compound will be an amount sufficient in vivo to delay or eliminate onset of symptoms of the targeted condition or disorder. Therapeutic efficacy can alternatively be demonstrated by a decrease in the frequency or severity of symptoms associated with the treated condition or disorder, or by altering the nature, recurrence, or duration of symptoms associated with the treated condition or disorder. Therapeutically effective amounts, and dosage regimens, of the 1-aryl-3-azabicyclo[3.1.0]hexane compositions of the invention, including pharmaceutically effective salts, solvates, hydrates, polymorphs or prodrugs thereof, will be readily determinable by those of ordinary skill in the art, often based on routine clinical or patient-specific factors.

Suitable routes of administration for a 1-aryl-3-azabicyclo[3.1.0]hexane of the invention include, but are not limited to, oral, buccal, nasal, aerosol, topical, transdermal, mucosal, injectable, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection.

Suitable effective unit dosage amounts of a 1-aryl-3-azabicyclo[3.1.0]hexane of the invention for mammalian subjects may range from about 25 to 1800 mg, 50 to 1000 mg, 75 to 900 mg, 100 to 750 mg, or 150 to 500 mg. In certain embodiments, the effective dosage will be selected within narrower ranges of, for example, 10 to 25 mg, 30-50 mg, 75 to 10 mg, 100 to 250 mg, or 250 to 500 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In exemplary embodiments, dosages of 10 to 25 mg, 30-50 mg, 75 to 100 mg, 100 to 250 mg, or 250 to 500 mg, are administered one, two, three, or four times per day. In more detailed embodiments, dosages of 50-75 mg, 100-200 mg, 250400 mg, or 400-600 mg are administered once or twice daily. In alternate embodiments, dosages are calculated based on body weight, and may be administered, for example, in amounts from about 0.5 mg/kg to about 20 mg/kg per day, 1 mg/kg to about 15 mg/kg per day, 1 mg/kg to about 10 mg/kg per day, 2 mg/kg to about 20 mg/kg per day, 2 mg/kg to about 10 mg/kg per day or 3 mg/kg to about 15 mg/kg per day.

The amount, timing and mode of delivery of compositions of the invention comprising an effective amount of a 1-aryl-3-azabicyclo[3.1.0]hexane of the invention will be routinely adjusted on an individual basis, depending on such factors as weight, age, gender, and condition of the individual, the acuteness of the condition to be treated and/or related symptoms, whether the administration is prophylactic or therapeutic, and on the basis of other factors known to effect drug delivery, absorption, pharmacokinetics, including half-life, and efficacy. An effective dose or multi-dose treatment regimen for the compounds of the invention will ordinarily be selected to approximate a minimal dosing regimen that is necessary and sufficient to substantially prevent or alleviate one or more symptom(s) of a neurological or psychiatric condition in the subject, as described herein. Thus, following administration of a 1-aryl-3-azabicyclo[3.1.0]hexane of the invention according to the formulations and methods herein, test subjects will exhibit a 10%, 20%, 30%, 50% or greater reduction, up to a 75-90%, or 95% or greater, reduction, in one or more symptoms associated with a targeted CNS disorder or other neurological or psychiatric condition, compared to placebo-treated or other suitable control subjects.

Pharmaceutical dosage forms of the 1-aryl-3-azabicyclo[3.1.0]hexanes of the present invention may optionally include excipients recognized in the art of pharmaceutical compounding as being suitable for the preparation of dosage units as discussed above. Such excipients include, without limitation, binders, fillers, lubricants, emulsifiers, suspending agents, sweeteners, flavorings, preservatives, buffers, wetting agents, disintegrants, effervescent agents and other conventional excipients and additives.

The compositions of the invention for treating CNS disorders, including depression, anxiety, and/or pain, can thus include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives and agents will often be biologically inactive and can be administered to patients without causing unacceptable deleterious side effects or serious adverse interactions with the active agent.

If desired, the substituted 1-aryl-3-azabicyclo[3.1.0]hexanes of the invention can be administered in a controlled release form, for example by use of a slow release carrier such as a hydrophilic, slow release polymer. Exemplary controlled release agents in this context include, but are not limited to, hydroxypropyl methyl cellulose, having a viscosity in the range of about 100 cps to about 100,000 cps.

1-aryl-3-azabicyclo[3.1.0]hexane compositions of the invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose, dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets, which may be prepared by any conventional method of preparing pharmaceutical oral unit dosage form. Oral unit dosage forms, such as tablets, may contain one or more conventional additional formulation ingredients, including, but are not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds. By effervescent agent is meant a couple, typically an organic acid and a carbonate or bicarbonate. Such rapidly acting dosage forms would be useful, for example, in the prevention or treatment of acute attacks of panic disorder.

Additional 1-aryl-3-azabicyclo[3.1.0]hexane compositions of the invention can be prepared and administered in any of a variety of inhalation or nasal delivery forms known in the art. Devices capable of depositing aerosolized substituted 1-aryl-3-azabicyclo[3.1.0]hexane formulations in the sinus cavity or pulmonary alveoli of a patient include metered dose inhalers, nebulizers, dry powder generators, sprayers, and the like. Pulmonary delivery to the lungs for rapid transit across the alveolar epithelium into the blood stream may be particularly useful in treating impending episodes of seizures or panic disorder. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of a 1-aryl-3-azabicyclo[3.1.0]hexane, and any additional active or inactive ingredient(s).

Intranasal and pulmonary delivery permits the passage of active compounds of the invention into the blood stream directly after administering an effective amount of the compound to the nose or lung. In the case of intranasal delivery, this mode of administration can achieve direct, or enhanced, delivery of the active compound to the CNS. For intranasal and pulmonary administration, a liquid aerosol formulation will often contain an active compound of the invention combined with a dispersing agent and/or a physiologically acceptable diluent. Alternatively, dry powder aerosol formulations may contain a finely divided solid form of the subject compound and a dispersing agent allowing for the ready dispersal of the dry powder particles. With either liquid or dry powder aerosol formulations, the formulation must be aerosolized into small, liquid or solid particles in order to ensure that the aerosolized dose reaches the mucous membranes of the nasal passages or the lung. The term “aerosol particle” is used herein to describe a suitable liquid or solid particle of a sufficiently small particle diameter, e.g., in a range of from about 2-5 microns, for nasal or pulmonary distribution to targeted mucous or alveolar membranes. Other considerations include the construction of the delivery device, additional components in the formulation, and particle characteristics. These aspects of nasal or pulmonary administration of drugs are well known in the art, and manipulation of formulations, aerosolization means, and construction of delivery devices, is within the level of ordinary skill in the art.

Yet additional compositions and methods of the invention are provided for topical administration of 1-aryl-3-azabicyclo[3.1.0]hexanes for treating CNS disorders, including pain. Topical compositions may comprise a 1-aryl-3-azabicyclo[3.1.0]hexane and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise a 1-aryl-3-azabicyclo[3.1.0]hexane dissolved or dispersed in a portion of a water or other solvent or liquid to be incorporated in the topical composition or delivery device. Transdermal administration may be enhanced by the use of dermal penetration enhancers known to those skilled in the art.

Yet additional 1-aryl-3-azabicyclo[3.1.0]hexane formulations are provided for parenteral administration, including aqueous and non-aqueous sterile injection solutions which may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers.

1-aryl-3-azabicyclo[3.1.0]hexane formulations of the invention may also include polymers for extended release following parenteral administration. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

In more detailed embodiments, 1-aryl-3-azabicyclo[3.1.0]hexanes may be encapsulated for delivery in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions.

The invention also provides pharmaceutical packs or kits comprising one or more containers holding a 1-aryl-3-azabicyclo[3.1.0]hexane, or any composition comprising a 1-aryl-3-azabicyclo[3.1.0]hexane as described herein, including pharmaceutically acceptable salts and other forms of 1-aryl-3-azabicyclo[3.1.0]hexanes as described, in a pharmaceutically acceptable, stable form. Optionally packaged with these packs and kits can be a notice, e.g., in a form prescribed by a governmental agency regulating pharmaceuticals or biological products, reflecting approval by the agency of the manufacture, use and/or sale of the product contained in the pack or kit for human administration (optionally specifying one or more approved treatment indications as described herein).

Compounds and compositions of the present invention are also useful in a variety of in vitro applications, including a range of diagnostic uses. In exemplary in vitro assays, compounds and compositions of the invention can be used as CNS imaging agents. In other embodiments, the compounds of the invention can be used in a variety of conventional, clinical assays to determine whether it is desired to administer a compound of the present invention, or a particular dosage form or quantity of the compound, to a particular patient as a therapeutic agent. For example, assays employing cell cultures, tissue cultures, or animal model systems can be used to demonstrate safety and efficacy of the compounds and pharmaceutical formulations described herein. Additional uses of the compounds of the invention, e.g., in radiolabeled or other labeled form, can be used to study biochemical mechanisms, metabolic processes, pharmacokinetics, etc. of the subject compounds and/or their targets in a diverse array of in vitro, ex vivo, and in vivo assays. Each of the foregoing general applications of the subject compounds will be understood by those skilled in the art to have many corresponding embodiments and modified formats following conventional methods and procedures widely known in the art.

The following examples illustrate certain embodiments of the present invention, and are not to be construed as limiting the present disclosure.

### EXAMPLE I

### Synthetic Methods for Preparing Substituted 1-aryl-3-azabicyclo[3.1.01 hexanes

Although many of the novel 1-aryl-3-azabicyclo[3.1.0]hexanes of the invention may be prepared according to methods known to those skilled in the art, they may also be generated, for example, according to the exemplary reaction schemes set forth below. While these novel schemes employ various intermediates and starting materials, it is to be understood that the illustrated processes are also applicable to compounds having alternative structure, substituent patterns, or stereochemistry depicted in these schemes. Throughout Reaction Schemes 1 to 18 hereinbelow, R₁ is hydrogen, C₁₋₆ alkyl, halo(C₁₋₆)alkyl, C₃₋₉ cycloalkyl, C₁₋₅alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, di(C₁₋₃)alkylamino(C₁₋₆)alkyl, cyano(C₁₋₆)alkyl, methyl, ethyl, trifluoromethyl, trifluoroethyl or 2-methoxyethyl.

Reaction Scheme 1 below generally sets forth an exemplary process for preparing bicifadine and analogs from corresponding 2-bromo-2-arylacetate or 2-chloro-2-arylacetate. The bromo or chloro acetate react with acrylonitrile to provide the methyl 2-cyano-1-arylcyclopropanecarboxylate, which is then reduced into the amino alcohol by reducing agents such as lithium aluminum hydride (LAH) or sodium aluminum hydride (SAH) or NaBH₄ with ZnCl₂. Cyclization of the amino alcohol with SOCl₂ or POCl₃ will provide the 1-aryl-3-azabicyclo[3.1.0]hexane. The cyclization of substituted 4-aminobutan-1-ol by SOCl₂ or POCl₃ into the pyrrolidine ring system was reported by Armarego et al., J. Chem. Soc. [Section C: Organic] 19:3222-9, 1971, and in patent publication PL 120095 B2, CAN 99:158251 by Szalacke et al. Oxalyl chloride, phosphorous tribromide, triphenylphosphorous dibromide and oxalyl bromide may be used for the same purpose. The methyl 2-bromo-2-arylacetate or methyl 2-chloro-2-arylacetate may be synthesized from subsituted benzoylaldehyde or methyl-2-arylacetate as shown in Reaction Scheme 1A.

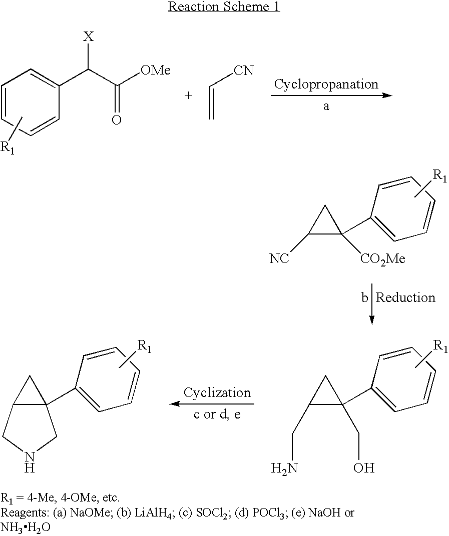

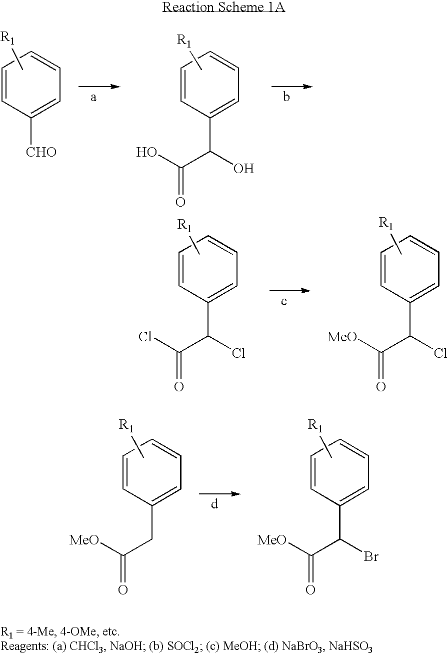

Reaction Scheme 2 below illustrates another exemplary process for transforming methyl 2-cyano-1-arylcyclopropanecarboxylate to a desired compound or intermediate of the invention. Hydrolysis of the cyano ester provides the potassium salt which can then be converted into the cyano acid. Reduction and cyclization of the 2-cyano-1-arylcyclopropanecarboxylic acid with LAH or LiAlH(OMe)₃ according to the procedure outlined in Tetrahedron 45:3683, 1989, will generate 1-aryl-3-azabicyclo[3.1.0]hexane. In addition, the cyano-1-arylcyclopropanecarboxylic acid can be hydrogenated and cyclized into an amide, which is then reduced into 1-aryl-3-azabicyclo[3.1.0]hexane.

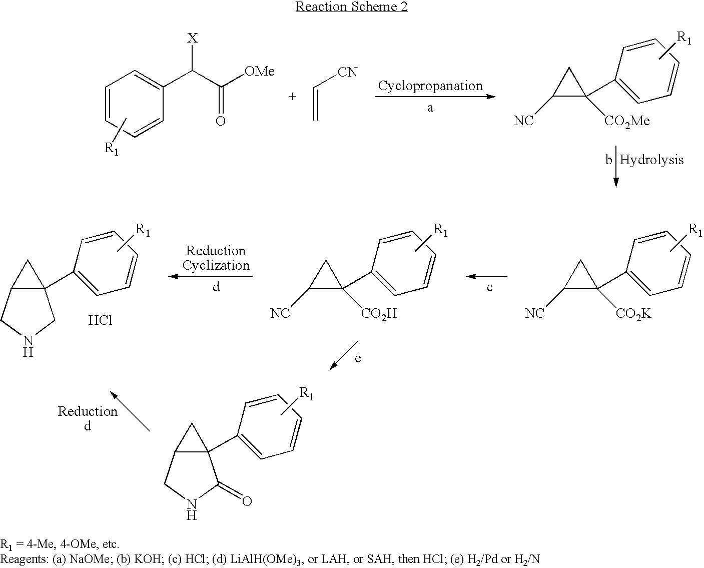

Reaction Scheme 3 below discloses an alternative exemplary process for converting the methyl 2-cyano-1-arylcyclopropanecarboxylate to a desired compound or intermediate of the invention. The methyl 2-cyano-1-arylcyclopropanecarboxylate is reduced and cyclized into 1-aryl-3-aza-bicyclo[3.1.0]hexan-2-one, which is then reduced to 1-aryl-3-azabicyclo[3.1.0]hexane (Marazzo et al., Arkivoc v:156-169, 2004).

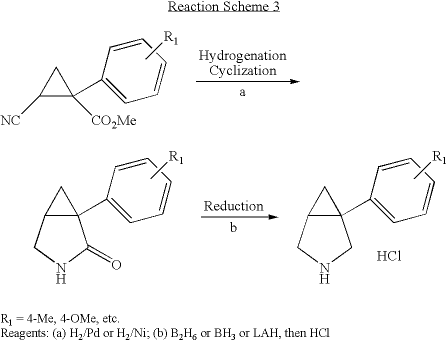

Reaction Scheme 4 below provides another exemplary process to prepare bicifadine and analogs. Reaction of 2-arylacetonitrile with (O)-epichlorohydrin gives approximately a 65% yield of 2-(hydroxymethyl)-1-arylcyclopropanecarbonitrile (85% cis) with the trans isomer as one of the by-products (Cabadio et al., Fr. Bollettino Chimico Farmaceutico 117:331-42, 1978; Mouzin et al., Synthesis 4:304-305, 1978). The methyl 2-cyano-1-arylcyclopropanecarboxylate can then be reduced into the amino alcohol by a reducing agent such as LAH, SAH or NaBH₄ with ZnCl₂ or by catalytic hydrogenation. Cyclization of the amino alcohol with SOCl₂ or POCl₃ provides the 1-aryl-3-azabicyclo[3.1.0]hexane. The cyclization of substituted 4-aminobutan-1-ol by SOCl₂ or POCl₃ into the pyrrolidine ring system has been reported previously (Armarego et al., J. Chem. Soc. [Section C: Organic] 19:3222-9, 1971; and patent publication PL 120095 B2, CAN 99:158251).

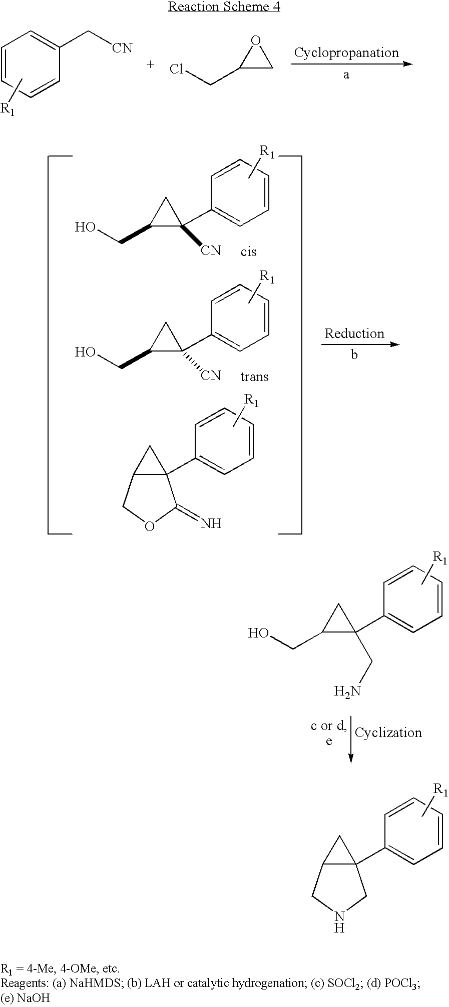

Reaction Scheme 5 provides an exemplary process for synthesizing the (1R,5S)-(+)-1-(4-methylphenyl)-3-azabicyclo[3.1.0]hexane hydrochloride or (+)-bicifadine and its chiral analogs. Using (S)-(+)-epichlorohydrin as a starting material in the same process described in Scheme 4 will ensure a final product with 1-R chirality (Cabadio et al., Fr. Bollettino Chimico Farmaceutico 117:331-42, 1978).

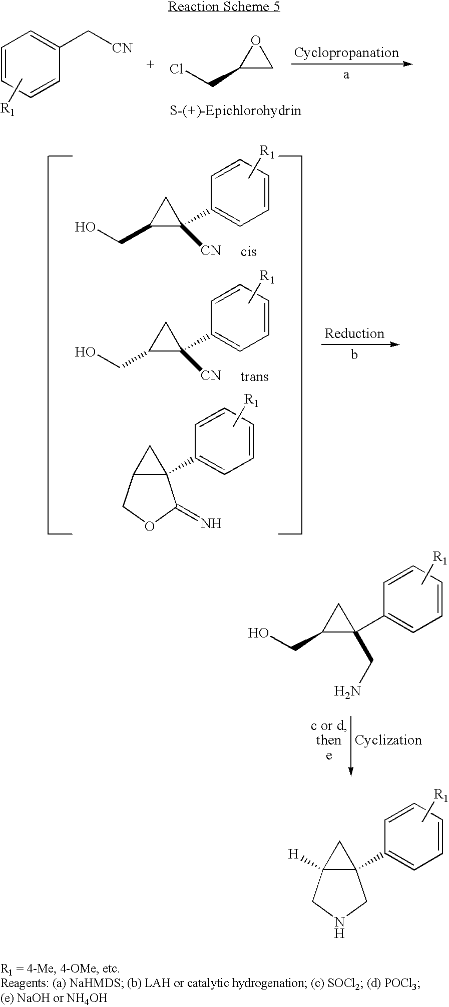

Reaction Scheme 6 provides an exemplary process to prepare the (1S,5R)-(−)-1-(4-methylphenyl)-3-azabicyclo[3.1.0]hexane hydrochloride or the (−)-bicifadine and its chiral analogs. Using (R)-(−)-epichlorohydrin as a starting material in the same process described in Scheme 4 will ensure a final product with 1-S chirality (Cabadio et al., Fr. Bollettino Chimico Farmaceutico 117:331-42, 1978).

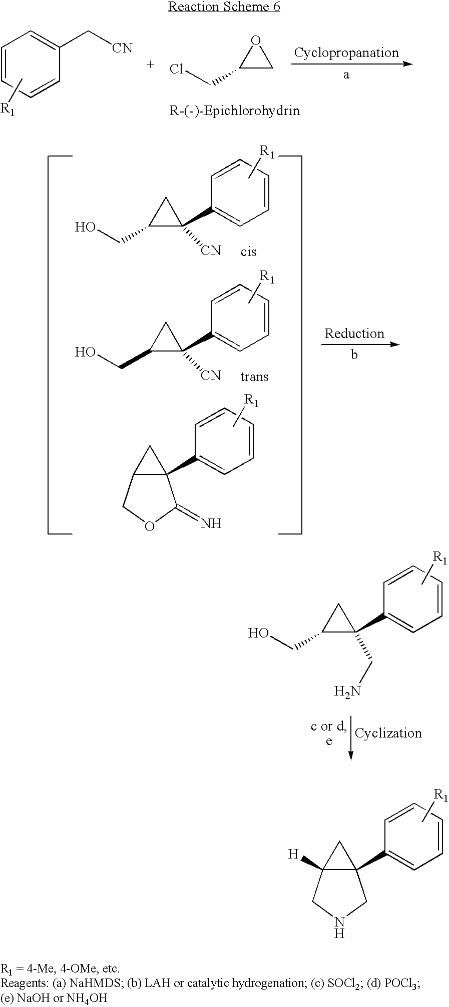

Reaction Scheme 7 provides an alternative exemplary process for transforming the 2-(hydroxymethyl)-1-arylcyclopropanecarbonitrile to a desired compound or intermediate of the invention via an oxidation and cyclization reaction. Utilizing chiral starting materials (+)-epichlorohydrin or (−)-epichlorohydrin will lead to the corresponding (+)- or (−)-bicifadine and corresponding chiral analogs through the same reaction sequences.

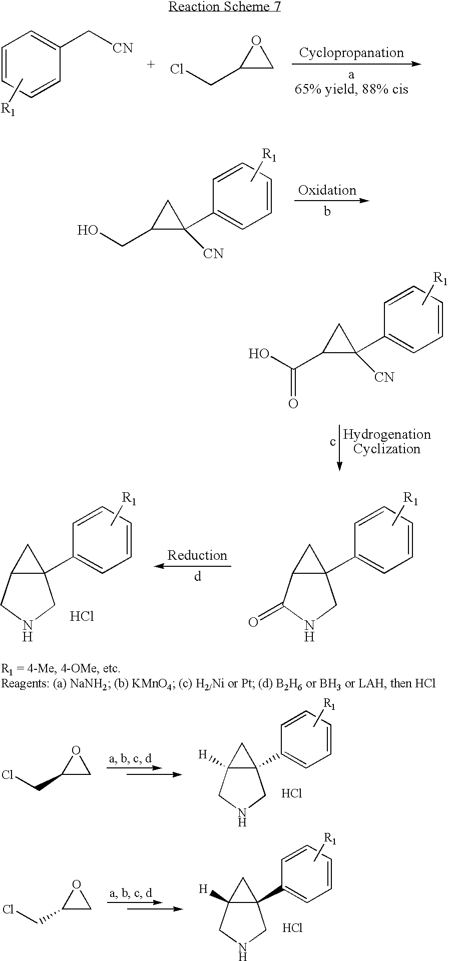

Reaction Scheme 8 provides an exemplary process for transforming the epichlorohydrin to a desired compound or intermediate of the invention via a replacement and cyclization reaction. The reaction of methyl 2-arylacetate with epichlorohydrin gives methyl 2-(hydroxymethyl)-1-arylcyclopropanecarboxylate with the desired cis isomer as the major product. The alcohol is converted into an OR₃ group such as —O-mesylate, —O-tosylate, —O-nosylate, —O-brosylate, —O-trifluoromethanesulfonate. Then OR₃ is replaced by a primary amine NH₂R₄, where R₄ is a nitrogen protection group such as a 3,4-dimethoxy-benzyl group or other known protection group. Nitrogen protecting groups are well known to those skilled in the art, see for example, “Nitrogen Protecting Groups in Organic Synthesis”, John Wiley and sons, New York, N.Y., 1981, Chapter 7; “Nitrogen Protecting Groups in Organic Chemistry”, Plenum Press, New York, N.Y., 1973, Chapter 2; See also, T. W. Green and P. G. M. Wuts in “Protective Groups in Organic Chemistry, 3rd edition” John Wiley & Sons, Inc. New York, N.Y., 1999. When the nitrogen protecting group is no longer needed, it may be removed by methods well known in the art. This replacement reaction is followed by a cyclization reaction which provides the amide, which is then reduced into an amine by a reducing agent such as LAH. Finally the protection group is removed to yield the bicifadine and other 1-aryl-3-azabicyclo[3.1.0]hexane analogs. Utilizing chiral (S)-(+)-epichlorohydrin as a starting material leads to the (1R,5S)-(+)-1-(4-methylphenyl)-3-azabicyclo[3.1.0]hexane hydrochloride or (+)-bicifadine and chiral 1-aryl-3-azabicyclo[3.1.0]hexane analogs with the same reaction sequence. Similarly, the (R)-(−)-epichlorohydrin will lead to the (1S,5R)-(−)-1-(4-methylphenyl)-3-azabicyclo[3.1.0]hexane hydrochloride or the (−)-bicifadine and chiral 1-aryl-3-azabicyclo[3.1.0]hexane analogs.

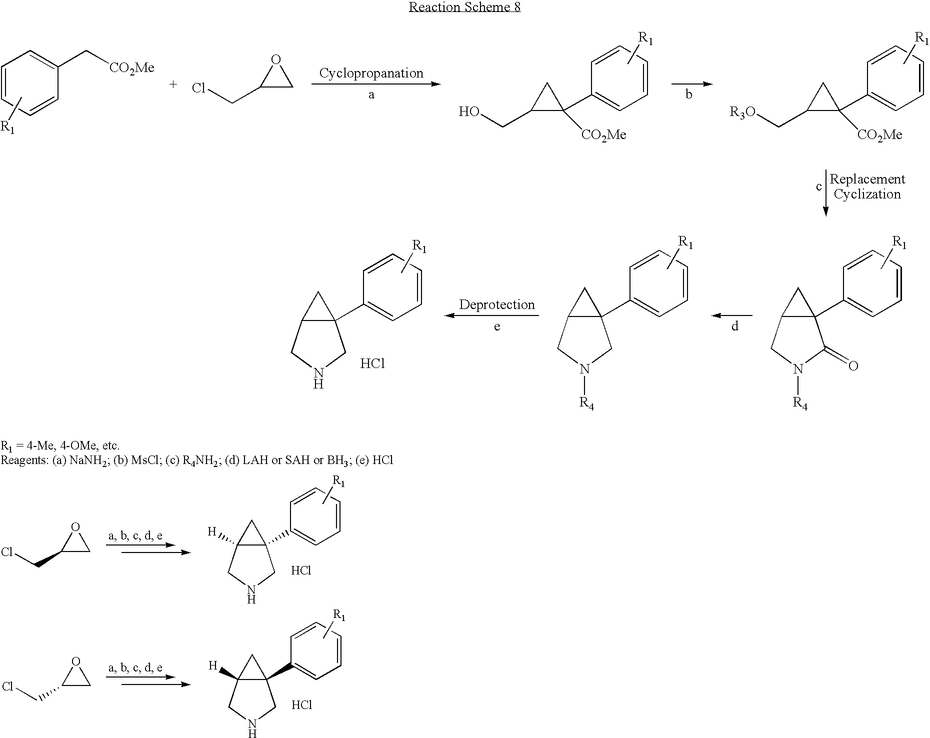

Reaction Scheme 9 provides an exemplary process for transforming the diol to a desired compound or intermediate of the invention. Reduction of the diester provides the diol which is then converted into an OR₃ group such as —O-mesylate, —O-tosylate, —O-nosylate, —O-brosylate, —O-trifluoromethanesulfonate. Then OR₃ is replaced by a primary amine NH₂R₆, where R₆ is a nitrogen protection group such as a 3,4-dimethoxy-benzyl group or other protection groups known in the art (e.g., allyl amine, tert-butyl amine). When the nitrogen protecting group is no longer needed, it may be removed by methods known to those skilled in the art.

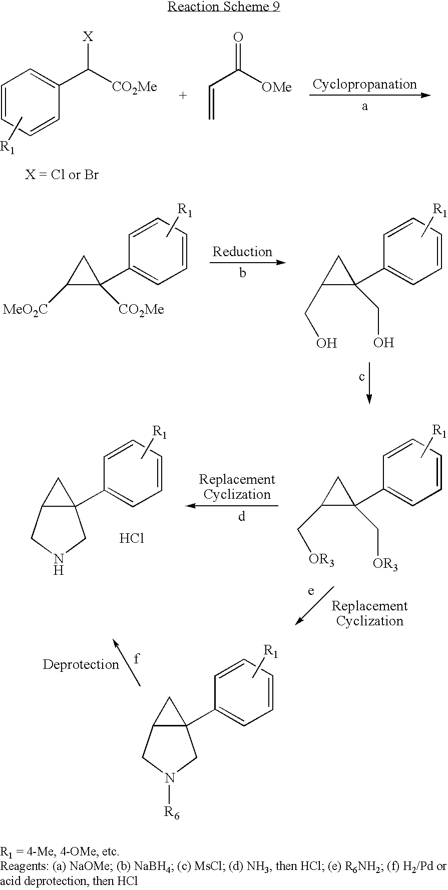

Reaction Scheme 10 provides an exemplary process for resolving the racemic 1-aryl-3-aza-bicyclo[3.1.0]hexane to enantiomers. The resolution of amines through tartaric salts is generally known to those skilled in the art. For example, using O,O-Dibenzoyl-2R,3R-Tartaric Acid (made by acylating L(+)-tartaric acid with benzoyl chloride) in dichloroethane/methanol/water, racemic methamphetamine can be resolved in 80-95% yield, with an optical purity of 85-98% (Synthetic Communications 29:4315-4319, 1999).

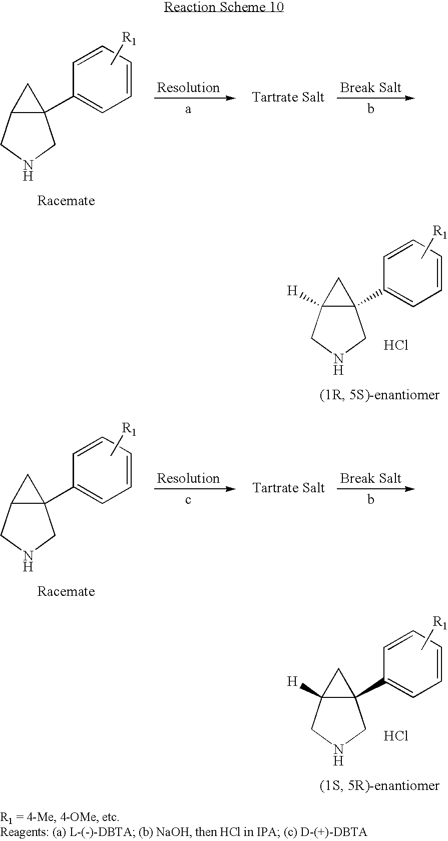

Reaction Scheme 1]provides an exemplary process for the preparation of 3-alkyl-1-aryl-3-azabicyclo[3.1.0]hexane analogs. These alkylation reactions reagents and conditons are generally well known to those skilled in the art.

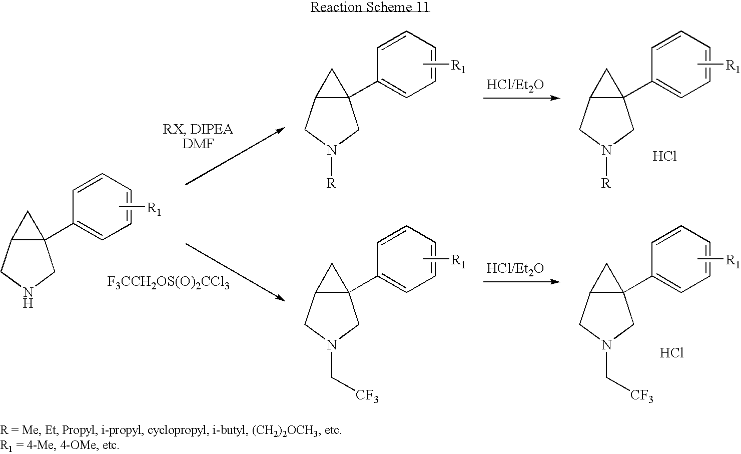

Enantiomers of compounds within the present invention can be prepared as shown in Reaction Scheme 12 by separation through a chiral chromatography.

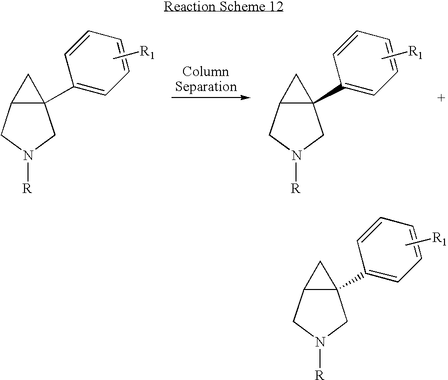

Alternatively, enantiomers of the compounds of the present invention can be prepared as shown in Reaction Scheme 13 using alkylation reaction conditions exemplified in scheme 11.

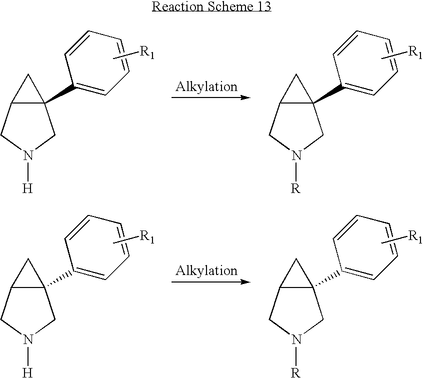

Reaction Scheme 14 provides an exemplary process for preparing some N-methyl 1-aryl-3-aza-bicyclo[3.1.0]hexane analogs. The common intermediate N-methyl bromomaleide is synthesized in one batch followed by Suzuki couplings with the various substituted aryl boronic acids. Cyclopropanations are then carried out to produce the imides, which are then reduced by borane to provide the desired compounds. 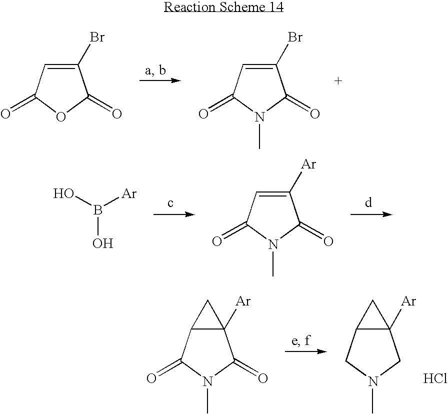
Ar=4-(trifluoromethyl)phenyl, 3-chlorophenyl, 4-fluorophenyl, 4-cyanophenyl (before step e) or 4-aminomethylphenyl(after step e), etc.

Reagents and conditions: (a) MeNH₂, THF, 10° C., 1.5 hr; (b) NaOAc, Ac₂O, 60° C., 2 hr; (c) PdCl₂(dppf), CsF, dioxane, 40° C., 1-6 hr; (d) Me₃SOCl, NaH, THF, 50-65° C., 2-6 hr; (e) 1 M BH₃/THF, 0° C.; 60° C. 2 hr (f) HCl, Et₂O

Reaction Scheme 15 provides an additional methodology for producing 1-aryl-3-azabicyclo[3.1.0]hexanes.

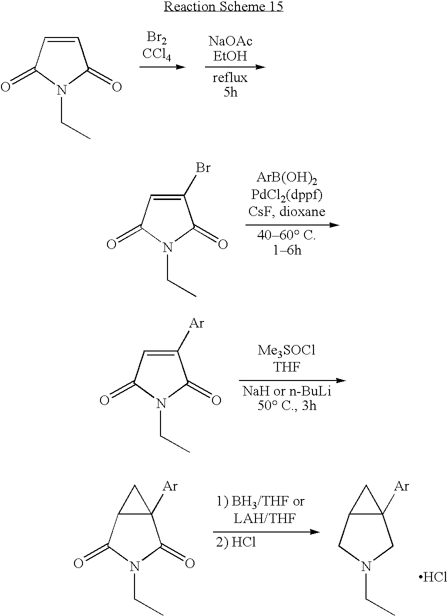

Reaction Scheme 16 provides an additional methodology for producing 1-aryl-3-azabicyclo[3.1.0]hexanes.

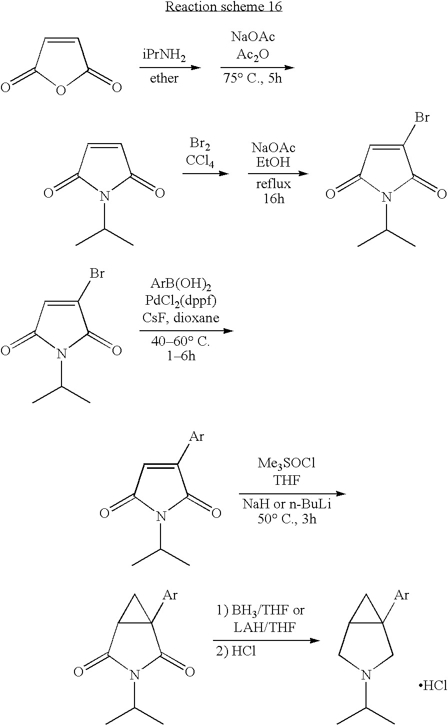

Reaction Scheme 17 provides an additional methodology for producing 1-aryl-3-azabicyclo[3.1.0]hexanes.

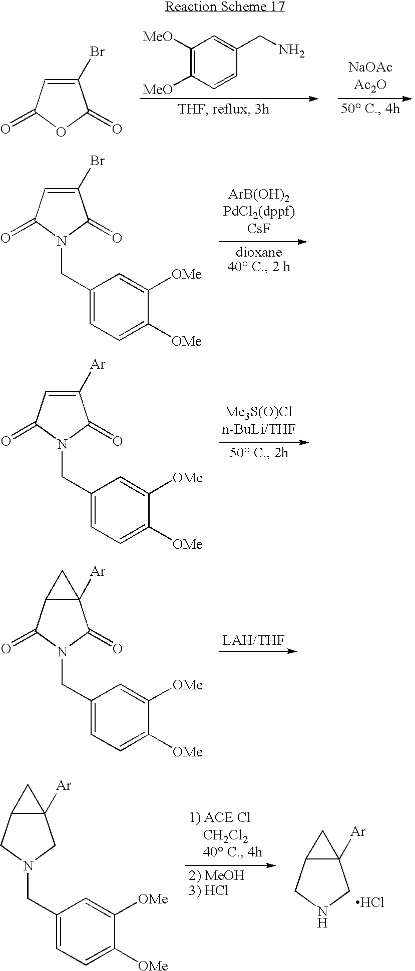

Reaction Scheme 18 provides an additional methodology for producing 1-aryl-3-azabicyclo[3.1.0]hexanes. Utilizing chiral starting materials (+)-epichlorohydrin or (−)-epichlorohydrin will lead to the corresponding chiral analogs through the same reaction sequences.

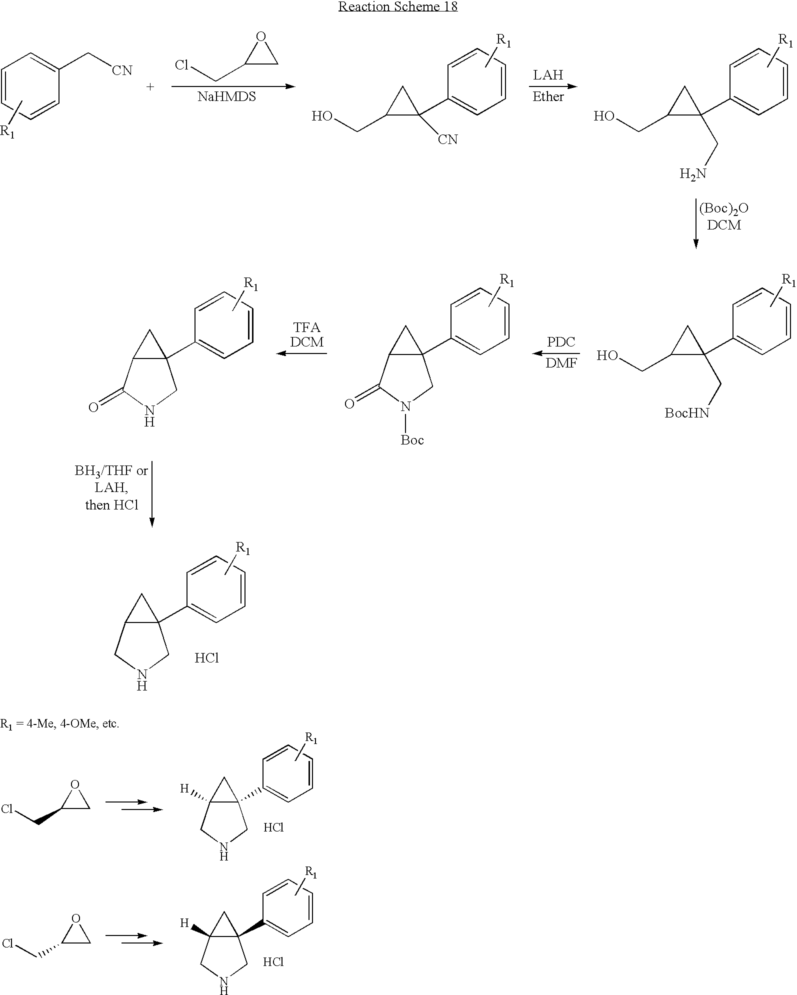

### EXAMPLE II

### Preparation of 1-p-tolyl-3-aza-bicyclo[3.1.0]hexane hydrochloride using Reaction Scheme 4

### A. Synthesis of 2-(hydroxymethyl)-1-p-tolylcyclopropanecarbonitrile

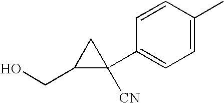

Method 1

To a stirring solution of p-tolylacetonitrile (16.8 g, 0.128 moles) in anhydrous THF (250 mL) at −18° C. under nitrogen, was added 128 mL of sodium bis (trimethylsilyl)amide (NaHMDS, 1M in THF) slowly via addition funnel while keeping the temperature below 10° C. The resulting brown mixture was stirred for 0.5 h between −10° C. and −20° C. Epichlorohydrin (11.8 g, 0.128 moles in 20 mL of THF) was added slowly over 15 minutes while keeping the temperature below −10° C. The mixture was stirred between −10° C. and −20° C. for 0.5 h then NaHMDS (128 mL, 0.191 moles) was added while keeping the temperature between −15° C. and −20° C. The mixture was stirred for 45 minutes then quenched with 200 mL of water. The mixture was stirred 5 minutes, allowed to settle and the layers were separated. The lower aqueous layer was re-extracted with EtOAc (2×250 mL). The organics were combined, washed with 100 mL of 1M HCl, 100 mL of saturated NaCl, dried over Na₂SO₄, filtered and concentrated to provide 24 g of crude product as an orange oil. Chromatography through a short silica gel plug eluted with EtOAc/Heptane (5-50%) afforded 8.9 g (39% yield) of product as an orange oil. The ¹H NMR indicated a 3.5:1 ratio of cis to trans isomers. LC/MS (m/z M⁺188); ¹³C NMR (CDCl₃)) δ 16.28, 18.28, 19.13, 21.21, 21.33, 21.37, 29.83, 31.48, 61.14, 63.36, 121.17, 126.35, 129.39, 129.83, 130.01, 132.93, 137.95; Z-diastereomer ¹H NMR CDCl₃ (400 MHz) δ 1.57 (m, 2H, ArCCH₂CH), 1.90 (m, 1H, ArCCH₂CH), 2.11 (m, 1H, ArCCH₂CH), 2.35 (s, 3H, CH₃), 3.81 (dd, 1H, CHOH, J=12.1 Hz, J=8.3 Hz), 4.08 (dd, 1H, CHOH, J=12.1 Hz, J=5.3 Hz), 7.20 (m, 4H, ArH); E-diastereomer ¹H NMR CDCl₃ (400 MHz) δ 1.48 (dd, 1H, ArCCH₂CH, J=7.0 Hz, J=5.9 Hz), 1.72 (dd, 1H, ArCCH₂CH, J=9.4 Hz, J=5.8 Hz), 2.33 (s, 3H, CH₃), 3.15 (dd, 1H, CHOH, J=12.1 Hz, J=8.3 Hz), 3.51 (dd, 1H, CHOH, J=12.1 Hz, J=5.3 Hz), 7.20 (m, 4H, ArH).

Method 2

A flask was charged with 200 mL THF, 20.00 g (152.4 mmol) of p-tolyl acetonitrile, 15.51 g (167.7 mmol, 1.1 equiv) epichlorohydrin and 50 ml of N,N′-DMPU. The colorless mixture was cooled to −35° C. (inside temperature) under stirring. Then, 335 mL (335 mmol, 2.2 equiv) of a 1 M THF solution of sodium bis-(trimethylsilyl)amide were added dropwise keeping the inside temperature between −35° C. and −20° C. The mixture turned to a yellowish then to an orange color (time of addition: about 15 min). After full addition the mixture was stirred another 4 h at −25° C. (±5° C.) for full conversion (HPLC-control). Then, the mixture was cooled to −60° C. At this temperature 176 mL (704 mmol, 4.6 equiv) of a 4 M solution of HCl in dioxane were added within 30 min, keeping the temperature between −60° C. and −55° C. The mixture was then allowed to warm to room temperature within 1.5 h under stirring.

The mixture was taken in tert-butyl methyl ether (300 mL) and extracted three (3) times with water (3×200 mL). The combined aqueous layer was reextracted with tert-butyl methyl ether (300 mL). The combined organic layer was dried over sodium sulphate, filtered and concentrated on rotavap (20 mbar) then in high vacuum to obtain 29.48 g (103% crude yield) of an orange crude oil. The NMR and HPLC spectra of the crude material show about a 6.5:1 ratio of Z to E-isomer. The HPLC purity of Z+E was ca. 96 area % @220 nm. Some 2% of p-tolyl acetonitrile was still present in the crude material. ¹H NMR (CDCl₃, 300 MHz) cis-isomer δ 7.22-7.14 (m, 4H, ArH), 4.08 (dd, J=12.0, 5.1 Hz, 1H, CH₂OH), 3.82 (m, 1H, CH₂OH), 2.34 (s, 3H), 1.90 (m, 1H, ArCCH₂CH), 1.58 (m, 2H, ArCCH₂CH); trans-isomer: δ 7.29-7.20 (m, 4H, ArH), 3.52 (dd, J=12.4, 5.3 Hz, 1H, CH₂OH), 3.15 (dd, J=12.4, 8.4 Hz, 1H, CH₂OH), 2.35 (s, 3H), 2.15 (m, 1H, ArCCH₂CH), 1.72 (dd, J=9.4, 5.9 Hz, 1H, ArCCH₂CH), 1.50 (dd, J=7.0, 5.9 Hz, 1H, ArCCH₂CH).

### B. Synthesis of (2-(Aminomethyl)-2-p-tolylcyclopropyl)methanol

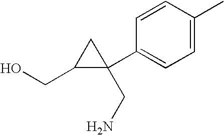

Method 1

To a stirring slurry of lithium aluminum hydride (LAH) (4.3 g, 0.114 moles) in diethyl ether (300 mL) at 0-5° C. was added a solution of crude nitrile (10.7 g, 0.057 moles) in 100 mL of Et₂O, slowly via addition funnel while keeping the temperature below 10° C. The mixture was stirred for 45 minutes after which time, no starting material was observed by TLC analysis (SiO₂ plate, EtOAc/Heptane 1:1). The reaction was carefully quenched by the dropwise addition of H₂O (4 mL) followed by 4 mL of 15% NaOH and lastly 12 mL of H₂O. The resulting off-white slurry was stirred for 0.5 h then filtered through a Celite pad, washing with 2×250 mL of Et₂O. The filtrate was concentrated to give 11.4 g of crude product as a pale yellow oil. Chromatography on silica gel eluting with CH₂Cl₂/MeOH/NH₄OH (20:1:0.1 to 10:1:0.1) afforded 6.6 g (60%) of pure amino alcohol as colorless oil. ¹H NMR CDCl₃ (400 MHz) δ 0.71 (m, 1H, ArCCH₂CH), 0.92 (dd, 1H, ArCCH₂CH J₁=8.6 Hz, J₂=4.7 Hz), 1.72 (m, 1H, ArCCH₂CH), 2.32 (s, 3H, CH₃), 2.56 (d, 1H, CH, J=12.5 Hz), 2.80 (br. s, 3H, NH₂, OH), 3.33 (dd, 1H, CHOH, J=12.3 Hz, J=10.9 Hz), 3.42 (dd, 1H, CHN, J=12.4 Hz, J=0.9 Hz), 4.11 (dd, 1H, CHOH, J=12.3 Hz, J=5.5 Hz), 7.12 (m, 2H, ArH), 7.28 (m, 2H, ArH); ¹³C NMR (CDCl₃) δ 18.61, 21.24, 25.37, 31.45, 47.05, 63.88, 129.47, 129.73, 136.73, 141.25; LC/MS (m/z M⁺ 192).

Method 2

An autoclave was charged with 570 mL methanol (saturated with dry ammonia), 29.48 g (152.4 mmol) of crude nitrile and 15.0 g of RaCo SK03/06 (prewashed with methanol). The autoclave was closed and purged three times with nitrogen (10 bar), then three times with hydrogen (10 bar). The heating was switched on and when the temperature reached 80° C. the pressure was set to 50 bar (725 psi) and stirring was started. After 24 h, the autoclave was cooled to room temperature and the pressure was released.

The mixture was filtered over a short pad of Hyflo and the filtrate was concentrated under reduced pressure on rotavap (20 mbar) then in high vacuum to obtain 29.15 g of a brownish crude oil. This crude material was taken in 150 ml 2 M aqueous HCl. The aqueous layer was washed twice with 100 ml dichloromethane. The combined organic layer was reextracted with 0.1 M aqueous HCl. The dichloromethane layer from the washing was put aside. The combined aqueous layer was basified with aqueous 25% ammonia to pH=8 and reextracted twice with 100 ml dichloromethane. The combined organic layer was washed with aqueous 2% ammonia, dried over sodium sulphate and concentrated under reduced pressure on rotavap (20 mbar) then in high vacuum to afford 24.48 g of a light-brown oil. ¹H NMR (CDCl₃, 300 MHz) δ 7.32-7.12 (m, 4H, ArH), 4.12 (dd, J=12.2, 5.5 Hz, 1H), 3.46-3.30 (m, 2H), 2.90 (bs, 3H), 2.58 (d, J=12.2 Hz, 1H), 2.33 (s, 3H), 1.72 (m, 1H, ArCCH₂CH), 0.94 (dd, J=8.6, 4.7 Hz, 1H, ArCCH₂CH), 0.72 (m, 1H, ArCCH₂CH).

The material was dissolved in 30 ml acetonitrile. 26 ml HCl (6 M in 2-propanol) was added followed by about 150 ml of diethyl ether. Crystal germs of pure Z-isomer (HCl salt) were added to the cloudy mixture. The crystals were filtered off and additional material was obtained from the mother liquor by adding another 30 ml of diethyl ether. The combined crystals were washed with diethyl ether/acetonitrile (5:2) and diethyl ether and dried in high vacuum to obtain 20.61 g (59% yield) of the target compound HCl salt as creme colored crystals. The NMR and HPLC spectra of the crystals showed a ca. 97% chemical purity of desired Z-isomer. About 3% of E-isomer impurity was present in the crystals. ¹H NMR (D₆-DMSO, 300 MHz) δ 7.88 (bs, 3H, NH₃Cl), 7.29-7.14 (m, 4H, ArH), 5.25 (bs, 1H, OH), 3.87 (dd, J=12.0, 5.4 Hz, 1H), 3.42-3.12 (m, 3H), 2.28 (s, 3H), 1.37 (m, 1H, ArCCH₂CH), 1.04 (m, 1H, ArCCH₂CH), 0.94 (m, 1H, ArCCH₂CH).

### C. Synthesis of 1-p-Tolyl-3-aza-bicyclo[3.1.0]hexane hydrochloride

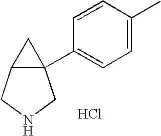

Method 1

Pursuant to step c of Reaction Scheme 4, to a stirring solution of the amino alcohol (5.18 g, 0.027 moles) in 50 mL of dichloroethane (DCE), at 0° C. under nitrogen, was added 2.6 mL (0.035 moles, 1.3 eq) of SOCl₂ slowly via syringe while keeping the temperature below 50° C. (Note: The reaction exotherms from 22° C. to 46° C.) The resulting mixture was stirred for 3.5 h at room temperature after which time, TLC analysis (SiO₂ plate, CH₂Cl₂/MeOH/NH₄OH (10:1:0.1)) showed no remaining starting material. The mixture was quenched with 50 mL of water and the layers were separated. The organic layer was washed with H₂O (2×75 mL). The aqueous layers were combined, basified with 10N NaOH to pH=10 (pH paper) and extracted with 3×75 mL of CH₂Cl₂. The combined organics were dried over Na₂SO₄, filtered and concentrated to a dark oil. The oil was dissolved in MeOH (20 mL), treated with 15 mL of 2M HCl/Et₂O and concentrated in vacuo to a semi solid. Acetonitrile (−50 mL) was added at ambient temperature. The resulting slurry was filtered and the product cake was washed with 2×20 mL of CH₃CN. The product was dried overnight (˜29 mmHg, 50° C.) to give 3.68 g (65%) of pure product as a white solid. mp=205-207° C.; ¹H NMR CDCl₃ (400 MHz) δ 1.20 (m, 1H, ArCCH₂CH), 1.52 (m, 1H, ArCCH₂CH), 1.91 (m, 1H, ArCCH₂CH), 2.31 (s, 3H, CH₃), 3.60 (m, 3H, CH₂N, CHN), 3.74 (m, 1H, CHN), 7.10 (m, 4H, ArH), 9.74 (br. s, 1H, NH), 10.23 (br.s, 1H, NHCl); ¹³C NMR (CDCl₃)) δ 15.4, 21.2, 23.2, 31.3, 47.9, 51.1, 127.4, 126.0, 129.7, 135.1, 137.3; LC/MS (m/z M⁺ 174) Reverse Phase HPLC retention time=5.31 min.

Method 2

Pursuant to step d of Reaction Scheme 4, to a stirring solution of amino alcohol (574 mg, 3 mmoles) in 3 mL of DCE, at room temperature under nitrogen, was added 2 mL (21.4 mmoles, 7 eq) of POCl₃ slowly via syringe while keeping the temperature below 45° C. (Note: The reaction exotherms slightly and turns darker in color) The resulting mixture was stirred for ˜18 h (overnight) at ambient temperature. The mixture was quenched with 15 mL of water. The mixture was basified to pH=10 with 25% NaOH and extracted with CH₂Cl₂ (2×100 mL). The combined organics were washed with saturated NaCl, dried over Na₂SO₄, filtered and concentrated to a dark oil. Chromatography on a silica gel pad eluting with CH₂Cl₂/MeOH/NH₄OH (20:1:0.1 to 10:1:0.1) provided 130 mg (26%) of product as a glassy semi-solid. The product still appears to contain a small amount of impurity by ¹H NMR. ¹H NMR CDCl₃ (400 MHz, partial assignment), δ 1.18 (m, 1H), 1.50 (m, 1H), 1.91 (m,1H), 2.32 (s, 3H), 3.59 (m, 3H), 3.73-3.76 (d,12H, J=11 Hz), 3.20 (d, 1H), 7.08 (m, 4H).

Method 3

Pursuant to step c of Reaction Scheme 4, a flask was charged with 350 mL of toluene. 18.30 g (80.37 mmol) of the amino alcohol HCl salt were added and the stirred white suspension was externally cooled with an ice/2-propanol-bath. Then, 7.00 mL (96.44 mmol, 1.2 equiv) of thionyl chloride were added dropwise. A small exotherm could be detected (the inside temperature rose from 0 to 4° C.). After full addition the mixture was stirred 2.5 h at this temperature (0-3° C.). The initial suspension turned almost completely homogeneous. Then 120 mL (482.20 mmol, 6.0 equiv) of a 4 M aqueous NaOH solution were added within 45 min keeping the inside temperature below 5° C. with external cooling. The now white emulsion was allowed to warm to room temperature and stirred overnight.

The aqueous layer was separated and reextracted with 100 mL toluene. The combined toluene layer was dried over sodium sulphate and concentrated on rotavap (20 mbar) then in high vacuum to afford 14.85 g (107% crude yield) of a clear yellowish oil. The HPLC purity of the material was about 96 area % @220 nm. ¹H NMR (CDCl₃, 300 MHz) δ 7.11 (m, 4H, ArH), 3.25-3.00 (m, 4H, CH₂NCH₂), 2.32 (s, 3H), 1.85 (bs, 1H, NH), 1.66 (m, 1H, ArCCH₂CH), 0.92 (m, 1H, ArCCH₂CH), 0.81 (m, 1H, ArCCH₂CH).

The crude material was dissolved in 40 ml ethyl acetate. About 200 mg of insoluble white solid were removed with filtration. To the filtrate was added 18 mL (90 mmol, 1.1 equiv) of HCl/2-propanol under stirring at room temperature. Bicifadine HCl crystallized immediately from the mixture. The mixture was further diluted with 20 mL of ethyl acetate and stirred another 10 min at room temperature. The white crystals were filtered to obtain 12.58 g (75% yield) of pure product. The NMR and HPLC spectra of the crystals show a >98% chemical purity.

### EXAMPLE III

### Preparation of (1R,5S)-(+)-1-p-Tolyl-3-azabicyclo[3.1.0]hexane Hydrochloride Using Reaction Scheme 5

### A. Synthesis of (1R,2S)-2-(Hydroxymethyl)-1-p-tolylcyclopropanecarbonitrile

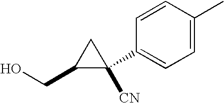

Method 1

To a stirring solution of p-tolylacetonitrile (25.1 g, 0.191 moles) in THF (250 mL) at 0° C. under nitrogen, was added 191 mL of NaHMDS (1M in THF) slowly via addition funnel while keeping the temperature below 10° C. The resulting brown mixture was stirred for 0.5 h at 5-10° C. A solution of S-(+)-epichlorohydrin (17.7 g, 0.191 moles) in 20 mL of THF was added slowly over 15 minutes while keeping the temperature below 20° C. The mixture was stirred between 10° C. and 20° C. for 0.5 h then cooled to 0-5° C. and NaHMDS (191 mL, 0.191 moles) was added while keeping the temperature between 5° C. and 10° C. The mixture was stirred for 45 minutes then quenched with 200 mL of water. The mixture was stirred 5 minutes, allowed to settle and the layers were separated. The lower aqueous layer was re-extracted with EtOAc (2×250 mL). The organics were combined, washed with saturated NaCl, dried over Na₂SO₄, filtered and concentrated to an orange oil. Chromatography through a short silica gel plug eluted with EtOAc/Heptane (5-50%) afforded 19.6 g (55%) of product as an orange oil. The ¹H NMR indicated about a 2.8:1 ratio of cis to trans isomers. Z-diastereomer ¹H NMR CDCl₃ (400 MHz partial assignment) δ 1.57 (m, 2H), 1.91 (m, 1H), 2.11 (m, 1H), 2.35 (s, 3H), 3.81 (m, 1H), 4.08 (m, 1H), 7.20 (m, 4H); E-diastereomer ¹H NMR CDCl₃ (400 MHz partial assignment)) δ 1.48 (m, 1H), 1.71 (m, 1H)), 2.33 (s, 3H), 3.14 (m, 1H), 3.51 (m, 1H), 7.20 (m, 4H).

Method 2

A flask was charged with 1.5 liters of THF, 200.0 g (1524.4 mmol) of p-tolyl acetonitrile and 155.5 g (1680.5 mmol, 1.1 equiv) (s)-epichlorohydrin. The colorless mixture was cooled to −28° C. (inside temperature) under stirring. Then, 1.68 liters (3360.0 mmol, 2.2 equiv) of a 2 M THF solution of sodium bis-(trimethylsilyl)amide were added dropwise keeping the inside temperature between −28° C. and −20° C. The mixture turns to a yellowish then orange color (time of addition: ca. 20 min). After full addition the mixture was stirred another 4 h at −25° C. (±2° C.) for full conversion (HPLC-control). Then, the mixture was cooled to −60° C. At this temperature 1.75 liters (7012.2 mmol, 4.6 equiv) of a 4 M solution of HCl in dioxane were added within 140 min keeping the inside temperature between −60° C. and −55° C. The mixture was then allowed to warm to room temperature under stirring.

The mixture was taken in tert-butyl methyl ether (2 L) and water (1.5 L). The water layer was separated and the organic layer washed with water (2×2 L). The combined aqueous layer was reexttracted with in tert-butyl methyl ether (1.2 L). The combined organic layer was dried over sodium sulphate, filtered and concentrated on rotavap (20 mbar) then in high vacuum to afford 306.5 g (107% crude yield) of an orange crude oil.

The NMR and HPLC spectra of the crude material show a ca. 6.2:1 ratio of Z to E-isomer. The HPLC purity of Z+E was ca. 96 area % @220 nm.

### B. Synthesis of ((1S,2R)-2-(Aminomethyl)-2-p-tolylcyclopropyl)methanol

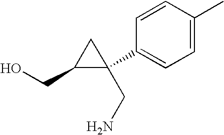

Method 1

Pursuant to step b of Reaction Scheme 5, to a stirring slurry of LAH (7.7 g, 0.205 moles) in diethyl ether at ambient temperature was added a solution of the crude nitrile desribed in this Example III, section A, method 1 (19.6 g, 0.102 moles) in 100 mL of Et₂O, slowly via addition funnel while keeping the temperature below 30° C. The mixture was stirred at room temperature for 1 h after which time, no starting nitrile was observed by TLC analysis (SiO₂ plate, EtOAc/Heptane 1:1). The reaction was allowed to stir for an additional 0.5 h then carefully quenched by the dropwise addition of H₂O (8 mL) followed by 8 mL of 25% NaOH and lastly 24 mL of H₂O. The resulting off white slurry was stirred for ˜1 h then filtered through a Celite pad, washing with Et₂O (3×250 mL). The filtrate was concentrated to give 20.8 g of crude product as a pale yellow oil. The product was carried forward crude without further purification. Crude ¹H NMR CDCl₃ (400 MHz, partial assignment), δ 0.71 (m, 1H), 0.92 (dd, 1H), 1.72 (m, 1H), 2.32 (s, 3H, CH₃), 2.54-2.57 (d, 1H, J=12.4 Hz), 3.33 (m, 1H), 4.09-4.13 (dd, 1H, J=12.3 Hz, J=1 Hz), 7.12 (m, 2H), 7.28 (m, 2H).

Method 2

An autoclave was charged with 3.0 L methanol, 450 g ammonia (liquid), 304.3 g (1513.3 mmol) of the crude product (described in section A, method 2 of this example) and 152 g of RaCo SK03/06 (prewashed with methanol). The autoclave was closed and purged three times with nitrogen (10 bar), then three times with hydrogen (10 bar). The heating was switched on and when the temperature reached 80° C. the pressure was set to 50 bar (725 psi) and stirring was started. After 20 h, the autoclave was cooled to room temperature and the pressure was released.

The mixture was filtered over a short pad of Hyflo and the filtrate was concentrated under reduced pressure on rotavap (20 mbar) then in high vacuum to obtain 311.0 g of an brownish crude oil. This crude material was taken in 1.2 liters of 2 M aqueous HCl. The aqueous layer was washed twice with dichloromethane (1×500 ml, 1×200 ml). The combined organic layer was reextracted with 0.2 M aqueous HCl (300 ml). The dichloromethane layer from the washing was put aside. The combined aqueous layer was basified with aqueous 25% ammonia to pH=8 and reextracted with dichloromethane (1×11, 2×250 ml). The combined organic layer was dried over sodium sulphate and concentrated under reduced pressure on rotavap (20 mbar) then in high vacuum to afford 251.7 g of a light-brown oil.

The material was dissolved in 320 ml acetonitrile. 250 ml HCl (6 M in 2-propanol) was added followed by ca. 1.1 l of diethyl ether. Previously prepared crystal germs of pure Z-isomer product (HCl salt) were added to the cloudy mixture. After a first crystallization more material was obtained by adding another 200 ml of diethyl ether. The crystals were filtered off and washed with 2×120 ml diethyl ether/acetonitrile (1:1) and diethyl ether (2×100 ml) and dried in high vacuum (70° C., 2 h) to afford 159.5 g (46% yield) of the title compound as HCl salt as white crystals. The NMR and HPLC spectra of the crystals showed a ca. 98% chemical purity of desired Z-isomer. Ca. 1% of E-isomer impurity was present in the crystals.

Free base ¹H NMR (CDCl₃, 300 MHz) δ 7.32-7.12 (m, 4H, ArH), 4.12 (dd, J=12.2, 5.5 Hz, 1H), 3.46-3.30 (m, 2H), 3.02 (bs, 3H), 2.58 (d, J=12.2 Hz, 1H), 2.33 (s, 3H), 1.72 (m, 1H, ArCCH₂CH), 0.94 (dd, J=8.6, 4.7 Hz, 1H, ArCCH₂CH), 0.72 (m, 1H, ArCCH₂CH). HCl salt ¹H NMR (D₆-DMSO, 300 MHz) δ 7.88 (bs, 3H, NH₃Cl), 7.29-7.14 (m, 4H, ArH), 5.25 (bs, 1H, OH), 3.87 (dd, J=12.0, 5.4 Hz, 1H, CH₂OH), 3.42-3.12 (m, 3H, CH₂OH, CH₂N), 2.28 (s, 3H), 1.37 (m, 1H, ArCCH₂CH), 1.04 (m, 1H, ArCCH₂CH), 0.94 (m, 1H, ArCCH₂CH).

### C. Synthesis of (1R,5S)-(+)-1-p-Tolyl-3-azabicyclo[3.1.0]hexane Hydrochloride

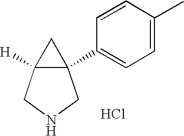

Method 1

Pursuant to step c of Reaction Scheme 5, to a stirring solution of crude amino alcohol (20.6 g, 0.108 moles) in 200 mL of DCE, at room temperature under nitrogen, was added 9.4 mL (0.129 moles, 1.2 eq) of SOCl₂ slowly via syringe while keeping the temperature below 45° C. (Note: The reaction exotherms from 22° C. to 40° C.) The resulting mixture was stirred for 3.5 h at ambient temperature after which time, TLC analysis (SiO₂ plate, CH₂Cl₂/MeOH/NH₄OH (10:1:0.1)) showed no starting material. The mixture was quenched with 75 mL of water and the layers were separated. The organic layer was washed with 2×100 mL of H₂O. The aqueous layers were combined, basified with 10N NaOH to pH=10 (pH paper) and the cloudy mixture was extracted with 2×150 mL of CH₂Cl₂. The combined organics were dried over Na₂SO₄, filtered and concentrated to a dark oil. The oil was dissolved in MeOH (40 mL) and treated with 55 mL of 2M HCl/Et₂O. The mixture was concentrated to approximately one fourth of the original volume, diluted with CH₃CN (75 mL) and further concentrated to a slurry. Acetonitrile (75 mL) was added, the mixture was heated to a gentle reflux (75-80° C.) for 1 minute then allowed to cool to room temperature. The resulting slurry was filtered and the product cake was washed with CH₃CN (2×50 mL). The product was dried 6 h (29 mmHg, 50° C.) to give 6.5 g (16% from p-tolylacetonitrile) of pure product as a white solid. mp=207-209° C.; ¹H NMR CDCl₃ (400 MHz) δ 1.20 (m, 1H, ArCCH₂CH), 1.52 (m, 1H, ArCCH₂CH), 1.91 (m, 1H, ArCCH₂CH), 2.31 (s, 3H, CH₃), 3.60 (m, 3H, CH₂N, CHN), 3.74 (m, 1H, CHN), 7.10 (m, 4H, ArH), 9.74 (br. s, 1H, NH), 10.23 (br.s, 1H, NHCl) ¹³C NMR (CDCl₃) δ 15.4, 21.2, 23.2, 31.3, 47.9, 51.1, 127.4, 129.7, 135.1, 137.3; LC/MS (m/z M⁺ 174); Reverse Phase HPLC retention time=5.55 min; Normal Phase Chiral HPLC retention time=7.27 min; [α]²⁰_{D}=+65 (c=1, MeOH).

Method 2

Pursuant to step c of Reaction Scheme 5, a flask was charged with 2.0 L ethyl acetate. 157.3 g (690.8 mmol) of the HCl salt (described in section B, method 2 in this example) were added and the stirred white suspension was externally cooled with an ice/2-propanol-bath. 60.3 ml (828.9 mmol, 1.2 equiv) of thionyl chloride were added dropwise at 0° C. within 20 min. A small exotherm could be detected (the inside temperature rose from 0 to 3° C.). After full addition the mixture was stirred 1.5 h at low temperature (0-3° C.). The initial suspension turned almost completely homogeneous. Then 824 ml (5526.0 mmol, 8.0 equiv) of 12.5% ammonium hydroxide were added within 60 min keeping the inside temperature below 5° C. with external cooling. The now white emulsion was allowed to warm to room temperature and stirred overnight. The aqueous layer was separated and reextracted with 500 ml ethyl acetate. The combined ethyl acetate layer was dried over sodium sulphate and concentrated on rotavap (20 mbar) then in high vacuum to afford 129.0 g (108% crude yield) of a clear yellowish oil. The HPLC purity of the material is ca. 97 area % @220 nm.

The crude material was dissolved in 550 ml ethyl acetate. To the filtrate was added 1.0 equiv of HCl/2-propanol under stirring at room temperature. Bicifadine HCl crystallized immediately from the mixture with low exotherms. The white crystals were filtered to afford 126.2 g (87% yield) of (+)-Bicifadine HCl. The NMR and HPLC spectra of the crystals show a >98% chemical purity. The material has >98% ee as measured by chiral HPLC. [α]²⁰_{D}=+62 (c=1, MeOH). ¹H NMR (D₆-DMSO, 300 MHz) δ 9.90 (bs, 2H, NH₂Cl), 7.15-7.07 (m, 4H, ArH), 3.78-3.56 (m, 4H, CH₂NCH₂), 1.92 (m, 1H, ArCCH₂CH), 1.54 (dd, J=6.6, 4.8 Hz, 1H, ArCCH₂CH), 1.20 (m, 1H, ArCCH₂CH).

### EXAMPLE IV

### Preparation of (1S,5R)-(−)-1-p-Tolyl-3-azabicyclo[3.1.0]hexane Hydrochloride Using Reaction Scheme 6

### A. Synthesis of (1S,2R)-2-Hydroxymethyl-1-p-tolyl-cyclopropancarbonitrile

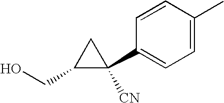

Method 1

To a stirring solution of p-tolylacetonitrile (25 g, 0.191 moles) in THF (250 mL) at 0° C. under nitrogen, was added 191 mL of NaHMDS (1M in THF) slowly via addition funnel while keeping the temperature below 10° C. The resulting brown mixture was stirred for 0.5 h at 5-10° C. A solution of R-(−)-Epichlorohydrin (17.7 g, 0.191 moles) in 20 mL of THF was added slowly over 15 minutes while keeping the temperature below 20° C. The mixture was stirred between 10° C. and 20° C. for 0.5 h then cooled to 0-5° C. and NaHMDS (191 mL, 0.191 moles) was added while keeping the temperature between 5° C. and 10° C. The mixture was stirred for 45 minutes then quenched with 200 mL of water. The mixture was stirred 5 minutes, allowed to settle and the layers were separated. The lower aqueous layer was re-extracted with EtOAc (2×250 mL). The organics were combined, washed with saturated NaCl, dried over Na₂SO₄, filtered and concentrated to an orange oil. Chromatography through a short silica gel plug eluted with EtOAc/Heptane (5-50%) afforded 15.5 g of product as an orange oil. ¹H NMR agrees with previously results for 2-hydroxymethyl-1-p-tolyl-cyclopropancarbonitrile. The ¹H NMR indicated about a 2.6:1 ratio of cis to trans isomers. Z-diastereomer ¹H NMR CDCl₃ (400 MHz) δ 1.57 (m, 2H, ArCCH₂CH), 1.87 (m, 1H, ArCCH₂CH), 2.11 (m, 1H, ArCCH₂CH), 2.33 (s, 3H, CH₃), 3.77 (m, 1H), 4.00 (m, 1H), 7.19 (m, 4H, ArH); E-diastereomer ¹H NMR CDCl₃ (400 MHz) δ 1.48 (dd, 1H, ArCCH₂CH, J=7.0 Hz, J=5.9 Hz), 1.69 (dd, 1H, ArCCH₂CH, J=9.4 Hz, J=5.9 Hz), 2.33 (s, 3H, CH₃), 3.14 (m, 1H), 3.40 (m, 1H,), 7.17 (m, 4H, ArH).

Method 2

A flask was charged with 1.5 liters of THF, 200.0 g (1524.4 mmol) of p-tolyl acetonitrile and 155.5 g (1680.5 mmol, 1.1 equiv) (S)-epichlorohydrin. The colorless mixture was cooled to −28° C. (inside temperature) under stirring. Then, 1.70 liters (3400.0 mmol, 2.23 equiv) of a 2 M THF solution of sodium bis-(trimethylsilyl)amide were added dropwise keeping the inside temperature between −28° C. and −20° C. The mixture turns to a yellowish then orange color (time of addition: ca. 15 min). After full addition the mixture was stirred another 4 h at −25° C. (±2° C.) for full conversion (HPLC-control). Then, the mixture was cooled to −60° C. At this temperature 1.75 liters (7012.2 mmol, 4.6 equiv) of a 4 M solution of HCl in dioxane were added within 120 min keeping the inside temperature between −60° C. and −55° C. The mixture was then allowed to warm to room temperature under stirring.

The mixture was taken in tert-butyl methyl ether (2 L) and water (1.8 L). The water layer was separated and the organic layer washed with water (2×1.5 L). The combined aqueous layer was reexttracted with in tert-butyl methyl ether (1.2 L). The combined organic layer was dried over sodium sulphate, filtered and concentrated on rotavap (20 mbar) then in high vacuum to afford 300.6 g (105% crude yield) of an orange crude oil.

The NMR and HPLC spectra of the crude material show a ca. 6.2:1 ratio of Z to E-isomer. The HPLC purity of Z+E was ca. 95 area % @220 nm.

### B. Synthesis of ((1R,2S)-2-(Aminomethyl)-2-p-tolylcyclopropyl)methanol

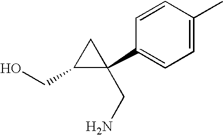

Method 1

To a stirring slurry of LAH (6.3 g, 0.165 moles) in diethyl ether (250 mL) at ambient temperature under nitrogen was added a solution of crude nitrile (15.5 g, (0.083 moles) in 75 mL of Et₂O), slowly via addition funnel while keeping the temperature below 30° C. The mixture was stirred at room temperature for 1-1.5 h after which time, no starting material was observed by TLC analysis (SiO₂ plate, EtOAc/Heptane 1:1). The reaction was allowed to stir for an additional 0.5 h then carefully quenched by the dropwise addition of H₂O (6.4 mL) followed by 7 mL of 25% NaOH and lastly 21 mL of H₂O. The resulting off white slurry was stirred for 1 h then filtered through a Celite pad, washing with Et₂O (3×250 mL). The filtrate was concentrated to give 15.7 g of crude product as a pale yellow oil. The product was carried forward crude without further purification. Crude ¹H NMR CDCl₃ (400 MHz, partial assignment), δ 0.71 (m, 1H), 0.92 (dd, 1H), 1.72 (m, 1H), 2.32 (s, 3H, CH₃), 2.54-2.57 (d, 1H, J=12.4 Hz), 3.33 (m, 1H), 4.09-4.13 (dd, 1H, J=12.3 Hz, J=1 Hz), 7.12 (m, 2H), 7.28 (m, 2H).

Method 2

An autoclave was charged with 3.0 L methanol, 450 g ammonia (liquid), 298.3 g (1511.9 mmol) of the crude product (described in section A, method 2 in this Example III) and 150 g of RaCo SK03/06 (prewashed with methanol). The autoclave was closed and purged three times with nitrogen (10 bar), then three times with hydrogen (10 bar). The heating was switched on and when the temperature reached 80° C. the pressure was set to 50 bar (725 psi) and stirring was started. After 20 h, the autoclave was cooled to room temperature and the pressure was released.

The mixture was filtered over a short pad of Hyflo and the filtrate was concentrated under reduced pressure on rotavap (20 mbar) then in high vacuum to obtain 311.0 g of an brownish crude oil. This crude material was taken in 1.2 liters of 2 M aqueous HCl. The aqueous layer was washed twice with dichloromethane (1×500 ml, 1×200 ml). The combined organic layer was reextracted with 0.2 M aqueous HCl (300 ml). The dichloromethane layer from the washing was put aside. The combined aqueous layer was basified with aqueous 25% ammonia to pH=8 and reextracted with dichloromethane (1×1 L, 2×250 ml). The combined organic layer was dried over sodium sulphate and concentrated under reduced pressure on rotavap (20 mbar) then in high vacuum to afford 232.0 g of a light-brown oil.

The material was dissolved in 300 ml acetonitrile. 250 ml HCl (6 M in 2-propanol) was added followed by ca. 1.1 l of diethyl ether. Previously prepared crystal germs of pure Z-isomer prodcut (HCl salt) were added to the cloudy mixture. After a first crystallization more material was obtained by adding another 200 ml of diethyl ether. The crystals were filtered off and washed with 2×120 ml diethyl ether/acetonitrile (1:1) and diethyl ether (2×100 ml) and dried in high vacuum (70° C., 2 h) to afford 152.3 g (44% yield) of the titlecompound as HCl salt as white crystals. The NMR and HPLC spectra of the crystals showed a ca. 97.3% chemical purity of desired Z-isomer. Ca. 1.7% of E-isomer impurity was present in the crystals.

Free base ¹H NMR (CDCl₃, 300 MHz) δ 7.32-7.12 (m, 4H, ArH), 4.12 (dd, J=12.2, 5.5 Hz, 1H), 3.46-3.30 (m, 2H), 2.90 (bs, 3H), 2.58 (d, J=12.2 Hz, 1H), 2.33 (s, 3H), 1.72 (m, 1H, ArCCH₂CH), 0.94 (dd, J=8.6, 4.7 Hz, 1H, ArCCH₂CH), 0.72 (m, 1H, ArCCH₂CH). HCl salt ¹H NMR (D6-DMSO, 300 MHz) δ 7.88 (bs, 3H, NH₃Cl), 7.29-7.14 (m, 4H, ArH), 5.25 (bs, 1H, OH), 3.87 (dd, J=12.0, 5.4 Hz, 1H, CH₂OH), 3.42-3.12 (m, 3H, CH₂OH, CH₂N), 2.28 (s, 3H), 1.37 (m, 1H, ArCCH₂CH), 1.04 (m, 1H, ArCCH₂CH), 0.94 (m, 1H, ArCCH₂CH).

### C. Synthesis of (1S,5R)-(−)-1-p-Tolyl-3-azabicyclo[3.1.0]hexane Hydrochloride

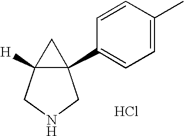

Method 1

To a stirring solution of crude amino alcohol (15.5 g, −0.081 moles) in 200 mL of DCE, at room temperature under nitrogen, was added 7.1 mL (0.097 moles, 1.2 eq) of SOCl₂ slowly via syringe while keeping the temperature below 45° C. (Note: The reaction exotherms from 22° C. to 42° C.) The resulting mixture was stirred for 3.5 h at ambient temperature after which time, TLC analysis (SiO₂ plate, CH₂Cl₂/MeOH/NH₄OH (10:1:0.1)) showed no starting material. The mixture was quenched with 75 mL of water and the layers were separated. The organic layer was washed with 2×150 mL of H₂O. The aqueous layers were combined, basified with 10N NaOH to pH=10 (pH paper) and the cloudy mixture was extracted with CH₂Cl₂ with (3×100 mL). The combined organics were dried over Na₂SO₄, filtered and concentrated to a dark oil. The oil was dissolved in MeOH (40 mL), treated with 40 mL of 2M HCl/Et₂O. The mixture was concentrated to approximately one eighth the original volume, diluted with CH₃CN (75 mL) heated to a gentle reflux (75-80° C.) for 1 minute then allowed to cool to room temperature and stand for 2 h. The resulting slurry was cooled to 10° C. and filtered followed by washing with 2×50 mL of CH₃CN. The product was dried 6 h (29 mmHg, 50° C.) to give 6.1 g (15% from p-tolylacetonitrile) of pure product as a white solid. mp=205-207° C.; ¹H NMR CDCl₃ (400 MHz) δ 1.20 (m, 1H, ArCCH₂CH), 1.51 (m, 1H, ArCCH₂CH), 1.92 (m, 1H, ArCCH₂CH), 2.32 (s, 3H, CH₃), 3.57 (m, 3H, CH₂N, CHN), 3.76 (m, 1H, CHN), 7.08 (m, 4H, ArH), 9.74 (br. s, 1H, NH), 10.24 (br.s, 1H, NHCl); ¹³C NMR (CDCl₃) δ 15.4, 21.2, 23.2, 31.2, 47.9, 51.1, 127.4, 129.7, 135.2, 137.3 (C, Ar); LC/MS (m/z M⁺ 188.1); Reverse Phase HPLC retention time=5.48 min; Normal Phase Chiral HPLC retention time=5.58 min; [α]²⁰_{D} 50 (c=1, MeOH).

Method 2

A flask was charged with 2.0 L ethyl acetate. 150.1 g (659.2 mmol) of the HCl salt prouct (described in section B, method 2 in this Example III) were added and the stirred white suspension was externally cooled with an ice/2-propanol-bath. 52.8 ml (725.1 mmol, 1.1 equiv) of thionyl chloride were added dropwise at 0° C. within 20 min. A small exotherm could be detected (the inside temperature rose from 0 to 3° C.). After full addition the mixture was stirred 1.5 h at low temperature (0-3° C.). The initial suspension turned almost completely homogeneous. Then 824 ml (5526.0 mmol, 8.0 equiv) of 12.5% ammonium hydroxide were added within 60 min keeping the inside temperature below 5° C. with external cooling. The now white emulsion was allowed to warm to room temperature and stirred overnight. The aqueous layer was separated and reextracted with 500 ml ethyl acetate. The combined ethyl acetate layer was dried over sodium sulphate and concentrated on rotavap (20 mbar) then in high vacuum to afford 121.0 g (106% crude yield) of a clear yellowish oil.

The crude material was dissolved in 600 ml ethyl acetate. To the filtrate was added 1.0 equiv of HCl/2-propanol under stirring at room temperature. Bicifadine HCl crystallized immediately from the mixture with low exotherms. The white crystals were filtered to afford 119.7 g (87% yield) of (−)-Bicifadine HCl. The NMR and HPLC spectra of the crystals show a >98% chemical purity. The material has >98% ee as measured by chiral HPLC. [α]²⁰_{D}=−60 (c=1, MeOH). ¹H NMR (D₆-DMSO, 300 MHz) δ 10.0 (bs, 2H, NH₂Cl), 7.15-7.07 (m, 4H, ArH), 3.78-3.56 (m, 4H, CH₂NCH₂), 2.33 (s, 3H), 1.92 (m, 1H, ArCCH₂CH), 1.54 (dd, J=6.6, 4.8 Hz, 1H. ArCCH₂CH), 1.20 (m, 1H, ArCCH₂CH).

### EXAMPLE V

### Preparation of (1S,5R)-1-(4-Methoxyphenyl)-3-aza-bicyclo[3.1.0]hexane Using Reaction Scheme 6

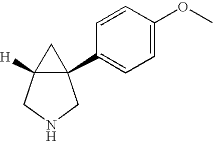

4-Methoxyphenylacetonitrile (12 g, 0.085 moles) was dissolved in tetrahydrofuran (100 ml) and cooled in an ice bath to 0-5° C. Sodium bis (trimethylsilyl)amide (170 ml, 0.085 moles) was added dropwise at such a rate that the temperature remained below 10° C. The addition occurred over twenty minutes. The mixture was stirred for an additional 30 minutes at 0-5° C. The (R)-(−)-epichlorohydrin in tetrahydrofuran (10 ml) was added over twenty minutes. The addition funnel was rinsed with tetrahydrofuran (10 ml). A second equivalent of sodium bis(trimethylsilyl)amide (170 ml, 0.085 moles) was added dropwise over twenty minutes at 0-5° C. After stirring at 0-5° C. for twenty minutes the reaction was quenched with water (100 ml) and extracted with ethyl acetate (2×100 ml). The combined organic portions were washed with brine (100 ml) and dried over magnesium sulfate, filtered and concentrated under reduced pressure to yield an orange oil (30.9 g). The oil was purified by silica gel chromatography (10% ethyl acetate: heptane (2 L), 20% ethyl acetate: heptane (1 L), 30% ethyl acetate: heptane (1 L) and finally 40% ethyl acetate: heptane (1 L). Isolated 2-hydroxymethyl-1-(4-methoxyphenyl)-cyclopropanecarbonitrile (7.17 g, 41% yield) was obtained as a clear yellow oil. ¹H NMR (CDCl₃) (partial assignment) δ ppm 7.36 (1H, d), 7.27 (2H, d), 6.90 (2H, d), 4.07 (1H, m), 3.81 (3H, s).

Lithium aluminum hydride (2.68 g 0.0705 moles) was suspended in diethyl ether (125 ml). 2-hydroxymethyl-1-(4-methoxyphenyl)-cyclopropanecarbonitrile (7.17 g, 0.353 moles) in diethyl ether (30 ml) was added dropwise over 45 minutes. After an additional 45 minutes TLC (1:1; ethyl acetate: heptane and 20:1:0.1; dichloromethane: methanol: ammonium hydroxide) showed all the starting material had reacted. The reaction was quenched with water (2.9 ml), then sodium hydroxide (3M) (2.9 ml) and finally water (9 ml) and allowed to stir overnight. The reaction mixture was filtered through celite and rinsed with diethyl ether (100 ml). The diethyl ether was concentrated under reduced pressure to give [2-aminomethyl-2-(4-methoxyphenyl)-cyclopropyl]-methanol (6.8 g, 93% yield) as an orange oil which was used without further purification.

[2-Aminomethyl-2-(4-methoxyphenyl)-cyclopropyl]-methanol (6.80 g, 0.0328 moles) was dissolved in dichloroethane (55 ml). Thionyl chloride (5.07 g, 0.0426 moles) was added dropwise via syringe. After 3 hours the reaction was complete as shown by HPLC and quenched with water (100 ml). The mixture was extracted with dichloromethane (100 ml). The organic portion was washed with water (100 ml). The combined aqueous portions were basified with sodium hydroxide (10 N) to pH=10 and then extracted with dichloromethane (2×100 ml). The organic portion was dried over magnesium sulfate, filtered and concentrated under reduced pressure to a clear oil. Methanol (20 ml) was added to the oil and 2 M HCl/ether (16 ml). Most of the methanol was removed under reduced pressure and acetonitrile (25 ml) was added. A white solid precipitated from the clear green solution. The solution was cooled to 0-5° C. in an ice bath and filtered. Concentrating the mother liquor to dryness and treating the residue with acetonitrile, cooling to 0-5° C. and filtering, gave a second crop. The HPLC chiral purity was >99% ee for each crop so they were combined and dried in vacuo at 50° C. for 12 hours. The white solid was identified as (1S, 5R)-1-(4-methoxyphenyl)-3-aza-bicyclo[3.1.0]hexane hydrochloride (1.75 g, 24% yield). ¹H NMR (CDCl₃) δ ppm 10.29 (1H, bs), 9.75 (1H, bs), 7.15 (2H, d), 6.87 (2H, d), 3.81 (3H, s), 3.6 (4H, m), 1.90 (1H, m), 1.48 (1H, t), 1.21 (1H, t). MS (M+1) 190.

### EXAMPLE VI

### Preparation of (1R,5S)-1-(4-Methoxyphenyl)-3-aza-bicyclo[3.1.0]hexane Using Reaction Scheme 5

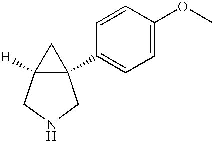

The indicated compound was prepared using the the same procedure used to make (1S, 5R)-1-(4-methoxyphenyl)-3-aza-bicyclo[3.1.0]hexane hydrochloride (see Example V hereinabove), except that the (S)-(+)-epichlorohydrin was used instead of (R)-(−)-epichlorohydrin. Isolated 2-hydroxymethyl-1-(4-methoxyphenyl)-cyclopropanecarbonitrile (5.00 g, 27% yield) was obtained as a clear yellow oil. ¹H NMR (CDCl₃) (partial assignment) δ ppm 7.36 (1H, d), 7.27 (2H, d), 6.90 (2H, d), 4.07 (1H, m), 3.81 (3H, s).

Isolated [2-aminomethyl-2-(4-methoxyphenyl)-cyclopropyl]-methanol (4.59 g, 90% yield) was obtained as an yellow oil and was used without further purification.

The HPLC chiral purity was >99% ee for the first crop and 94% ee for the second. The second crop was recrystallized from a minimum amount of hot methanol. Chiral purity for the recrystallized material by HPLC was now 99.3% ee. The two crops were combined and dried in vacuo at 50° C. for 12 hours. The white solid was identified as (1R,5S)-1-(4-methoxyphenyl)-3-aza-bicyclo[3.1.0]hexane hydrochloride (0.95 g, 19% yield). ¹H NMR (CDCl₃) δ ppm 10.29 (1H, bs), 9.75 (1H, bs), 7.15 (2H, d), 6.87 (2H, d), 3.81 (3H, s), 3.6 (4H, m), 1.90 (1H, m), 1.48 (1H, t), 1.21 (1H,t). MS (M+1) 190.

### EXAMPLE VII

### Preparation of 3-Substituted 1-Aryl-3-azabicyclo[3.1.0]hexanes Using Reaction Scheme 11

### A. General Synthetic Procedure for Alkylation of 1-aryl-3-azabicyclo[3,1,0]hexanes

Pursuant to reaction Scheme 11, to a stirred solution of a 1-aryl-3-azabicyclo[3,1,0]hexane (1 eq) in anhydrous DMF (15 mL) was added diisopropylethylamine (DIPEA) (1.3 eq). The reaction mixture was stirred at room temperature for 20 minutes then alkyl halides (1.3 eq) were added to the reaction mixture and then allowed to stir at room temperature for 2 hours and analyzed by TLC. If unreacted starting material remained, the reactions were warmed to 50° C. and held overnight. Reactions were reduced under a high vacuum then dissolved in dichloromethane (20 mL) and washed with water (20 mL). The reaction mixture was passed through a phase separator cartridge. Organics were collected and filtered through a 2 g silica cartridge, fractions were monitored by TLC, the fractions contained the desired product were combined, reduced and analysed by ¹H-NMR. The free bases of the compounds shown below (NMR data also listed below) in Section C of this Example VII were prepared using the general procedure described above.

### B. General Procedure for Hydrochloride Salt Formation

To a stirred solution of free base (I mol equiv.) in anhydrous diethyl ether (5 mL) was added 1M HCl in ether (5 mol equiv.) dropwise. On complete addition the reaction mixture was stirred at ice bath temperature for 30 minutes. The resultant solids were isolated by filtration, washing with cold diethyl ether (5 mL). The isolated solids were oven dried and analyzed by ¹H-NMR, ¹³C-NMR and MS. The hydrochloride salts of the compounds shown below (NMR data also listed below) in Section C of this Example VII were prepared using the general procedure described above.

### C. Representative Compounds Prepared

### (1) 3-Propyl-1-p-tolyl-3-aza-bicyclo[3.1.0]hexane

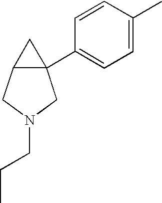

Free base, 0.9284 g (yield 60%): ¹H NMR (300 MHz, δ₆-DMSO) δ 7.11-7.04 (m, 4H, ArH), 3.34 (d, 1H, J=8.4 Hz, NCH₂), 3.12 (d, 1H, J=8.9 Hz, NCH₂), 2.55 (d, 1H, J=8.5 Hz, NCH₂), 2.44 (m, 3H, NCH₂, CH₂CH₂CH₃), 2.32 (s, 3H, ArCH₃), 1.66 (m, 1H, CH₂CH), 1.50 (m, 2H, CH₂CH₂CH₃), 1.39 (t, 1H, J=4.3 Hz, CHCH₂), 0.90 (t, 3H, J=7.4 Hz, CH₂CH₃), 0.77 (dd, 1H, J=7.7 Hz, 4.1 Hz, CHCH₂). Hydrochloride salt: ¹H NMR (300 MHz, δ₆-DMSO) δ 11.13 (s, 1H, NHCl), 7.34-7.14 (m, 4H, ArH), 3.90 (dd, 1H, J=11.1 Hz, 5.2 Hz, NCH₂), 3.63 (dd, 1H, J=11.0 Hz, 5.5 Hz, NCH₂), 3.52-3.39 (m, 2H, 2×NCH₂), 3.07 (m, 2H, NCH₂CH₂CH₃), 2.29 (s, 3H, ArCH₃), 2.08 (m, 1H, CHCH₂), 1.84 (m, 1H, CHCH₂), 1.76 (m, 2H, NCH₂CH₂CH₃), 1.01 (t, 1H, J=6.6 Hz, CHCH₂), 0.89 (t, 3H, J=7.3 Hz, NCH₂CH₂CH₃); ¹³C NMR (75 MHz, δ₆-DMSO) δ 136.9, 136.5, 129.7, 127.3, 57.9, 56.7, 55.5, 30.4, 23.4, 21.3, 19.1, 16.3, 11.7; MS (m/z) 216 (MH⁺, 100).

### (2) 3-Isopropyl-1-p-tolyl-3-aza-bicyclo[3.1.0]hexane

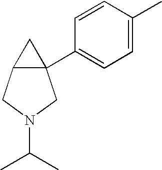

Free base, 0.6645 g (yield 43%): ¹H NMR (300 MHz, δ₆-DMSO) δ 7.76-7.05 (m, 4H, ArH), 3.38 (d, 1H, J=8.5 Hz, NCH₂), 3.15 (d, 1H, J=8.8 Hz), 2.62 (d, 1H, J=8.4 Hz, NCH₂), 2.52 (dd, 1H, J=8.8 Hz, 3.7 Hz, NCH₂), 2.47 (m, 1H, NCH₂), 2.32 (s, 3H, ArCH₃), 1.66 (m, 1H, CH₂CH), 1.37 (t, 1H, J=4.0 Hz, NCH₂), 1.07 (dd, 6H, J=3.7 Hz, 6.7 Hz, ((CH₃)₂CH), 0.76 (dd, 1H, J=8.1 Hz, 4.1 Hz, CHCH₂). Hydrochloride salt: ¹H NMR (300 MHz, δ₆-DMSO) δ 11.01 (s, 1H, NHCl), 7.21-7.14 (m, 4H, ArH), 3.91 (dd, 1H, J=11.0 Hz, 5.5 Hz, NCH₂), 3.61 (dd, 1H, J=11.0 Hz, 5.5 Hz, NCH₂), 3.54-3.34 (m, 3H, 2×NCH₂, NCH(CH₃)₂), 2.29 (s, 3H, ArCH₃), 2.10 (m, 1H, CHCH₂), 1.90 (t, 1H, J=5.5 Hz, CHCH₂), 1.36 (t, 6H, J=7.0 Hz, NCH(CH₃)₂), 0.98 (t, 1H, J=6.2 Hz, CHCH₂); C³C NMR (75 MHz, δ₆-DMSO) δ 136.5, 135.9, 129.1, 126.7, 58.3, 56.3, 53.6, 22.9, 20.8, 18.7, 18.6, 15.9; MS (m/z) 216 (MH⁺, 100).

### (3) 3-Isobutyl-1-p-tolyl-3-aza-bicyclo[3.1.0]hexane

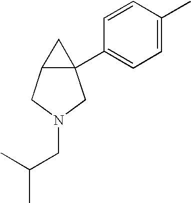

Free base, 0.8059 g (yield 49%): ¹H NMR (300 MHz, δ₆-DMSO) δ 7.25-7.05 (m, 4H, ArH), 3.30 (d, 1H, J=8.4 Hz, NCH₂), 3.08 (d, 1H, J=8.5 Hz, NCH₂), 2.51 (d, 1H, J=8.1 Hz, NCH₂), 2.45 (dd, 1H, J=8.4 Hz, 3.6 Hz, NCH₂), 2.34 (s, 3H, ArCH₃), 2.23 (d, 2H, J=7.0 Hz), NCH₂CH), 1.74 (m, 1H, CH₂CH(CH₃)₂), 1.65 (m, 1H, CH₂CH), 1.43 (t, 1H, J=4.1 Hz, CHCH₂), 0.89 (d, 6H, J=6.7 Hz, CH(CH₃)₂), 0.74 (dd, 1H, J=8.1 Hz, 3.7 Hz, CHCH₂). Hydrochloride salt: ¹H NMR (300 MHz, δ₆-DMSO) δ 10.67 (s, 1H, NHCl), 7.21-7.14 (m, 4H, ArH), 4.01 (dd, 1H, J=11.0 Hz, 5.5 Hz, NCH₂), 3.73 (dd, 1H, J=11.1 Hz, 5.6 Hz, NCH₂), 3.52 (m, 2H, 2×NCH₂), 3.05 (t, 2H, J=5.6 Hz, CH₂CH(CH₃)₂), 2.29 (s, 3H, ArCH₃), 2.08 (m, 2H, CH₂CH(CH₃)₂, CHCH₂), 2.00 (t, 1H, J=7.0 Hz, CHCH₂), 1.00 (d, 7H, J=3.3 Hz, NCH₂CH(CH₃)₂, CHCH₂); ¹³C NMR (75 MHz, δ₆-DMSO) δ=144.5, 144.1, 137.2, 134.9, 70.5, 66.5, 64.1, 38.2, 33.4, 31.1. 29.3, 28.9, 24.1; MS (m/z) 230 (MH⁺, 100).

### (4) 3-(2-Methoxyethyl)-1-p-tolyl-3-aza-bicyclo[3.1.0]hexane

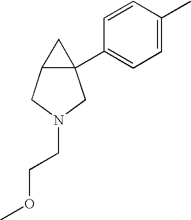

Free base, 0.092 g (yield 5%): ¹H NMR (300 MHz, δ₆-DMSO) δ 71.4-7.02 (m, 4H, ArH), 3.46 (t, 3H, J=5.7 Hz, NCH₂CH₂OCH₃), 3.34 (s, 3H, OCH₃), 3.12 (d, 1H, J=8.5 Hz, NCH₂), 2.67 (t, 2H, J=5.9 Hz, NCH₂CH₂)CH₃), 2.60 (d, 1H, J=8.4 Hz, NCH₂), 2.50 (dd, 1H, J=8.8 Hz, 5.1 Hz, NCH₂), 2.31 (s, 3H, ArCH₃), 1.63 (m, 1H, CH₂CH), 1.40 (t, 1H, J=4.1 Hz, CHCH₂), 0.76 (dd, 1H, J=8.0 Hz, 4.4 Hz, CHCH₂). Hydrochloride salt: ¹H NMR (300 MHz, δ₆-DMSO) δ 7.21-7.14 (m, 4H, ArH), 3.90 (dd, 1H, J=11.0 Hz, 5.2 Hz, NCH₂), 3.78 (m, 2H, NCH₂CH₂OCH₃), 3.67 (dd, 1H, J=11.0 Hz, 5.1 Hz, NCH₂), 3.54 (m, 2H, 2×NCH₂), 3.41 (m, 2H, NCH₂CH₂OCH₃), 3.31 (s, 3H, NCH₂CH₂OCH₃), 2.29 (s, 3H, ArCH₃), 2.09 (m, 1H, CHCH₂), 1.75 (t, 1H, J=5.9 Hz, CHCH₂), 1.02 (t, 1H, J=6.6 Hz, CHCH₂); ¹³C NMR (75 MHz, δ₆-DMSO) δ 144.4, 144.2, 137.2, 134.9, 75.2, 66.4, 66.4, 63.9, 61.8, 37.9, 30.9, 28.8, 23.6; MS (m/z) 232 (MH⁺, 100).

### (5) (1R,5S)-3-Methyl-1-p-tolyl-3-aza-bicyclo[3.1.0]hexane

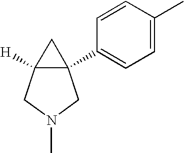

Hydrochloride salt, 0.59 g (yield 26%): ¹H NMR (300 MHz, d₆-DMSO) δ 11.21 (1H, brs, NH⁺), 7.17-7.11 (4H, m, ArH), 3.84 (1H, dd, J=11.1, 5.4 Hz, HCH), 3.58 (1H, dd, J=1.1,5.1 Hz, HCH), 3.50-3.40 (2H, m, CH₂), 2.78 (3H, d, J=4.8 Hz, NCH₃), 2.26 (3H, s, ArCH₃), 2.07-2.02 (1H, m, CH), 1.75 (1H, t, J=5.7 Hz, HCH), 1.00 (1H, t, J=6.9 Hz). ¹³C NMR (75 MHz, d₆-DMSO) δ 135.96, 135.71, 128.88, 126.44, 58.51, 55.95, 29.90, 22.95, 20.47, 15.13. MS (m/z) 188 (MH⁺, 100). Chiral purity >97% ee.

### (6) (1S,5R)-3-Methyl-1-p-tolyl-3-aza-bicyclo[3.1.0]hexane

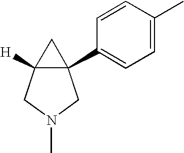

Hydrochloride salt, 0.51 g (yield 28%): ¹H NMR (300 MHz, d₆-DMSO) δ 11.00 (1H, brs, NH⁺), 7.17-7.11 (4H, m, ArH), 3.85 (1H, dd, J=11.1, 5.7 Hz, HCH), 3.60 (1H, dd, J=11.1, 5.1 Hz, HCH), 3.51-3.40 (2H, m, CH₂), 2.79 (3H, d, J=4.5 Hz, NCH₃), 2.26 (3H, s, ArCH₃), 2.08-2.02 (1H, m, CH), 1.68 (1H, t, J=6.0 Hz, HCH), 1.02 (1H, t, J=6.9 Hz, HCH). ¹³C NMR (75 MHz, d₆-DMSO) δ 135.93, 135.74, 128.89, 126.45, 58.59, 56.04, 29.90; 22.94, 20.48, 15.13. MS (m/z) 188 (MH⁺, 100). Chiral purity >97% ee.

### (7) (1R,5S)-3-Ethyl-1-p-tolyl-3-aza-bicyclo[3.1.0]hexane

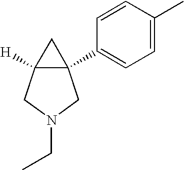

Hydrochloride salt, 0.49 g (yield 21%): ¹H NMR (300 MHz, d₆-DMSO) δ 10.96 (1H, brs, NH⁺), 7.17-7.10 (4H, m, ArH), 3.88 (1H, dd, J=11.1, 5.1 Hz, HCH), 3.60 (1H, dd, J=11.1, 5.1 Hz, HCH), 3.47-3.37 (2H, m, NCH₂CH₃), 3.21-3.05 (2H, m, CH₂), 2.26 (3H, s, ArCH₃), 2.09-2.03 (1H, m, CH), 1.75 (1H, t, J=5.4 Hz, HCH), 1.26 (3H, t, J=7.2 Hz, NCH₂CH₃), 0.99 (1H, t, J=6.9 Hz, HCH). ¹³C NMR (75 MHz, d₆-DMSO) δ 136.07, 135.68, 128.86, 126.41, 56.69, 54.23, 49.45, 29.50, 22.49, 20.47, 15.50, 10.40. MS (m/z) 202 (MH⁺, 100). Chiral purity >97% ee.

### (8) (1S,5R)-3-Ethyl-1-p-tolyl-3-aza-bicyclo[3.1.0]hexane

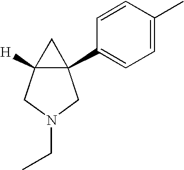

Hydrochloride salt, 0.73 g (yield 44%): ¹H NMR (300 MHz, d₆-DMSO) δ 10.89 (1H, brs, NH⁺), 7.21-7.15 (4H, m, ArH), 3.93 (1H, dd, J=11.1, 5.1 Hz, HCH), 3.64 (1H, dd, J=11.1, 5.1 Hz, HCH), 3.51-3.42 (2H, m, NCH₂CH₃), 3.26-3.17 (2H, m, CH₂), 2.30 (3H, s, ArCH₃), 2.13-2.08 (1H, m, CH), 1.75 (1H, t, J=6.0 Hz, HCH), 1.29 (3H, t, J=7.5 Hz, NCH₂CH₃), 1.03 (1H, t, J=6.6 Hz, HCH). ¹³C NMR (75 MHz, d₆-DMSO) δ 136.05, 135.68, 128.86, 126.40, 56.72, 54.27, 49.46, 29.49, 22.49, 20.47, 15.50, 10.43. MS (m/z) 202 (MH⁺, 100). Chiral purity >97% ee.

### (9) (1R,5S)-3-Isopropyl-1-p-tolyl-3-aza-bicyclo[3.1.0]hexane

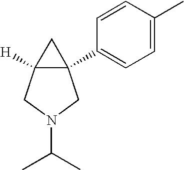

Hydrochloride salt, 1.273 g (41%): ¹H NMR (300 MHz, d₆-DMSO) δ 10.94 (1H, brs, NH⁺), 7.18-7.11 (4H, m, ArH), 3.90 (1H, dd, J=11.1, 5.4 Hz, NCH₂), 3.60 (1H, dd, J=11.1, 5.1 Hz, NCH₂), 3.50-3.39 (3H, m, NCH₂, NCH), 2.26 (3H, s, ArCH₃), 2.10-2.05 (1H, m, CH), 1.88 (1H, obs t, J=5.1 Hz, HCH), 1.33 (6H, obs t, J=7.2 Hz, CH₃), 0.97 (1H, obs t, J=7.2 Hz, HCH); ¹³C NMR (75 MHz, d₆-DMSO) δ 136.2, 135.6, 128.9, 126.4, 58.0, 56.0, 53.3, 29.7, 22.7, 20.5, 18.4, 15.7; MS (m/z) 216 (MH⁺, 100).

### (10) (1S,5R)-3-Isopropyl-1-p-tolyl-3-aza-bicyclo[3.1.0]hexane

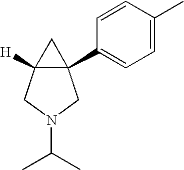

Hydrochloride salt, 1.179 g (38%): ¹H NMR (300 MHz, d₆-DMSO) δ 11.01 (1H, brs, NH⁺), 7.20-7.10 (4H, m, ArH), 3.90 (1H, dd, J=11.1, 5.4 Hz, NCH₂), 3.82 (1H, dd, J=11.1, 5.7 Hz, NCH₂), 3.50-3.39 (3H, m, NCH₂, NCH), 2.26 (3H, s, ArCH₃), 2.10-2.05 (1H, m, CH), 1.88 (1H, obs t, J=5.1 Hz, HCH), 1.33 (6H, obs t, J=7.2 Hz, CH₃), 0.97 (1H, obs t, J=7.2 Hz, HCH); ¹³C NMR (75 MHz, d₆-DMSO) δ 136.2, 135.6, 128.8, 126.4, 58.0, 56.0, 53.3, 29.7, 22.7, 20.5, 18.4, 15.7; MS (m/z) 216 (MH⁺, 100).

### (11) 3-Methyl-1-p-tolyl-3-aza-bicyclo[3.1.0]hexane

Free base, 0.6871 g (yield: 51%): ¹H NMR (300 MHz, δ₆-DMSO) δ 7.10-7.03 (m, 4H, ArH), 3.28 (d, 1H, J=8.5 Hz, NCH₂), 3.07 (d, 1H, J=8.8 Hz, NCH₂), 2.55 (d, 1H, J=8.4 Hz, NCH₂), 2.47 (dd, 1H, J=8.8 Hz, 5.1 Hz, NCH₂), 2.37 (s, 3H, NCH₃), 2.30 (s, 3H, ArCH₃), 1.65 (m, 1H, CH₂CH), 1.38 (t, 1H, J=4.0 Hz, CHCH₂), 0.77 (dd, 1H, J=8.1 Hz, 4.4 Hz, CHCH₂). Hydrochloride salt: ¹H NMR (300 MHz, δ₆-DMSO) δ 11.36 (s, 1H, NHCl), 7.20-7.12 (m, 4H, ArH), 3.86 (dd, 1H, J=11.0 Hz, 5.1 Hz, NCH₂), 3.60 (dd, 1H, J=11.1 Hz, 5.2 Hz, NCH₂), 3.53-3.43 (m, 2H, 2×NCH₂), 2.80 (s, 3H, NCH₃), 2.28 (s, 3H, ArCH₃), 2.07 (m, 1H, CHCH₂), 1.81 (t, 1H, J=5.2 Hz, CHCH₂), 1.02 (t, 1H, J=7.4 Hz, CHCH₂); ¹³C NMR (75 MHz, δ₆-DMSO) δ 136.0, 135.7, 128.9, 126.5, 58.5, 55.9, 29.9, 23.0, 20.5, 15.2; MS (m/z) 188 (MH⁺, 100).

### (12) 3-Ethyl-1-p-tolyl-3-aza-bicyclo[3.1.0]hexane

Free base, 1.0324 g (yield: 72%): ¹H NMR (300 MHz, δ₆-DMSO) δ 7.11-7.04 (m, 4H, ArH), 3.35 (d, 1H, J=8.4 Hz, NCH₂), 3.12 (d, 1H, J=8.5 Hz, NCH₂), 2.56-2.43 (m, 4H, 2×NCH₂, CH₃CH₂), 2.32 (s, 3H, NCH₃), 1.66 (m, 1H, CH₂CH), 1.39 (t, 1H, J=4.4 Hz, CHCH₂), 1.09 (t, 3H, J=7.4 Hz, CH₂CH₃), 0.78 (dd, 1H, J=7.7 Hz, 4.0 Hz, CHCH₂). Hydrochloride salt: ¹H NMR (300 MHz, δ₆-DMSO) δ 1.06 (s, 1H, NHCl), 3.92 (dd, 1H, J=11.0 Hz, 5.1 Hz, NCH₃), 3.64 (dd, 1H, J=11.0 Hz, 5.5 Hz, NCH₂), 3.50-3.39 (m, 2H, 2×NCH₂), 3.20 (m, 2H, NCH₂CH₃), 2.29 (s, 3H, ArCH₃), 2.09 (m, 1H, CHCH₂), 1.81 (m, 1H, CHCH₂), 1.29 (t, 3H, J=7.4 Hz, NCH₂CH₃), 1.02 (t, 1H, J=6.6 Hz, CHCH₂); ¹³C NMR (75 MHz, δ₆-DMSO) δ=136.1, 135.7, 128.9, 126.4, 56.7, 54.2, 49.4, 29.5, 22.5, 20.5, 15.5, 10.4; MS (m/z) 202 (MH⁺, 100).

### (13) 1-p-Tolyl-3-trifluoromethyl-3-aza-bicyclo[3.1.0]hexane

Free base, 0.6050 g (yield: 53%): ¹H NMR (300 MHz, δ₆-DMSO) δ 7.16-7.06 (m, 4H, ArH), 3.97 (t, 1H, J=6.3 Hz, NCH₂), 3.78 (s, 3H, NCH₂), 2.34 (s, 3H, ArCH₃), 1.87 (m, 1H, CHCH₂), 1.19 (t, 1H, J=5.5 Hz, CHCH₂), 0.87 (m, 1H, CHCH₂). Hydrochloride salt: ¹H NMR (300 MHz, δ₆-DMSO) δ 7.14 (s, 4H, ArH), 3.94-3.49 (m, 4H, 4×NCH₂), 2.28 (s, 3H, ArCH₃), 2.01 (m, 1H, CHCH₂), 1.09 (t, 1H, J=5.2 Hz, CHCH₂), 0.89 (t, 1H, J=4.8 Hz, CHCH₂); ¹³C NMR (75 MHz, δ₆-DMSO) δ 155.5, 151.7, 145.4, 143.6, 137.2, 134.7, 60.8, 60.3, 57.7, 57.2, 38.8, 38.2, 31.8, 31.3, 28.8, 26.4, 26.3; MS (m/z) 242 (MH⁺, 5).

### EXAMPLE VIII

### Preparation of 1-p-Tolyl-3-(2,2,2-trifluoroethyl)-3-aza-bicyclo[3.1.01 hexane Using Reaction Scheme 11

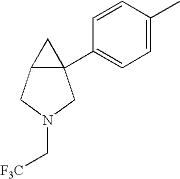

A solution of bicifadine (2 g, 9.54 mmol) and triethylamine (1.33 mL, 9.54 mmol) and 2,2,2-trifluoroethyltrichloromethane sulphonate (0.7 mL, 4.4 mmol) in toluene (20 mL) was heated to reflux and held at this temperature until complete conversion by TLC was observed. The reaction mixture was partitioned between ethyl acetate (50 mL) and saturated sodium bicarbonate solution (50 mL). Organics were isolated, dried over magnesium sulphate, filtered and reduced. Crude material was purified by column chromatography [SiO₂ (30 g): (90 EtOAc: 8 MeOH: 2 NH₄OH)] to give the required material as a yellow oil, 0.9149 g (75%): ¹H NMR (300 MHz, δ₆-DMSO) δ 7.26-7.05 (m, 4H, ArH), 3.44 (d, 1H, J=8.1 Hz, NCH₂), 3.23-3.08 (m, 3H, CH₂CF₃, NCH₂), 2.90 (d, 1H, J=8.1 Hz, NCH₂), 2.84 (dd, 1H, J=8.1 Hz, 4.1 Hz, NCH₂), 2.37 (s, 3H, ArCH₃), 1.71 (m, 1H, CH₂CH), 1.38 (t, 1H, J=4.4 Hz, CHCH₂), 0.83 (dd, 1H, J=7.7 Hz, 4.0 Hz, CHCH₂). Hydrochloride salt: ¹H NMR (300 MHz, δ₆-DMSO) δ 7.18-7.12 (m, 4H, ArH), 4.01 (m, 2H, 2×NCH₂), 3.75 (m, 1H, NCH₂), 2.51 (m, 3H, NCH₂CF₃, NCH₂), 2.28 (s, 3H, ArCH₃), 2.00 (m, 1H, CHCH₂), 1.70 (m, 1H, CHCH₂), 0.96 (m, 1H, CHCH₂); ¹³C NMR (75 MHz, 66-DMSO) δ 145.32, 143.79, 137.21, 134.82, 67.26, 64.41, 61.95, 61.56, 61.13, 60.71, 38.61, 31.70, 28.85; MS (m/z) 256 (MH⁺, 100).

### EXAMPLE IX

### Preparation of 1-Aryl-3-methyl-3-aza-bicyclo[3.1.0]hexane hydrochlorides Using Reaction Scheme 14

### A. Synthesis of 3-Bromo-1-methyl-1H-pyrrole-2,5-dione

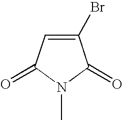

Pursuant to steps a and b of Reaction Scheme 14, a solution of bromomaleic anhydride (52.8 g, 0.298 mol) in diethyl ether (250 mL) was cooled to 5° C. A 2 M solution of methylamine in THF (298 mL, 0.596 mol, 2 eq.) was added dropwise over 1 hour and the reaction stirred for a further 30 minutes, maintaining the temperature below 10° C. The resulting precipitate was filtered, washed with diethyl ether (2×100 mL) and air-dried for 30 minutes then suspended in acetic anhydride (368 mL) and sodium acetate (12.2 g, 0.149 mol, 0.5 eq.) added. The reaction was heated to 60° C. for 2 hours and then solvent was removed in vacuo. The residue was taken up in DCM (500 mL) and washed with saturated sodium bicarbonate solution (2×500 mL) and water (2×300 mL). Organics were dried over MgSO₄ (89 g), filtered and reduced in vacuo. The resulting oil was azeotroped with toluene (4×100 mL) to give N-methyl bromomaleimide as a beige solid. Yield=41.4 g (73%); ¹H NMR (300 MHz, CDCl₃) δ 6.95 (1H, s, CH), 3.07 (3H, s, NCH₃)

### B. General Synthetic Procedure for Preparation of 3-(Aryl)-1-methyl-pyrrole-2,5-diones

Pursuant to step c of Reaction Scheme 14, the following provides a general procedure for synthesis of 3-aryl-1-methyl-pyrrole-2,5-diones. N-Methyl bromomaleimide (20 mL of a 0.5 M solution in 1,4-dioxane, 1.96 g net, 10 mmol), aryl boronic acid (11 mmol, 1.1 eq.), cesium fluoride (3.4 g, 22 mmol, 2.2 eq.) and [1,1′-bis-(diphenylphosphino)ferrocene]palladium (II) chloride (0.4 g, 0.5 mmol, 5 mol %) were stirred at 40° C. for between 1 and 6 hours. Reactions were filtered, solids washed with 1,4-dioxane (5 mL) and solvents removed in vacuo (two of the solids required an extra wash with dichloromethane at this stage). Residues were taken up in DCM (5 mL) then purified either by passing through a flash silica chromatography cartridge (20 g silica) or by column chromatography (30 g silica, eluted with 4:1 hexane:ethyl acetate then 2:1 hexane:ethyl acetate). Solvents were removed in vacuo to give the required crude products as solids. The compounds shown below (NMR data also listed below) were prepared using the foregoing general procedure:

### (1) 1-Methyl-3-(4-trifluoromethyl)phenyl)pyrrole-2,5-dione

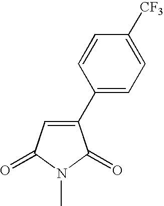

Yield=1.4 g (53%); ¹H NMR (300 MHz, CDCl₃) δ 8.04-8.01 (2H, d, J=8.5 Hz, ArH), 7.74-7.67 (2H, m, ArH), 6.84 (1H, s, CH), 3.09 (3H, s, NCH₃); MS (m/z) 256 [MH⁺].

### (2) 3-(3-Chlorophenyl)-1-methyl-pyrrole-2,5-dione

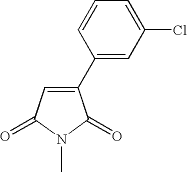

Yield=3.7 g (83%); ¹H NMR (300 MHz, CDCl₃) δ 7.91-7.90 (1H, t, J=1.8 Hz, ArH), 7.82-7.79 (1H, dt, J=7.3 Hz, 1.8 Hz ArH), 7.46-7.36 (2H, m, ArH), 6.75 (1H, s, CH), 3.08 (3H, s, NCH₃); MS (m/z) 222 [MH⁺].

### (3) 3-(4-Fluorophenyl)-1-methyl-pyrrole-2,5-dione

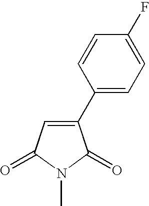

Yield=1.9 g (90%); ¹H NMR (300 MHz, CDCl₃) δ 7.97-7.92 (2H, m, ArH), 7.17-7.10 (2H, m, ArH), 6.68 (1H, s, CH), 3.07 (3H, s, NCH₃); MS (m/z) 206 [MH⁺].

### (4) 3-(1-Methyl-2,5-dioxo-2,5-dihydro-1H-pyrrol-3-yl)-benzonitrile

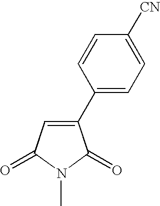

Yield=0.2 g (9%); ¹H NMR (300 MHz, CDCl₃) δ 8.05-8.02 (2H, d, J=8.5 Hz, ArH), 7.76-7.73 (2H, d, J=8.5 Hz, ArH), 6.86 (1H, s, CH), 3.09 (3H, s, NCH₃); MS (m/z) 213 [MH⁺].

### C. General Synthetic Procedure for preparation of 1-(Aryl)-3-methyl-3-aza-bicyclo[3.1.0]hexane-2,4-diones

Pursuant to step d of Reaction Scheme 14, trimethylsulphoxonium chloride (1.2 eq.) and sodium hydride (60% dispersion in mineral oil, 1.2 eq.) were suspended in THF (50 vol) and heated at reflux (66° C.) for 2 hours. The reactions were cooled to 50° C. and a solution of 1-methyl-3-(aryl)pyrrole-2,5-dione (1 eq.) in THF (10 mL) was added in one portion. The reactions were heated at 50° C. for between 2 and 4 hours and then at 65° C. for a further 2 hours if required (as judged by disappearance of starting material by TLC), and then cooled to room temperature. Reactions were quenched by the addition of IMS (5 mL) and the solvents removed in vacuo. The residues were taken up in DCM (35 mL) and washed with water (3×35 mL). Combined aqueous washes were back-extracted with DCM (15 mL), organic portions combined and solvent removed in vacuo. The reactions were purified by column chromatography (30 g silica, eluting with increasingly polar fractions of ethyl acetate in hexanes) and solvents removed in vacuo to give the 3-methyl-1-(aryl)-3-aza-bicyclo[3.1.0]hexane-2,4-diones as crude solids. The compounds shown below (NMR data also listed below) were prepared using the foregoing general procedure:

### (1) 3-Methyl-1-(4-trifluoromethylphenyl)-3-aza-bicyclo[3.1.0]hexane-2,4-dione

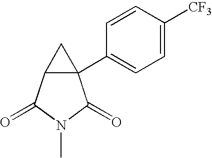

Yield=1.1 g (76%); ¹H NMR (300 MHz, CDCl₃) δ 7.64-7.62 (2H, d, J=8.5 Hz, ArH), 7.55-7.53 (2H, d, J=8.5 Hz, ArH), 2.93 (3H, s, NCH₃), 2.81-2.77 (1H, dd, J=8.7 Hz, 3.7 Hz, CH), 1.92-1.89 (1H, obs t, J=4.3 Hz, CH₂), 1.87-1.83 (1H, dd, J=8.5 Hz, 4.8 Hz, CH₂); MS (m/z) 270 [MH⁺].

### (2) 1-(3-Chlorophenyl)-3-methyl-3-aza-bicyclo[3.1.0]hexane-2,4-dione

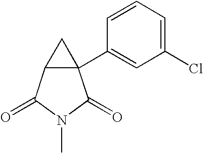

Yield=1.7 g (43%); ¹H NMR (300 MHz, CDCl₃) δ 7.40 (1H, s, ArH), 7.32-7.27 (3H, m, ArH), 2.91 (3H, s, NCH₃), 2.75-2.71 (1H, dd, J=8.1 Hz, 4.0 Hz, CH), 1.89-1.79 (2H, m, CH₂); MS (m/z) 236 [MH⁺].

### (3) 1-(4-Fluorophenyl)-3-methyl-3-aza-bicyclo[3.1.0]hexane-2,4-dione

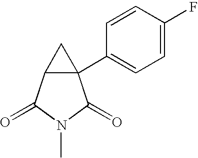

Yield=0.6 g (29%); ¹H NMR (300 MHz, CDCl₃) δ 7.40-7.35 (2H, m, ArH), 7.10-7.03 (2H, m, ArH), 2.92 (3H, s, NCH₃), 2.73-2.69 (1H, dd, J=8.4 Hz, 3.6 Hz, CH), 1.89-1.77 (2H, m, CH₂); MS (m/z) 220 [MH⁺].

### (4) 3-(3-Methyl-2,4-dioxo-3-aza-bicyclo[3.1.0]hex-1-yl)-benzonitrile

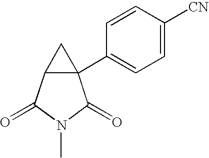

Yield=40 mg (20%); ¹H NMR (300 MHz, CDCl₃) δ 7.69-7.63 (2H, d, J=8.0 Hz, ArH), 7.55-7.52 (2H, d, J=8.4 Hz), 2.91 (3H, s, NCH₃), 2.83-2.79 (1H, dd, J=8.4 Hz, 4.0 Hz, CH), 1.95-1.92 (1H, obs t, J=4.4 Hz, CH₂), 1.86-1.82 (1H, dd, J=8.1 Hz, 4.8 Hz, CH₂); MS (m/z) 227 [MH⁺].

### D. General Synthetic Procedure for Preparation of 1-Aryl-3-methyl-3-aza-bicyclo [3.1.0]hexane hydrochlorides

Pursuant to steps e and f of Reaction Scheme 14, borane (1 M complex in THF, 5 eq.) was cooled to <0° C. and a solution of 3-methyl-1-(aryl)-3-aza-bicyclo[3.1.0]hexane-2,4-dione (1 eq.) in THF (10 vol.) added dropwise, maintaining the temperature <0° C. The reactions were warmed to room temperature for 15 minutes then heated to reflux (67° C.) for 2 hours. The reactions were cooled to <0° C. and quenched with the dropwise addition of 6 M HCl (5 vol., temperature maintained <0° C.). Solvents were removed in vacuo and the resulting white residues basified with the addition of 5 M NaOH (25 mL) and extracted with DCM (2×20 mL). The organics were washed with water (3×30 mL) then concentrated in vacuo to ˜1 mL volume. The resulting oils were purified by column chromatography (15 g silica, eluting with DCM then 5% MeOH in DCM) to give the crude free bases. Samples were dissolved in diethyl ether (1 mL) and 1 M HCl in ether (10 mL) was added. The resulting white precipitates were stored at −20° C. for 16 hours then centrifuged. Ether was decanted and the solids washed with a further three portions of ether (material isolated by centrifugation and ether decanted after each wash). Materials were dried in vacuo at 30° C. to give the required products as white solids. The compounds shown below (NMR data also listed below) were prepared using the general procedures described above:

### (1) 3-Methyl-1-(4-trifluoromethylphenyl)-3-aza-bicyclo[3.1.0]hexane

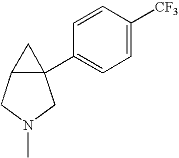

Free base: ¹H NMR (300 MHz, CDCl₃) δ 7.51-7.48 (2H, d, J=8.1 Hz, ArH), 7.20-7.17 (2H, d, J=8.1 Hz, ArH), 3.33-3.30 (1H, d, J=8.4 Hz, CH₂), 3.09-3.06 (1H, d, J=8.4 Hz, CH₂), 2.59-2.56 (1H, d, J=8.4 Hz, CH₂), 2.48-2.44 (1H, dd, J=8.9 Hz, 3.7 Hz, CH₂) 2.36 (3H, s, NCH₃), 1.76-1.71 (1H, m, CH), 1.53-1.50 (1H, obs t, J=4.5 Hz, CH₂) 0.85-0.81 (1H, dd, J=8.1 Hz, 4.4 Hz, CH₂); Hydrochloride salt: Yield=384 mg (34%); ¹H NMR (300 MHz, CDCl₃) δ 12.46 (1H, br-s, N⁺H), 7.57-7.54 (2H, d, J=8.4 Hz, ArH), 7.29-7.26 (2H, d, J=8.4 Hz, ArH), 4.11-4.06 (1H, dd, J=10.8 Hz, 5.0 Hz, CH₂), 3.90-3.85 (1H, dd, J=11.0 Hz, 4.7 Hz, CH₂), 3.44-3.36 (2H, m, CH₂), 2.92-2.91 (3H, d, J=4.4 Hz, NCH₃), 2.27-2.23 (1H, m, CH₂), 2.10-2.05 (1H, m, CH), 1.21-1.16 (1H, obs t, J=7.9 Hz, CH₂); MS (m/z) 242 [MH⁺].

### (2) 1-(3-Chlorophenyl)-3-methyl-3-aza-bicyclo[3.1.0]hexane

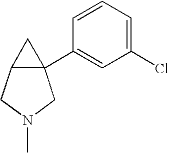

Free base: ¹H NMR (300 MHz, CDCl₃) δ 7.21-7.07 (2H, m, ArH), 6.97-6.94 (1H, dt, J=7.2 Hz, 1.6 Hz, ArH), 3.26-3.23 (1H, d, J=8.4 Hz, CH₂), 3.04-3.01 (1H, d, J=8.8 Hz, CH₂), 2.52-2.49 (1H, d, J=8.8 Hz, CH₂), 2.44-2.40 (1H, dd, J=8.6 Hz, 3.4 Hz, CH₂) 2.32 (3H, s, NCH₃), 1.67-1.60 (1H, m, CH), 1.43-1.38 (1H, m, CH₂) 0.78-0.73 (1H, dd, J=8.1 Hz, 4.4 Hz, CH₂); Hydrochloride salt: Yield=586 mg (33%); ¹H NMR (300 MHz, CDCl₃) δ 12.24 (1H, br-s, N⁺H), 7.30-7.22 (3H, m, ArH), 7.13-7.11 (1H, m, ArH), 4.06-4.01 (1H, dd, J=10.6 Hz, 5.1 Hz, CH₂), 3.88-3.83 (1H, dd, J=10.8 Hz, 5.0 Hz, CH₂), 3.58-3.41 (2H, m, CH₂), 2.97-2.96 (3H, d, J=4.4 Hz, NCH₃), 2.20-2.16 (1H, m, CH₂), 2.07-2.02 (1H, m, CH), 1.21-1.17 (1H, obs t, J=7.5 Hz, CH₂); MS (m/z) 208 [MH⁺] (100), 210 [M(³⁷Cl)H⁺](33).

### (3) 1-(4-Fluorophenyl)-3-methyl-3-aza-bicyclo[3.1.0]hexane

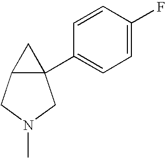

Free base: ¹H NMR (300 MHz, CDCl₃) δ 7.11-7.04 (2H, m, ArH), 7.00-6.88 (2H, m, ArH), 3.26-3.24 (1H, d, J=8.5 Hz, CH₂), 3.06-3.03 (1H, d, J=8.8 Hz, CH₂), 2.51-2.4 (2H, m, CH₂), 2.34 (3H, s, NCH₃), 1.64-1.59 (1H, m, CH), 1.40-1.35 (1H, m, CH₂) 0.75-0.71 (1H, dd, J=7.9 Hz, 4.2 Hz, CH₂); Hydrochloride salt: Yield=166 mg (27%); ¹H NMR (300 MHz, CDCl₃) δ 12.17 (1H, br-s, N⁺H), 7.19-7.14 (2H, m, ArH), 6.94-6.88 (2H, t, J=9.6 Hz, ArH), 3.99-3.93 (1H, dd, J=10.7 Hz, 4.8 Hz, CH₂), 3.82-3.77 (1H, dd, J=10.7 Hz, 4.4 Hz, CH₂), 3.46-3.41 (1H, m, CH₂), 3.32-3.26 (1H, obs t, J=9.4 Hz, CH₂), 2.88-2.87 (3H, d, J=4.0 Hz, NCH₃), 2.09-2.05 (1H, m, CH₂), 1.95-1.91 (1H, m, CH), 1.12-1.06 (1H, obs t, J=7.6 Hz, CH₂); MS (m/z) 192 [MH⁺].

### (4) (4-(3-Methyl-3-aza-bicyclo[3.1.0]hexan-1-yl)phenyl)methanamine

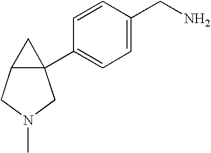

Free base: ¹H NMR (300 MHz, CDCl₃) δ 7.39-7.36 (2H, d, J=8.4 Hz, Ar—H), 7.32-7.29 (2H, d, J=8.4 Hz, Ar—H), 4.02 (2H, s, ArCH₂), 3.95-3.80 (1H, d, J=11.2 Hz, HCH), 3.72-3.65 (1H, d, J=11.2 Hz, HCH), 3.60-3.52 (2H, m, CH₂), 2.90 (3H, s, NCH₃), 2.16-2.06 (1H, q, J=4.2 Hz, CH), 1.39-1.36 (1H, obs-t, J=6.6 Hz, HCH), 1.20-1.14 (1H, obs-t, J=8.4 Hz, HCH); Hydrochloride salt: Yield=15 mg (36%); ¹H NMR (300 MHz, CDCl₃) δ 7.53-7.50 (2H, d, J=8.1 Hz, Ar—H), 7.46-7.43 (2H, d, J=8.4 Hz, Ar—H), 4.16 (2H, s, ArCH₂), 4.12-4.08 (1H, d, J=11.4 Hz, HCH), 3.87-3.84 (1H, d, J=11.1 Hz, HCH), 3.69-3.66 (2H, br-d, J=11.1 Hz, CH₂), 2.28-2.23 (1H, q, J=4.2 Hz, CH), 1.54-1.50 (1H, dd, J=6.9, 4.8 Hz, HCH), 1.31-1.26 (1H, obs-t, J=8.1 Hz, HCH); MS (m/z) 203 [MH⁺].

### EXAMPLE X

### Preparation of 1-Aryl-3-ethyl-3-aza-bicyclo[3.1.0]hexane Hydrochlorides Using Reaction Scheme 15

### A. Synthesis of 3-Bromo-1-ethylmaleimide

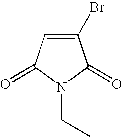

A cooled (5° C.) solution of N-ethylmaleimide (20 g, 0.16 mole) in carbon tetrachloride (20 mL) under nitrogen was treated dropwise over 45 min with bromine (23 g, 0.14 mole) at a rate to keep pot temp <10° C. The mixture was stirred at 5° C. for 2 hours. Dichloromethane (20 mL) was added to the reaction and N₂ was bubbled through the reaction for 15 min to remove excess bromine. The reaction was blown dry with a steady stream of N₂ and then brought up in ethanol. Anhydrous sodium acetate (12.3 g, 0.15 mole) was added and the reaction was refluxed for 4 hours. The mixture was concentrated in vacuo and the residue taken up in methylene chloride (300 mL), filtered and concentrated in vacuo to yield an orange oil. Pure 3-bromo-1-ethylmaleimide was obtained from recrystallization in chloroform to yield a yellowish solid (26 g, 82%). NO MS (M+1) peak observed. ¹H NMR (CDCl₃) δ 1.20 (t, J=7.22 Hz, 3H) 3.62 (q, J=7.22 Hz, 2H) 6.85 (s, 1H).

### B. Synthesis of 1-(4-Trifluoromethylphenyl)-3-ethyl)-3-azabicyclo[3.1.0]hexane, hydrochloride

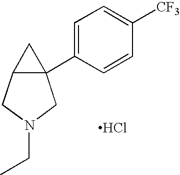

A stirred solution/suspension of 3-bromo-1-ethylmaleimide (1.0 g, 5 mmol) and 4-trifluoromethylphenylboronic acid (1025 mg, 5.4 mmol) in dioxane (15 mL) under nitrogen was degassed with a stream of nitrogen for 10 min, treated with cesium fluoride (1.6 g, 10.8 mmol) and Cl₂Pd(dppf).CH₂Cl₂ (0.25 g, 0.3 mmol), then stirred at room temperature for 1 h and at 40° C. for 45 min. The mixture was then cooled and diluted with methylene chloride (50 mL). The mixture was filtered through Celite® (rinse filter cake with methylene chloride) and the brown filtrate concentrated in vacuo. The residue was dissolved in methylene chloride and filtered through a column of silica gel (eluted with methylene chloride) to afford a pale yellow solid, which was triturated from cold petroleum ethers to afford arylmaleimide intermediate (994 mg, 75%) as a pale yellow solid.

A stirred suspension of sodium hydride oil dispersion (60%, 145 mg, 3.7 mmol) in anhydrous tetrahydrofuran (30 mL) under nitrogen was treated with trimethyl-sulfoxonium chloride (0.52 g, 4.06 mmol), then refluxed for 2.5 h and cooled (50° C.). The above arylmaleimide (994 mg, 3.7 mmol) was added in one portion and the mixture stirred at 50° C. for 3 h, cooled on an ice bath, and quenched with saturated ammonium chloride (10 mL). The product mixture was extracted with ether (2×50 mL), and the combined extracts washed with water (30 mL), dried (MgSO₄), and concentrated in vacuo. The residual solid was dissolved in 1:1 methylene chloride/heptane and loaded onto a silica gel column and eluted with 1:1, 2:1, then 3:1 methylene chloride/heptane to afford bicyclic diimide intermediate (713 mg, 68%) as a very pale yellow oil. ¹H NMR (CDCl₃) δ 1.14 (t, 3H) 1.79-1.88 (m, 2H) 2.78 (dd, J=7.42, 4.49 Hz, 1H) 3.43-3.55 (m, 2H) 7.60 (dd, 4H).

A stirred ice-cooled solution of 1.0 N borane/THF (16 mL, 16 mmol) under nitrogen was treated dropwise with a solution of the above bicyclic diimide intermediate (700 mg, 2.47 mmol) in anhydrous THF (10 mL). The solution was stirred at room temperature for 15 min, refluxed for 4 h, cooled on an ice bath, and carefully treated dropwise with 6 N HCl (10 mL, vigorous evolution of gas). The solution was concentrated to a white solid, which was partitioned between 5 N sodium hydroxide (25 mL) and ether (50 mL). The organic layer was separated and the aqueous extracted with ether (50 mL). The combined organic solution was washed with water (25 mL), dried (Mg₂SO₄), and concentrated in vacuo. The residue was dissolved in methanol (23 mL), treated with 4 N HCl/dioxane (7 mL), then stirred at room temperature for 16 h and at 55° C. for 4 h. The solution was concentrated in vacuo and the residue triturated from ether to afford 1-(4-trifluoromethylphenyl)-3-ethyl)-3-azabicyclo[3.1.0]hexane, hydrochloride (300 mg, 50%) as a white solid. MS (M+1) 256. ¹H NMR (CDCl₃) δ 1.22 (t, J=7.81 Hz, 1H) 1.53 (t, J=7.32 Hz, 3H) 2.04-2.14 (m, 4H) 2.44 (dd, J=6.83, 4.88 Hz, 4H) 3.12-3.31 (m, 4H) 3.95 (dd, J=11.03, 5.37 Hz, 1H) 4.17 (dd, J=10.84, 5.37 Hz, 1H) 7.27 (d, 2H) 7.60 (d, J=8.20 Hz, 2H). ³C NMR (CDCl₃) δ 158.83, 156.34, 135.62, 129.93, 127.57, 121.54, 117.17, 59.78, 57.35, 53.99, 30.68, 23.06, 19.05, 16.29.

### C. Synthesis of 1-(4-Methyoxyphenyl)-3-ethyl-3-azabicyclo[3.1.0]hexane, hydrochloride

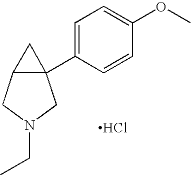

A stirred solution/suspension of 3-bromo-1-ethylmaleimide (1.0 g, 5 mmol) and 4-methoxyphenylboronic acid (820 mg, 5.4 mmol) in dioxane (15 mL) under nitrogen was degassed with a stream of nitrogen for 10 min, treated with cesium fluoride (1.6 g, 10.8 mmol) and Cl₂Pd(dppf).CH₂Cl₂ (0.25 g, 0.3 mmol), then stirred at room temperature for 1 h and at 40° C. for 45 min. The mixture was then cooled and diluted with methylene chloride (50 mL). The mixture was filtered through Celite® (rinse filter cake with methylene chloride) and the brown filtrate concentrated in vacuo. The residue was dissolved in methylene chloride and filtered through a column of silica gel (eluted with methylene chloride) to afford a pale yellow solid, which was triturated from cold petroleum ethers to afford arylmaleimide intermediate (969 mg, 86%) as a pale yellow solid.

A stirred suspension of sodium hydride oil dispersion (60%, 164 mg, 4.19 mmol) in anhydrous tetrahydrofuran (30 mL) under nitrogen was treated with trimethyl-sulfoxonium chloride (0.59 g, 4.61 mmol), then refluxed for 2.5 h and cooled (50° C.). The above arylmaleimide (969 mg, 4.19 mmol) was added in one portion and the mixture stirred at 50° C. for 3 h, cooled on an ice bath, and quenched with saturated ammonium chloride (10 mL). The product mixture was extracted with ether (2×50 mL), and the combined extracts washed with water (30 mL), dried (MgSO₄), and concentrated in vacuo. The residual solid was dissolved in 1:1 methylene chloride/heptane and loaded onto a silica gel column and eluted with 1:1, 2:1, then 3:1 methylene chloride/heptane to afford bicyclic diimide intermediate (334 mg, 33%) as a very pale yellow oil. ¹H NMR (CDCl₃) δ 1.12 (t, J=7.13 Hz, 3H) 1.68-1.84 (m, 2H) 2.65 (dd, J=8.00, 3.71 Hz, 1H) 3.32-3.53 (m, 2H) 3.80 (s, 3H) 6.90 (d, J=8.79 Hz, 2H) 7.31 (d, J=8.79 Hz, 2H).

A stirred ice-cooled solution of 1.0 N borane/THF (16 mL, 16 mmol) under nitrogen was treated dropwise with a solution of the above bicyclic diimide intermediate (330 mg, 1.35 mmol) in anhydrous THF (10 mL). The solution was stirred at room temperature for 15 min, refluxed for 4 h, cooled on an ice bath, and carefully treated dropwise with 6 N HCl (10 mL, vigorous evolution of gas). The solution was concentrated to a white solid, which was partitioned between 5 N sodium hydroxide (25 mL) and ether (50 mL). The organic layer was separated and the aqueous extracted with ether (50 mL). The combined organic solution was washed with water (25 mL), dried (Mg₂SO₄), and concentrated in vacuo. The residue was dissolved in methanol (23 mL), treated with 4 N HCl/dioxane (7 mL), then stirred at room temperature for 16 h and at 55° C. for 4 h. The solution was concentrated in vacuo and the residue triturated from ether to afford 1-(4-methyoxyphenyl)-3-ethyl-3-azabicyclo[3.1.0]hexane, hydrochloride

(210 mg, 40%) as a white solid. MS (M+1) 218. ¹H NMR (CDCl₃) δ 1.11 (t, J=7.52 Hz, 1H) 1.47 (t, J=6.93 Hz, 3H) 1.86-1.94 (m, 1H) 2.17 (dd, J=6.54, 4.59 Hz, 1H) 3.09-3.22 (m, 2H) 3.22-3.33 (m, J=7.22, 7.22 Hz, 2H) 3.73-3.78 (m, 3H) 3.87 (dd, J=10.74, 5.08 Hz, 1H) 4.03 (dd, J=10.84, 5.17 Hz, 1H) 6.83 (d, J=8.59 Hz, 2H) 7.11 (d, J=8.59 Hz, 2H). ¹³CNMR(CDCl₃) δ9.72, 15.84, 22.47, 30.76, 51.48, 55.55, 59.10, 114.51, 128.85, 130.05, 159.19.

### D. Synthesis of 1-(4-Fluorophenyl)-3-ethyl-3-azabicyclo[3.1.0]hexane, hydrochloride

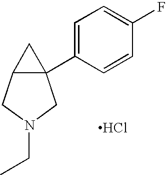

A stirred solution/suspension of 3-bromo-1-ethylmaleimide (1.0 g, 5 mmol) and 4-fluorophenylboronic acid (755 mg, 5.4 mmol) in dioxane (15 mL) under nitrogen was degassed with a stream of nitrogen for 10 min, treated with cesium fluoride (1.6 g, 10.8 mmol) and Cl₂Pd(dppf).CH₂Cl₂ (0.25 g, 0.3 mmol), then stirred at room temperature for 1 h and at 40° C. for 45 min. The mixture was then cooled and diluted with methylene chloride (50 mL). The mixture was filtered through Celite® (rinse filter cake with methylene chloride) and the brown filtrate concentrated in vacuo. The residue was dissolved in methylene chloride and filtered through a column of silica gel (eluted with methylene chloride) to afford a pale yellow solid, which was triturated from cold petroleum ethers to afford arylmaleimide intermediate (808 mg, 75%) as a pale yellow solid.

A stirred suspension of sodium hydride oil dispersion (60%, 147 mg, 3.6 mmol) in anhydrous tetrahydrofuran (30 mL) under nitrogen was treated with trimethyl-sulfoxonium chloride (0.52 g, 4.05 mmol), then refluxed for 2.5 h and cooled (50° C.). The above arylmaleimide (807 mg, 3.68 mmol) was added in one portion and the mixture stirred at 50° C. for 3 h, cooled on an ice bath, and quenched with saturated ammonium chloride (10 mL). The product mixture was extracted with ether (2×50 mL), and the combined extracts washed with water (30 mL), dried (MgSO₄), and concentrated in vacuo. The residual solid was dissolved in 1:1 methylene chloride/heptane and loaded onto a silica gel column and eluted with 1:1, 2:1, then 3:1 methylene chloride/heptane to afford bicyclic diimide intermediate (284 mg, 33%) as a very pale yellow oil. ¹H NMR (CDCl₃) δ 1.13 (t, J=7.22 Hz, 3H) 1.74-1.83 (m, 2H) 2.64-2.73 (m, 1H) 3.42-3.55 (m, 2H) 7.07 (t, J=8.69 Hz, 2H) 7.38 (dd, J=8.79, 5.27 Hz, 2H).

A stirred ice-cooled solution of 1.0 N borane/THF (8.4 mL, 8.4 mmol) under nitrogen was treated dropwise with a solution of the above bicyclic diimide intermediate (283 mg, 1.2 mmol) in anhydrous THF (10 mL). The solution was stirred at room temperature for 15 min, refluxed for 4 h, cooled on an ice bath, and carefully treated dropwise with 6N HCl (10 mL, vigorous evolution of gas). The solution was concentrated to a white solid, which was partitioned between 5 N sodium hydroxide (25 mL) and ether (50 mL). The organic layer was separated and the aqueous extracted with ether (50 mL). The combined organic solution was washed with water (25 mL), dried (Mg₂SO₄), and concentrated in vacuo. The residue was dissolved in methanol (23 mL), treated with 4 N HCl/dioxane (7 mL), then stirred at room temperature for 16 h and at 55° C. for 4 h. The solution was concentrated in vacuo and the residue triturated from ether to afford 1-(4-fluorophenyl)-3-ethyl-3-azabicyclo[3.1.0]hexane, hydrochloride (105 mg, 43%) as a white solid. MS (M+1) 206. ¹H NMR (CDCl₃) δ 1.15 (t, 1H) 1.51 (t, J=7.26 Hz, 3H) 1.93-2.00 (m, 1H) 2.31 (dd, J=6.76, 4.64 Hz, 1H) 3.09-3.29 (m, 4H) 3.92 (dd, J=110.89, 5.34 Hz, 1H) 4.10 (dd, J=10.89, 5.34 Hz, 1H) 6.99-7.06 (m, 2H) 7.12-7.19 (m, 2H). ¹³C NMR (CDCl₃) δ 158.83, 156.34, 135.62, 129.93, 127.57, 121.54, 117.17, 59.78, 57.35, 53.99, 30.68, 23.06, 19.05, 16.29.

### E. Synthesis of 1-(Biphenyl-4-yl)-3-ethyl-3-azabicyclo[3.1.0]hexane, hydrochloride

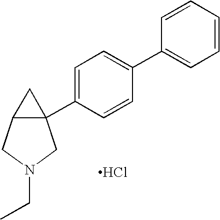

A stirred solution/suspension of 3-bromo-1-ethylmaleimide (0.7 g, 3.43 mmol) and biphenyl-4-ylboronic acid (1.2 g, 5.9 mmol) in dioxane (15 mL) under nitrogen was degassed with a stream of nitrogen for 10 min, treated with cesium fluoride (1.6 g, 10.8 mmol) and Cl₂Pd(dppf).CH₂Cl₂ (0.25 g, 0.3 mmol), then stirred at room temperature for 0.5 h and at 45° C. for 30 min then at 65° C. for 45 min. The mixture was cooled and diluted with methylene chloride (50 mL). The mixture was filtered through Celite® (rinse filter cake with methylene chloride) and the brown filtrate concentrated in vacuo. The residue was dissolved in methylene chloride and filtered through a column of silica gel (eluted with methylene chloride 60% and ethyl acetate 40%) to afford a yellowish solid, which was triturated from cold petroleum ethers to afford arylmaleimide intermediate (1.4 g, 72%) as yellowish solid.

A stirred suspension of sodium hydride oil dispersion (60%, 203 mg, 5.05 mmol) in anhydrous tetrahydrofuran (30 mL) under nitrogen was treated with trimethyl-sulfoxonium chloride (0.715 g, 5.56 mmol), then refluxed for 2.5 h and cooled (50° C.). The above arylmaleimide (1.4 g, 5.05 mmol) was added in one portion and the mixture stirred at 50° C. for 3 h, cooled on an ice bath, and quenched with saturated ammonium chloride (10 mL). The product mixture was extracted with ether (2×50 mL), and the combined extracts washed with water (30 mL), dried (MgSO₄), and concentrated in vacuo. The residual solid was dissolved in 1:1 methylene chloride/heptane and loaded onto a silica gel column and eluted with 1:1, 2:1, then 3:1 methylene chloride/heptane to afford bicyclic diimide intermediate (416 mg, 28%) as a very pale yellow oil. ¹H NMR (CDCl₃) δ 1.15 (t, J=7.22 Hz, 3H) 1.78-1.85 (m, 1H) 1.88 (dd, J=8.20, 4.49 Hz, 1H) 2.74 (dd, J=8.20, 3.71 Hz, 1H) 3.39-3.58 (m, 2H) 7.31-7.39 (m, 1H) 7.39-7.51 (m, 4H) 7.53-7.63 (m, 4H).

A stirred ice-cooled solution of 1.0 N borane/THF (12 mL, 12 mmol) under nitrogen was treated dropwise with a solution of the above bicyclic diimide intermediate (450 mg, 1.5 mmol) in anhydrous THF (10 mL). The solution was stirred at room temperature for 15 min, refluxed for 4 h, cooled on an ice bath, and carefully treated dropwise with 6N HCl (10 mL, vigorous evolution of gas). The solution was concentrated to a white solid, which was partitioned between 5 N sodium hydroxide (25 mL) and ether (50 mL). The organic layer was separated and the aqueous extracted with ether (50 mL). The combined organic solution was washed with water (25 mL), dried (Mg₂SO₄), and concentrated in vacuo. The residue was dissolved in methanol (23 mL), treated with 4 N HCl/dioxane (7 mL), then stirred at room temperature for 16 h and at 55° C. for 4 h. The solution was concentrated in vacuo and the residue triturated from ether to afford 1-(biphenyl-4-yl)-3-ethyl-3-azabicyclo[3.1.0]hexane, hydrochloride (110 mg, 30%) as a white solid. MS (M+1) 264.1. ¹H NMR (CDCl₃) δ 1.21 (t, J=7.61 Hz, 1H) 1.50 (t, J=7.13 Hz, 3H) 1.97-2.08 (m, 1H) 2.29 (dd, J=6.64, 4.69 Hz, 1H) 3.12-3.36 (m, 4H) 3.91 (dd, J=10.84, 5.17 Hz, 1H) 4.12 (dd, J=10.74, 5.27 Hz, 1H) 7.19-7.26 (m, 2H) 7.29-7.38 (m, 1H) 7.37-7.46 (m, 2H) 7.48-7.58 (m, 4H). ¹³C NMR (CDCl₃) 811.16, 16.67, 23.00, 30.94, 51.55, 55.39, 58.56, 127.19, 127.62, 127.67, 129.09, 137.30, 140.45, 140.69.

### EXAMPLE XI

### Preparation of 1-Aryl-3-isopropyl-3-aza-bicyclo[3.1.0]hexane hydrochlorides Using Reaction Scheme 16

### A. Synthesis of 3-Bromo-1-(1-methylethyl)maleimide

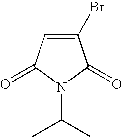

A cooled (5° C.) stirred solution of maleic anhydride (29.4 g, 0.30 mole) in anhydrous ether (150 mL) under nitrogen was treated dropwise over 45 min with a solution of isopropylamine (35.5 g, 0.60 mole) in anhydrous ether (100 mL) at a rate to keep the pot temp <20° C., then the mixture was stirred at 110° C. for 15 min, filtered, and the filter cake rinsed with anhydrous ether and dried in vacuo to afford a white solid. This was taken up in acetic anhydride (250 mL), treated with anhydrous sodium acetate (12.3 g, 0.15 mole), and heated to 75° C. with stirring for 4.5 h, then at 100° C. for 1.5 h. The mixture was concentrated in vacuo and the residue taken up in methylene chloride (300 mL), washed with saturated aqueous sodium bicarbonate (200 mL), water (200 mL), dried (MgSO₄), and concentrated in vacuo. The residue was distilled (approx. 5 mm pressure) to afford two products; one a N-isopropylmaleimide at 82° C. (13.0 g), the other an acetate adduct of N-isopropylmaleimide at 154° C. (12.9 g). The acetate adduct was dissolved in 4:1 acetonitrile/triethylamine (100 mL), heated to 65° C. for 4 h, then concentrated in vacuo. The residue was dissolved in methylene chloride and filtered through a pad of silica gel (eluted with methylene chloride) to afford an additional 3.5 g of N-isopropylmaleimide. Total yield was 16.5 g of N-isopropylmaleimide (40%).

A stirred ice-cold solution of N-isopropylmaleimide (16.4 g, 0.118 mole) in carbon tetrachloride (12 mL) under nitrogen was treated dropwise with bromine (6.41 mL, 0.25 mole) at a rate to keep the pot temp <9° C., then stirred at 3° C. for 2 h, during which time the mixture formed a solid cake. The cake was maintained under a stream of nitrogen to allow excess bromine and CCl₄ to evaporate, then the reaction mixture was placed under vacuum to remove the remaining solvent. Ethanol (100 mL) was added to the flask, followed by sodium acetate (11 g, 0.134 mole), and the mixture was refluxed for 16 h with stirring. The cooled solution was filtered through Celite® (filter cake rinsed with methylene chloride), and the filtrate concentrated in vacuo, dissolved in methylene chloride, filtered through a pad of alumina (eluted with methylene chloride), and re-concentrated in vacuo. The residue was dissolved in 2:1 petroleum ether/methylene chloride, loaded onto a column of silica gel, and eluted successively with 2:1 petroleum ethers/CH₂Cl₂, 1:1 petroleum ethers/CH₂Cl₂, and CH₂Cl₂ alone to afford the subject compound (16.45 g, 64% yield) as a pale yellow, low melting solid.

No MS (M+1) peak observed. ¹H NMR (CDCl₃) δ 6.78 (s, 1H), 4.30-4.40 (m, 1H), 1.37 (d, 6H, J=8 Hz))

### B. Synthesis of 1-(4-(Trifluoromethyl)phenyl)-3-isopropyl-azabicyclo[3.1.0]hexane, hydrochloride

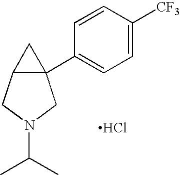

A stirred solution/suspension of 3-bromo-1-(1-methylethyl)maleimide (1.09 g, 5 mmol) and 4-(trifluoromethyl)phenylboronic acid (1.09 g, 6.25 mmol) in dioxane (15 mL) under nitrogen was degassed with a stream of nitrogen for 10 min, treated with cesium fluoride (1.8 g, 11.8 mmol) and Cl₂Pd(dppf).CH₂Cl₂ (0.25 g, 0.3 mmol), then stirred at room temperature for 1 h and at 40° C. for 1 h. The mixture was then cooled and diluted with methylene chloride (50 mL). The mixture was filtered through Celite® (rinse filter cake with methylene chloride) and the brown filtrate concentrated in vacuo. The residue was dissolved in methylene chloride and filtered through a column of silica gel (eluted with methylene chloride) to afford a yellow solid, which was triturated from cold petroleum ethers to afford arylmaleimide intermediate (1.12 g, 79%) as a very pale yellow solid. No MS (M+1) peak. ¹H NMR (CDCl₃) δ 8.01 (d, 2H, J=8 Hz), 7.70 (d, 2H, J=8 Hz), 6.76 (s, 1H), 4.41 (m, 1H), 1.44 (d, 6H, J=7 Hz).

A stirred suspension of sodium hydride oil dispersion (60%, 140 mg, 3.5 mmol) in anhydrous tetrahydrofuran (30 mL) under nitrogen was treated with trimethyl-sulfoxonium chloride (0.55 g, 4.25 mmol), then refluxed for 2.5 h and cooled (50° C.). The above arylmaleimide (990 mg, 3.5 mmol) was added in one portion and the mixture stirred at 50° C. for 3 h, cooled on an ice bath, and quenched with saturated ammonium chloride (10 mL). The product mixture was extracted with ether (2×50 mL), and the combined extracts washed with water (30 mL), dried (MgSO₄), and concentrated in vacuo. The residual solid was dissolved in 1:1 methylene chloride/heptane and loaded onto a silica gel column and eluted with 1:1, then 3:1 methylene chloride/heptane to afford bicyclic diimide intermediate (777 mg, 75%) as a white solid. No MS (M+1) peak. ¹H NMR (CDCl₃ δ 7.64 (d, 2H, J=8 Hz), 7.55 (d, 2H, J=8 Hz), 4.26 (m, 1H), 2.74 (m, 1H), 1.80 (m, 2H), 1.36 (m, 6H).

A stirred ice-cooled solution of 1.0N borane/THF (17.5 mL, 17.5 mmol) under nitrogen was treated dropwise with a solution of the above bicyclic diimide intermediate (743 mg, 2.5 mmol) in anhydrous THF (10 mL). The solution was stirred at room temperature for 15 min, refluxed for 4 h, cooled on an ice bath, and carefully treated dropwise with 6 N HCl (10 mL, vigorous evolution of gas). The solution was concentrated to a white solid, which was partitioned between 5 N sodium hydroxide (25 mL) and ether (50 mL). The organic layer was separated and the aqueous extracted with ether (50 mL). The combined organic solution was washed with water (2×30 mL), dried (Mg₂SO₄), and concentrated in vacuo. The residue was dissolved in methanol (23 mL), treated with 4 N HCl/dioxane (7 mL), then stirred at room temperature for 14 h and at 55° C. for 4 h. The solution was concentrated in vacuo and the residue triturated from ether to afford 1-(4-(trifluoromethyl)phenyl)-3-(2-propyl)-3-azabicyclo[3.1.0]hexane, hydrochloride (657 mg, 86%) as a white solid. MS (M+1) 270.2. ¹H NMR (CDCl₃) δ 7.59 (d, 2H, J=8 Hz), 7.27 (d, 2H, J=8 Hz), 4.12 (m, 1H), 3.90 (m, 1H), 3.30 (m, 3H), 2.52 (m, 1H), 2.08 (m, 1H), 1.54 (m, 6H), 1.17 (m, 1H). ¹³C NMR (CDCl₃) δ 142.64, 127.36, 125.92, 125.02, 59.64, 56.69, 53.80, 30.91, 23.38, 18.92, 17.00.

### C. Synthesis of 1-(4-Methoxyphenyl)-3-isopropyl-3-azabicyclo[3.1.0]hexane, hydrochloride

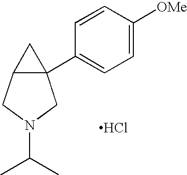

A stirred solution/suspension of 3-bromo-1-(1-methylethyl)maleimide (2.18 g, 10 mmol) and 4-methoxyphenylboronic acid (1.67 g, 11 mmol) in dioxane (30 mL) under nitrogen was degassed with a stream of nitrogen for 10 min, treated with cesium fluoride (3.6 g, 23.7 mmol) and Cl₂Pd(dppf).CH₂Cl₂ (0.50 g, 0.61 mmol), then stirred at room temperature for 1 h and at 40° C. for 5 h, and the mixture was cooled and diluted with methylene chloride (50 mL). The mixture was filtered through Celite® (rinse filter cake with methylene chloride) and the brown filtrate concentrated in vacuo. The residue was dissolved in methylene chloride and filtered through a column of silica gel (eluted with methylene chloride) to afford a solid, which was triturated from cold petroleum ethers to afford arylmaleimide intermediate (1.90 g, 78%) as a bright yellow solid. MS (M+1) 246.2. ¹H NMR (CDCl₃) δ 7.90 (d, 2H, J=9 Hz), 6.95 (d, 2H, J=9 Hz), 6.52 (s, 1H), 4.38 (m, 1H), 3.85 (s, 3H), 1.42 (d, 6H, J=7 Hz).

A stirred suspension of sodium hydride oil dispersion (60%, 180 mg, 4.5 mmol) in anhydrous tetrahydrofuran (30 mL) under nitrogen was treated with trimethyl-sulfoxonium chloride (0.64 g, 5.0 mmol), then refluxed for 2.5 h and cooled (50° C.). The above arylmaleimide (981 mg, 4.0 mmol) was added in one portion and the mixture stirred at 50° C. for 3 h, cooled on an ice bath, and quenched with saturated ammonium chloride (10 mL). The product mixture was extracted with ether (2×50 mL), and the combined extracts washed with water (30 mL), dried (MgSO₄), and concentrated in vacuo. The residual solid was dissolved in petroleum ethers containing a little methylene chloride and loaded onto a silica gel column and eluted with 20% ethyl acetate/petroleum ethers to afford bicyclic diimide intermediate (400 mg, 39%) as a yellow oil. MS (M+1) 260.2. ¹H NMR (CDCl₃) δ 7.31 (d, 2H, J=9 Hz), 6.90 (d, 2H, J=9 Hz), 4.23 (m, 1H), 3.80 (s, 3H), 2.61 (m, 1H), 1.73 (m, 1H), 1.69 (m, 1H), 1.34 (m, 6H).

A stirred ice-cooled solution of 1.0N borane/THF (10 mL, 10 mmol) under nitrogen was treated dropwise with a solution of the above bicyclic diimide intermediate (389 mg, 1.5 mmol) in anhydrous THF (10 mL). The solution was stirred at room temperature for 15 min, refluxed for 3 h, cooled on an ice bath, and carefully treated dropwise with 6 N HCl (4.5 mL, vigorous evolution of gas). The solution was concentrated to a white solid, which was partitioned between 5 N sodium hydroxide (15 mL) and ether (40 mL). The organic layer was separated and the aqueous extracted with ether (40 mL). The combined organic solution was washed with water (20 mL), dried (Mg₂SO₄), and concentrated in vacuo. The residue was dissolved in methanol (15 mL), treated with 4 N HCl/dioxane (4 mL), then stirred at room temperature for 14 h and at 55° C. for 4 h. The solution was concentrated in vacuo and the residue triturated from ether to afford 1-(4-methoxyphenyl)-3-(2-propyl)-3-azabicyclo[3.1.0]hexane, hydrochloride (289 mg, 72%) as a white solid. MS (M+1) 232.2. ¹H NMR (CDCl₃) δ 7.12 (d, 2H, J=9 Hz), 6.86 (d, 2H, J=9 Hz), 4.03 (m, 1H), 3.86 (m, 1H), 3.78 (s, 3H), 3.27 (m, 2H), 3.17 (m, 1H), 2.31 (m, 1H), 1.91 (m, 1H), 1.52 (m, 6H), 1.10 (m, 1H). ¹³C NMR (CDCl₃ δ 159.05, 130.19, 128.78, 114.39, 59.49, 57.82, 55.45, 54.08, 30.82, 22.47, 18.83, 15.71.

### D. Synthesis of 1-(4-Fluorophenyl)-3-isopropyl-3-azabicyclo[3.1.0]hexane, hydrochloride

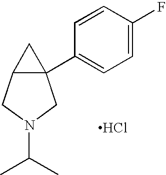

A stirred solution/suspension of 3-bromo-1-(1-methylethyl)maleimide (1.09 g, 5 mmol) and 4-fluorophenylboronic acid (875 mg, 6.25 mmol) in dioxane (15 mL) under nitrogen was degassed with a stream of nitrogen for 10 min, treated with cesium fluoride (1.8 g, 11.8 mmol) and Cl₂Pd(dppf).CH₂Cl₂ (0.25 g, 0.3 mmol), then stirred at room temperature for 1 h and at 40° C. for 3 h, and the mixture was cooled and diluted with methylene chloride (50 mL). The mixture was filtered through Celite® (rinse filter cake with methylene chloride) and the brown filtrate concentrated in vacuo. The residue was dissolved in methylene chloride and filtered through a column of silica gel (eluted with methylene chloride) to afford a pale yellow solid, which was triturated from cold petroleum ethers to afford arylmaleimide intermediate (973 mg, 83%) as a white solid. No MS (M+1) peak. ¹H NMR (CDCl₃) δ 7.92 (m, 2H), 7.13 (m, 2H), 6.61 (s, 1H), 4.39 (m, 1H), 1.43 (d, 6H, J=7 Hz).

A stirred suspension of sodium hydride oil dispersion (60%, 140 mg, 3.5 mmol) in anhydrous tetrahydrofuran (30 mL) under nitrogen was treated with trimethyl-sulfoxonium chloride (0.55 g, 4.25 mmol), then refluxed for 2.5 h and cooled (50° C.). The above arylmaleimide (816 mg, 3.5 mmol) was added in one portion and the mixture stirred at 50° C. for 3 h, cooled on an ice bath, and quenched with saturated ammonium chloride (10 mL). The product mixture was extracted with ether (2×50 mL), and the combined extracts washed with water (30 mL), dried (MgSO₄), and concentrated in vacuo. The residual oil was dissolved in 1:1 methylene chloride/heptane and loaded onto a silica gel column and eluted with 20% ethyl acetate/heptane to afford a white solid, which was triturated from petroleum ethers to afford bicyclic diimide intermediate (482 mg, 56%) as a white solid. No MS (M+1) peak. ¹H NMR (CDCl₃) δ 7.37 (m, 2H), 7.05 (m, 2H), 4.24 (m, 1H), 2.66 (m, 1H), 1.73 (m, 2H), 1.34 (m, 6H).

A stirred ice-cooled solution of 1.0N borane/THF (14 mL, 14 mmol) under nitrogen was treated dropwise with a solution of the above bicyclic diimide intermediate (476 mg, 1.925 mmol) in anhydrous THF (10 mL). The solution was stirred at room temperature for 15 min, refluxed for 4 h, cooled on an ice bath, and carefully treated dropwise with 6 N HCl (7 m]L, vigorous evolution of gas). The solution was concentrated to a white solid, which was partitioned between 5 N sodium hydroxide (25 mL) and ether (50 mL). The organic layer was separated and the aqueous extracted with ether (50 mL). The combined organic solution was washed with water (30 mL), dried (Mg₂SO₄), and concentrated in vacuo. The residue was dissolved in methanol (16 mL), treated with 4N HCl/dioxane (4 mL), then stirred at room temperature for 14 h and at 55° C. for 2.5 h. The solution was concentrated in vacuo and the residue triturated from ether to afford 1-(4-fluorophenyl)-3-(2-propyl)-3-azabicyclo[3.1.0]hexane, hydrochloride (394 mg, 80%) as a white solid. MS (M+1) 220.2. ¹H NMR (CDCl₃) δ 7.17 (m, 2H), 7.02 (m, 2H), 4.05 (m, 1H), 3.87 (m, 1H), 3.30 (m, 2H), 3.19 (m, 1H), 2.38 (m, 1H), 1.95 (m, 1H), 1.53 (d, 6H, J=6 Hz), 1.11 (m, 1H). ¹³C NMR (CDCl₃) δ 162.93, 160.97, 133.98, 129.29, 115.86, 59.47, 57.54, 54.02, 30.65, 22.65, 18.87, 15.74.

### E. Synthesis of 1-(4-Biphenyl)-3-isopropyl-3-azabicyclo[3.1.0]hexane, hydrochloride

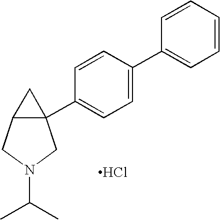

A stirred solution/suspension of 3-bromo-1-(1-methylethyl)maleimide (1.09 g, mmol) and 4-biphenylboronic acid (1.24 g, 6.25 mmol) in dioxane (15 mL) under nitrogen was degassed with a stream of nitrogen for 10 min, treated with cesium fluoride (1.8 g, 11.8 mmol) and Cl₂Pd(dppf).CH₂Cl₂ (0.25 g, 0.3 mmol), then stirred at room temperature for 1 h and at 60° C. for 1 h, and the mixture was cooled and diluted with methylene chloride (50 mL). The mixture was filtered through Celite® (rinse filter cake with methylene chloride) and the brown filtrate concentrated in vacuo. The residue was dissolved in methylene chloride and filtered through a column of silica gel (eluted with methylene chloride) to afford a yellow solid, which was triturated from cold petroleum ethers to afford arylmaleimide intermediate (1.245 g, 86%) as a pale yellow solid. NO MS (M+1) peak. ¹H NMR (CDCl₃) δ 8.00 (m, 2H), 7.68 (m, 2H), 7.63 (m, 2H), 7.47 (m, 2H), 7.39 (m, 1H), 6.69 (s, 1H), 4.42 (m, 1H), 1.45 (d, 6H, J=7 Hz).

A stirred suspension of sodium hydride oil dispersion (60%, 140 mg, 3.5 mmol) in anhydrous tetrahydrofuran (30 mL) under nitrogen was treated with trimethyl-sulfoxonium chloride (0.55 g, 4.25 mmol), then refluxed for 2.5 h and cooled (50° C.). The above arylmaleimide (1.02 g, 3.5 mmol) was added in one portion and the mixture stirred at 50° C. for 3 h, cooled on an ice bath, and quenched with saturated ammonium chloride (10 mL). The product mixture was extracted with ether (2×50 mL), and the combined extracts washed with water (30 mL), dried (MgSO₄), and concentrated in vacuo. The residual solid was dissolved in 1:1 methylene chloride/heptane and loaded onto a silica gel column and eluted with 10% ethyl acetate/heptane, then methylene chloride to afford bicyclic diimide intermediate (933 mg, 87%) as a pale yellow solid. MS (M+1) 306.2 ¹H NMR (CDCl₃) δ 7.59 (m, 4H), 7.40-7.50 (m, 4H), 7.35 (m, 1H), 4.27 (m, 1H), 2.71 (m, 1H), 1.83 (m, 1H), 1.77 (m, 1H), 1.37 (m, 6H).

A stirred ice-cooled solution of 1.0 N borane/THF (17.5 mL, 17.5 mmol) under nitrogen was treated dropwise with a solution of the above bicyclic diimide intermediate (763 mg, 2.5 mmol) in anhydrous THF (10 mL). The solution was stirred at room temperature for 15 min, refluxed for 4 h, cooled on an ice bath, and carefully treated dropwise with 6 N HCl (10 mL, vigorous evolution of gas). The solution was concentrated to a white solid, which was partitioned between 5 N sodium hydroxide (25 mL) and ether (50 mL). The organic layer was separated and the aqueous extracted with ether (50 mL). The combined organic solution was washed with water (2×30 mL), dried (Mg₂SO₄), and concentrated in vacuo. The residue was dissolved in methanol (23 mL), treated with 4 N HCl/dioxane (7 mL), then stirred at room temperature for 14 h and at 55° C. for 4 h. The solution was concentrated in vacuo and the residue triturated from ether to afford 1-(4-biphenyl)-3-(2-propyl)-3-azabicyclo[3.1.0]hexane, hydrochloride (607 mg, 77%) as a white solid. MS (M+1) 278.2. ¹H NMR (CDCl₃) δ 7.55 (m, 4H), 7.43 (m, 2H), 7.34 (m, 1H), 7.24 (m, 2H), 4.11 (m, 1H), 3.89 (m, 1H), 3.25-3.38 (m, 3H), 2.42 (m, 1H), 2.03 (m, 1H), 1.54 (d, 6H, J=7 Hz), 1.19 (m, 1H). ¹³C NMR (CDCl₃) δ 140.23, 140.10, 137.20, 128.72, 127.40, 127.36, 126.81, 59.32, 57.03, 53.77, 30.72, 22.77, 18.72, 18.64, 16.22.

### EXAMPLE XII

### Preparation of 1-(4-Trifluoromethoxyphenyl)-3-azabicyclo[3.0.1]hexane, hydrochloride Using Reaction Scheme 17

### A. Synthesis of 3-Bromo-1-(3,4-dimethoxybenzyl)maleimide

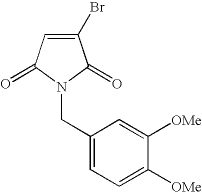

A solution of bromomaleic anhydride (Aldrich, 20.0 g, 0.113 mole) in anhydrous tetrahydrofuran (100 mL) under nitrogen was treated dropwise with a solution of 3,4-dimethoxybenzylamine (20.0 g, 0.1196 mole) in anhydrous THF (40 mL) over 30 min, then the stirred mixture was refluxed for 3 h and maintained at room temperature for 20 h. The mixture was concentrated in vacuo, suspended in acetic anhydride (135 mL), treated with anhydrous sodium acetate (6.15 g, 75 mmol), and heated to 50° C. with stirring under nitrogen for 4 h (solids dissolved after a few minutes). The mixture was concentrated in vacuo and dissolved in methylene chloride (300 mL). The solution was washed with saturated aqueous sodium bicarbonate (150 mL), then with water (150 mL), dried (Na₂SO₄), and concentrated in vacuo to a brown residue. This was dissolved in methylene chloride and passed through a column of silica gel (˜400 mL volume) and eluted with methylene chloride to afford a tan solid, which was recrystallized from ethyl acetate/heptane (2 crops) to afford 3-bromo-1-(3,4-dimethoxybenzyl)maleimide (24.75 g, 67%) as a pale tan solid. NO MS (M+1) peak. ¹H NMR (CDCl₃) δ 6.89-6.94 (m, 2H), 6.84 (s, 1H), 6.78 (d, 1H, J=8 Hz), 4.63 (s, 2H), 3.86 (s, 3H), 3.84 (s, 3H).

### B. Synthesis of 1-(4-Trifluoromethoxyphenyl)-3-azabicyclo[3.1.0]hexane, hydrochloride

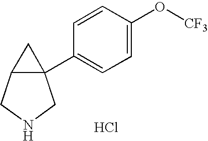

A stirred solution of 3-bromo-1-(3,4-dimethoxybenzyl)maleimide (1.14 g, 3.5 mmol) and 4-(trifluoromethoxy)phenylboronic acid (0.93 g, 4.5 mmol) in anhydrous dioxane (10 mL) under nitrogen was degassed over 10 min with a stream of nitrogen, then treated with cesium fluoride (1.3 g, 8.5 mmol) and Cl₂Pd(dppf).CH₂Cl₂ (Aldrich, 0.17 g, 0.21 mmol), stirred 1 h at room temperature, then 2 h at 40° C. The mixture was cooled, diluted with methylene chloride (50 mL), stirred a few minutes, filtered through Celite® (rinse with methylene chloride), and the filtrate concentrated in vacuo. The residue was dissolved in methylene chloride and loaded onto a silica gel column and the product eluted with 3% ethyl acetate/methylene chloride to afford a yellow solid, which was triturated from petroleum ethers to afford the intermediate arylmaleimide (1.25 g, 88%) as a pale yellow solid. NO MS (M+1) peak. ¹H NMR (CDCl₃) δ 7.96 (d, 2H, J=8.5 Hz), 7.28 (d, 2H, J=8.5 Hz), 6.94-6.99 (m, 2H), 6.80 (m, 1H), 6.73 (s, 1H), 4.67 (s, 2H), 3.87 (s, 3H), 3.85 (s, 3H).

A cooled (−20° C.) stirred solution of trimethylsulfoxonium chloride (515 mg, 4.0 mmol) in anhydrous tetrahydrofuran (15 mL) under nitrogen was treated dropwise with n-butyllithium/hexane (2.4 N, 1.42 mL, 3.4 mmol) and gradually warmed to 50° C. over 30 minutes. Meanwhile, a solution of the intermediate arylmaleimide (1.22 g, 3.0 mmol) in anhydrous THF (10 mL) was heated to 50° C., then added quickly in one portion to the above heated suspension, and the mixture was stirred at 50° C. for 2 h, then cooled on an ice bath. Saturated aqueous ammonium chloride (1 mL) was added to quench, and the mixture was diluted with methylene chloride (75 mL), dried (MgSO₄), filtered through Celite® (rinse with methylene chloride), and concentrated in vacuo. The residue was dissolved in methylene chloride, loaded onto a silica gel column, and the product eluted with 3% ethyl acetate/methylene chloride to afford the intermediate bicyclic diimide (633 mg, 50%) as a very pale yellow viscous oil. MS (M+1) 422.2. ¹H NMR (CDCl₃) δ 7.42 (m, 2H), 7.21 (m, 2H), 6.87-6.93 (m, 2H), 6.79 (m, 1H), 4.51 (m, 2H), 3.86 (s, 3H), 3.84 (s, 3H), 2.74 (m, 1H), 1.77 (m, 1H), 1.72 (m, 1H).

A cooled (5° C.) stirred solution of 1 N lithium aluminum hydride/THF (10 mL, 10 mmol) under nitrogen was treated slowly with a solution of the above intermediate bicyclic diimide (632 mg, 1.5 mmol) in anhydrous THF (7 mL), stirred 1 h at room temperature, refluxed for 6 h, and cooled (5° C.). Water (0.4 mL), 15% sodium hydroxide (0.4 mL), and water (1.2 mL) were carefully added dropwise, followed by additional THF to facilitate stirring. The suspension was stirred 15 min, filtered through Celite® (filter cake rinsed with THF), and the filtrate concentrated in vacuo. The residue was dissolved in methylene chloride, loaded onto a silica gel column, and eluted with 3:1 methylene chloride/ethyl acetate to afford the intermediate dimethoxybenzyl bicyclic amine (302 mg, 51%) as a colorless viscous oil. MS (M+1) 394.3. ¹H NMR (CDCl₃) δ 7.11(m, 4H), 6.88 (m, 1H), 6.81 (m, 2H), 3.88 (s, 3H), 3.86 (s, 3H), 3.60 (m, 2H), 3.24 (m, 1H), 3.05 (m, 1H), 2.55 (m, 2H), 1.69 (m, 1H), 1.53 (m, 1H), 0.78 (m, 1H).

A mixture of the intermediate dimethoxybenzyl bicyclic amine (299 mg, 0.76 mmol) and anhydrous potassium carbonate (225 mg, 1.63 mmol) in anhydrous methylene chloride (5 mL) in a pressure tube equipped with a stir bar was treated with 1-chloroethyl chloroformate (0.225 mL, 1.7 mmol), closed, and stirred at 45° C. for 4 h. The tube was cooled, opened, and the contents filtered (rinse with methylene chloride), and the filtrate concentrated in vacuo. The residue was dissolved in methanol (7 mL), refluxed for 1 h, cooled, treated with DOWEX® 550A-OH resin (2.0 g, prerinsed with methanol), stirred a few minutes, filtered, and the filtrate concentrated in vacuo. The residue was taken up in ether, filtered through Celite®, and the filtrate treated with 2 N HCl/ether (0.6 mL, 1.2 mmol). The suspension was stirred a few minutes, the solid salt collected by filtration, rinsed with ether, and dried in vacuo to afford 1-(4-trifluoromethoxyphenyl)-3-azabicyclo[3.1.0]hexane, hydrochloride (151 mg, 71%) as a light beige solid. MS (M+1) 244.1. ¹H NMR (CDCl₃) δ 10.31 (br s, 1H), 9.83 (br s, 1H), 7.22 (m, 2H), 7.17 (m, 2H), 3.77 (m, 1H), 3.50-3.70 (m, 3H), 1.96 (m, 1H), 1.60 (m, 1H), 1.22 (m, 1H). ³C NMR (CDCl₃) δ 148.61, 136.94, 128.97, 121.62, 50.95, 47.78, 31.10, 23.52, 15.72.

### EXAMPLE XIII

### Preparation of (1S,5R)-5-p-Tolyl-3-aza-bicyclo[3.0.1]hexan-2-one Using Reaction Scheme 18

### A. Preparation of (1R,2S)-2-(hydroxymethyl)-1-p-tolylcyclopropanecarbonitrile

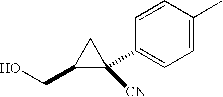

A solution of p-tolylacetonitrile (35.74 g, 0.27 mole) in anhydrous THF (370 mL) was cooled to approximately −20° C. and a 1M solution of sodium hexamethyldisilizide (NaHMDS) (190 mL) was added slowly via addition funnel under nitrogen while keeping the temperature below −10° C. It was stirred at a temperature of −10 to −20° C. for approximately one hour. A solution of (S)-epichlorohydrin (25 g, 0.27 mole) in THF (30 mL) was added slowly via addition funnel and the mixture continued stirring at −10 to −20° C. for 40 min. A second batch of NaHMDS (190 mL) was added in a similar manner and continued with stirring at approximately −20° C. for one hour. The reaction was quenched by addition of water (300 mL) and after stirring the contents for 5 min at ambient temperature, the organic layer was separated, and the aqueous layer was extracted with ethyl acetate (1×350 mL). The combined organic layers were washed with 2M HCl (1×175 mL), brine (1×175 mL), dried over Na₂SO₄, filtered, and concentrated under reduced pressure to give a brown oil. The oil was purified via column chromatography (300 g flash silica) eluting with 5-25% EtOAc in hexanes. The desired fractions were collected, concentrated under reduced pressure, and dried to afford the product as mixture of diastereomers (red oil, 18 g, 35%): ¹H NMR (300 MHz, CDCl₃, peaks corresponding to syn isomer listed) δ: 7.17 (m, 4H, ArH), 4.04 (dd, 1H, CHOH, J=12 Hz and 5.1 Hz), 3.80 (dd, 1H, CHOH, J=12 Hz and 8.4 Hz), 2.33 (s, 3H, CH3), 2.10 (m, 1H, ArCCH₂CH), 1.56 (m, 2H, ArCCH₂CH)

### B. Preparation of ((1S,2R)-2-(Aminomethyl)-2-p-tolylcyclopropyl)methanol

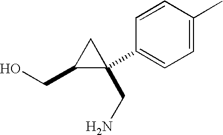

An oven-dried 500 mL round-bottomed flask was charged with LAH (5.68 g, 149.5 mmole), diethyl ether (50 mL), and the resulting mixture was cooled to 5° C. in an ice bath. A solution of carbonitrile from A (14 g, 74.77 mmole) in diethyl ether (100 mL) was added via addition funnel over 1.5 h period, then allowed to warm to ambient temperature overnight. The reaction slurry was cooled to 5° C. and quenched carefully by slow addition of water (6 mL) so that the temp never rose beyond 20° C. To the mixture was added 15% aq NaOH solution (6 mL) followed by water (18 mL). The resulting slurry was stirred at ambient temperature for a couple of hours, filtered, and the filtercake washed with diethyl ether (4×100 mL). The combined filtrates were concentrated under reduced pressure to give the crude amino alcohol as amber oil. The oil was purified via column chromatography using 335 g flash silica and eluting with DCM:MeOH: NH₄OH (20:1:0.1 to 10:1:0.1, v/v/v). The desired fractions were combined, concentrated under reduced pressure, and dried to afford the title compound (5.38 g) as a golden oil (5.38 g, 38%): ¹H NMR (300 MHz, CDCl₃) δ: 7.30 (m, 2H, ArH), 7.14 (m, 2H, ArH), 4.12 (dd, 1H, CHOH, J=12.3 Hz, 5.4 Hz), 3.43 (d, 1H, CHN, 12.3 Hz), 3.34 (dd, 1H, CHOH, J=12.3 Hz, 11.1 Hz), 2.90 (bs, 3H, NH₂, OH), 2.57 (d, 1H, CHN, J=12.3 Hz), 2.33 (s, 3H, ArCH₃), 1.73 (m, 1H, ArCCH₂CH), 0.94 (dd, 1H, ArCCH₂CH, J=8.7 Hz, 4.8 Hz), 0.72 (m, 1H, ARCCH₂CH); [α]_{D}²⁵+49.5 (c=1, MeOH).

### C. Preparation of ((1R,2S)-tert-Butyl-2-(hydroxymethyl)-1-p-tolylcyclopropyl) methylcarbamate

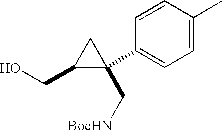

Boc anhydride (6.41 g, 0.029 mole) was added in one portion to a stirred solution of amino alcohol (5.11 g, 0.027 mole) in anhydrous DCM (170 mL). Initially, gas evolution was observed via a bubbler and subsided after a few minutes. Reaction mixture was stirred at ambient temperature for 3 h. The reaction mixture was washed with water (2×100 mL), dried (Na₂SO₄), filtered, and concentrated to give the crude N-boc amino alcohol as yellow syrup. It was purified via column chromatography using approximately 200 g flash silica and eluted with 10-25% EtOAc/hexanes. The desired fractions were combined, concentrated under reduced pressure, and dried to afford the title compound (6.96 g) as colorless glass (6.96 g, 90%): ¹H NMR (300 MHz, CDCl₃) δ: 7.21 (m, 2H, ArH), 7.11 (m, 2H, ArH), 4.81 (bs, 1H, NHBoc), 4.07 (m, 1H), 3.79 (bs, 1H), 3.51 (m, 2H), 3.32 (m, 1H), 2.33 (s, 3H), 1.59 (m, 1H), 1.39 (s, 9H), 0.95(dd, 1H, J=9 Hz, 4.88 Hz), 0.54 (m, 1H); [α]_{D}²⁵+38.9 (c=1, MeOH).

### D. Preparation of (1R,5S)-tert-Butyl 4-oxo-1-p-tolyl-3-aza-bicyclo[3.1.0]hexane-3-carboxylate

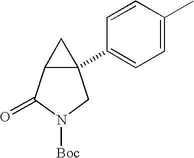

PDC (30.28 g, 0.080 mole) was added in one portion to a stirred solution of N-boc amino alcohol (6.7 g, 0.022 mole) in anhydrous DMF (200 mL). The resulted dark brown reaction mixture was stirred at ambient temperature overnight. The reaction mixture was diluted with water (400 mL) and 2N aq. HCl solution (100 mL) was added. The solution became slightly exothermic. After the solution cooled to ambient temperature, it was extracted with diethyl ether (4×100 mL). The combined organic layer washed with water (2×100 mL), dried (Na₂SO₄), filtered, and concentrated to give the N-boc lactam as a bright white solid (5.59 g, 85%): ¹H NMR (300 MHz, CDCl₃) δ: 7.16 (s, 4H, ArH), 4.01 (dd, 1H, J=11.1 Hz and 1.2 Hz), 3.93 (d, 1H, J=11.4 Hz), 2.35 (s, 3H), 2.24 (m, 1H), 1.58 (m, 1H), 1.52(s, 9H), 1.27 (m, 1H); [α]_{D}²⁵+82.7 (c=1, MeOH).

### E. Preparation of (1S,5R)-5-p-Tolyl-3-aza-bicyclo[3.1.0]hexan-2-one

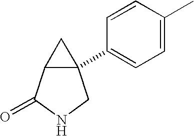

TFA (2.68 mL, 34.8 mmole) was added in one portion with stirring to a colorless solution of N-boc lactam (1.0 g, 3.4 mmole) in anhydrous DCM (25 mL). The resulted light brown solution was stirred at ambient temperature for 1 h. The reaction mixture was concentrated under reduced pressure to give crude product as a light brown syrup. This was purified via column chromatography using approximately 150 g flash silica and eluted with 40-60% EtOAc-hexanes. The desired fractions were combined, concentrated under reduced pressure, and dried to afford the title compound as a bright white solid (1.05 g, 85%): ¹H NMR (300 MHz, CDCl₃) δ: 7.15 (s, 4H, ArH), 6.16 (bs, 1H, NH), 3.67 (s, 2H, —CH₂NH), 2.34 (s, 3H), 2.07 (m, 1H), 1.52 (dd, 1H, J=9 Hz and 4.8 Hz), 1.19 (m, 1H); ¹³C NMR (75 MHz, CDCl₃) δ: 178.74, 137.16, 136.80, 129.53, 127.66, 49.83, 30.65, 27.20, 21.20, 20.03; LC-MS: (+) ESI: m/z=188 [M+1]⁺ (100); UV (λₘₐₓ=254)=97.34%; [α]_{D}²⁵+32.9 (c=1, MeOH); Anal. Calcd for C₁₂H₁₃NO: C, 76.98; H, 7.00; N, 7.48. Found: C, 76.52; H, 16.90; N, 7.47.

### EXAMPLE XIV

### Preparation of (1R,5S)-5-p-Tolyl-3-aza-bicyclo[3.1.0]hexan-2-one Using Reaction Scheme 18

### A. Preparation of (1S,2R)-2-(Hydroxymethyl)-1-p-tolylcyclopropanecarbonitrile

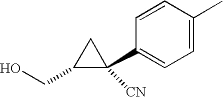

A solution of p-tolyl acetonitrile (25 g, 0.19 mole) in anhydrous THF (180 mL) was cooled to −18° C. and a solution of sodium hexamethyldisilizide (1M THF, 190 mL) was added slowly via addition funnel under nitrogen while keeping the temperature below −10° C. It was stirred at −10 to −20° C. temperature for one additional hour. A solution of (R)-epichlorohydrin (17.6 g, 0.19 mole) in THF (30 mL) was added slowly via addition funnel and continued stirring at −10 to −20° C. for 1.5 h. A second batch of NaHMDS (190 mL) was added slowly and stirring continued while the temperature was maintained at −20° C. for 80 min. The reaction mixture was quenched by addition of water (300 mL). The contents were stirred for 5 min at ambient temperature and the organic layer was separated. The aqueous layer was extracted with ethyl acetate (2×250 mL) and the combined organic layers washed with 2M HCl (1×150 mL), brine (1×150 mL), dried over Na₂SO₄, filtered, and concentrated under reduced pressure to give a reddish-brown oil. The oil was purified via column chromatography (300 g flash silica) with 5-25% EtOAc/hexanes. The desired fractions were collected, concentrated under reduced pressure, and dried under high vacuum to afford the product as mixture of diastereomers (14.9 g, 42%): ¹H NMR (300 MHz, CDCl₃, peaks corresponding to syn isomer listed here) δ: 7.19 (m, 4H, ArH), 4.07 (dd, 1H, CHOH, J=12 Hz and 5.1 Hz), 3.81 (dd, 1H, CHOH, J=12 Hz and 8.4 Hz), 2.34 (s, 3H, CH₃), 2.12 (m, 1H, ArCCH₂CH), 1.57 (m, 2H, ArCCH₂CH)

### B. Preparation of ((1R,2S)-2-(Aminomethyl)-2-p-tolylcyclopropyl)methanol

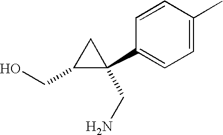

An oven dried and 500 mL round-bottomed flask was charged with LAH (5.68 g, 149.5 mmole) and diethyl ether (50 mL). The reaction mixture was cooled to 5° C. in an ice bath and to this was added a solution of carbonitrile from A (14 g, 74.77 mmole) in diethyl ether (100 mL) via addition funnel over 1.5 h, then warmed to ambient temperature overnight. The reaction slurry was cooled to 5° C. in an ice bath and quenched carefully by slow addition of water (6 mL) so that the temperature never rose beyond 20° C. An aqueous solution of 15% NaOH (6 mL) was added followed by additional water (18 mL). The resulting slurry was stirred at ambient temperature for 2 hours, filtered, and the filtercake washed with diethyl ether (4×100 mL). The combined filtrates were concentrated to give the crude amino alcohol (14.64 g) as a red orange oil. The oil was purified via column chromatography using 385 g flash silica and eluting with DCM:MeOH: NH₄OH (20:1:0.1 to 10:1:0.1; v/v/v). The desired fractions were combined, concentrated under reduced pressure, and dried to afford the title compound as a golden oil. (4.3 g, 30%): ¹H NMR (300 MHz, CDCl₃) δ: 7.30 (m, 2H, ArH), 7.13 (m, 2H, ArH), 4.12 (dd, 1H, CHOH, J=12.3 Hz, 5.4 Hz), 3.43 (dd, 1H, CHN, 12.3 Hz and 0.6 Hz), 3.34 (dd, 1H, CHOH, J=12.3 Hz, 10.8 Hz), 2.97 (bs, 3H, NH₂, OH), 2.57 (d, 1H, CHN, J=12.3 Hz), 2.33 (s, 3H, ArCH₃), 1.72 (m, 1H, ArCCH₂CH), 0.93 (dd, 1H, ArCCH₂CH, J=8.7 Hz, 4.8 Hz), 0.72 (m, 1H, ArCCH₂CH); [α]_{D}²⁵−45.2, (c=1, MeOH).

### C. Preparation of ((1S,2R)-tert-Butyl-2-(hydroxymethyl)-1-p-tolylcyclopropyl) methylcarbamate

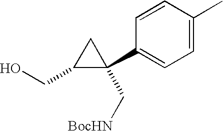

Boc anhydride (65.1 g, 0.023 mole) was added in one portion to a stirred solution of amino alcohol (4.06 g, 0.021 mole) in anhydrous DCM (140 mL). Initially, a gas evolution was observed via an oil-bubbler and subsided after a few minutes. Reaction mixture was stirred at ambient temperature for 3 h. The reaction mixture was washed with water (2×100 mL), dried (Na₂SO₄), filtered, and concentrated to give the crude N-boc amino alcohol as a light yellow syrup. The syrup was purified via column chromatography using approximately 200 g flash silica and eluted with 10-25% EtOAc/hexanes. The desired fractions were combined, concentrated under reduced pressure, and dried under high vacuum to afford the title compound as a light brown glass (4.81 g, 78%): ¹H NMR (300 MHz, CDCl₃) δ: 7.21 (m, 2H, ArH), 7.11 (m, 2H, ArH), 4.77 (bs, 1H), 4.09 (m, 1H), 3.72 (bs, 1H), 3.52 (m, 2H), 3.32 (m, 1H), 2.33 (s, 3H), 1.59 (m, 1H), 1.39 (s, 9H), 0.95 (dd, 1H, J=9 Hz, 4.8 Hz), 0.54 (m, 1H); [α]_{D}²⁵−41.0, (c=1, MeOH).

### D. Preparation of (1S,5R)-tert-Butyl 4-oxo-1-p-tolyl-3-aza-bicyclo[3.1.0]hexane-3-carboxylate

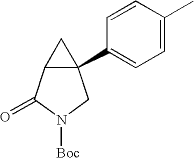

PDC (20.65 g, 0.055 mole) was added in one portion to a stirred solution of N-Boc amino alcohol (4.57 g, 0.016 mole) in anhydrous DMF (135 mL). The resulting dark brown reaction mixture was stirred at ambient temperature overnight. The reaction mixture was diluted with water (300 mL) and 2N aq. HCl (75 mL). The solution became slightly exothermic. Upon cooling, the mixture was extracted with diethyl ether (3×100 mL). The combined organic layers were washed with water (2×100 mL), dried over Na₂SO₄, filtered, and concentrated under reduced pressure to give the N-boc lactam as a bright white solid. The solid was taken up in chloroform (50 mL), dried over Na₂SO₄, filtered, and concentrated under reduced pressure to give a white solid upon drying on high vac (4.3 g, 95%): ¹H NMR (300 MHz, CDCl₃) δ: 7.16 (s, 4H, ArH), 4.01 (dd, 1H, J=11.1 Hz and 1.2 Hz), 3.93 (dd, 1H, J=10.8 Hz and 0.6 Hz), 2.35 (s, 3H), 2.24 (ddd, 1H, J=9 Hz, 3.3 Hz and 1.2 Hz), 1.58 (dd, 1H, J=9 Hz and 4.8 Hz), 1.52(s, 9H), 1.27 (dd, 1H, J=4.8 Hz and 3.3 Hz); [α]_{D}²⁵−79.5, (c=1, MeOH).

### E. Preparation of (1R,5S)-5-p-Tolyl-3-aza-bicyclo[3.1.0]hexan-2-one

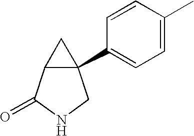

TFA (4.0 mL, 52.2 mmole) was added in one portion to a stirred and colorless solution of N-boc lactam 6N (1.5 g, 5.2 mmole) in anhydrous DCM (30 mL). The resulting light brown solution was stirred at ambient temperature for 1 h. The reaction mixture was concentrated and dried under high vacuum overnight to give the crude product as light yellowish-brown solid. Precipitation from EtOAc gave a white solid that was washed with cold EtOAc and dried under high vacuum to a constant mass to afford the title compound as white solid (0.607 g, 63%): ¹H NMR (300 MHz, CDCl₃) δ: 7.15 (s, 4H, ArH), 6.34 (bs, 1H, NH), 3.68 (s, 2H, —CH NH), 2.34 (s, 3H), 2.07 (ddd, 1H, J=8.7 Hz, 3.3 Hz and 1.5 Hz), 1.52 (dd, 1H, J=8.7 Hz and 4.2 Hz), 1.19 (m, 1H); ¹³C NMR (75 MHz, CDCl₃) δ: 178.74, 137.17, 136.80, 129.54, 127.66, 49.84, 30.67, 27.20, 21.21, 20.03; LC-MS: (+) ESI: m/z=188 [M+1]⁺ (100); UV (λₘₐₓ=254)=97.3%; [α]_{D}²⁵=−37.5 (c=1, MeOH); Anal. Calcd for C₁₂H₁₃NO: C, 76.98; H, 7.00; N, 7.48. Found: C, 76.68; H, 16.83; N, 7.47.

### EXAMPLE XV

### Activity, Selectivity, and Potency of 1-Aryl-3-Azabicyclo[3.1.0]Hexanes for Inhibiting Monoamine Neurotransmitter Transport

The effects of 1-aryl-3-azabicyclo[3.1.0]hexanes of the invention for inhibiting transport of norepinephrine (NE) and/or dopamine (DA) and/or serotonin (5-HT) were evaluated using preparations of synaptosomes from different regions of the rat brain according to previously-reported techniques (Perovic and Muller, 1995, Janowsky et al., 1986). The subject assay methods are art-accepted models for generally assessing and predicting activities of drugs that modulate biogenic amine transport in mammals.

Whole brains were obtained from normal rats, and synaptosomal preparations were made from either whole brain (5-HT), striatum (DA) or hypothalamus (NE) by gentle disruption in 10 volumes (w/v) of 0.32 M sucrose (0-4° C.) using a Teflon-glass homogenizer. The homogenate was then centrifuged at 1000×g for 10 min. The supernatant was retained and centrifuged at 23000 g for 20 min. The resulting pellet was gently resuspended in 200 volumes of 0.32 M sucrose (0-4° C.) using a teflon-glass homogenizer. Aliquots (250 μL) of this preparation were added to tubes, along with 0.2 μCi/mL of [³H]5-HT, [³H]DA, or [³H]NE, 200 μL of selected 1-aryl-3-azabicyclo[3.1.0]hexane test compounds (to yield final concentrations of 100 nM, 300 nM, 1 μM, 3 μM, 10 μM, 30 μM or 100 μM) and 1 mL of Krebs-Ringer bicarbonate buffer (pH 7.4). The mixtures were incubated for either 15 (DA and 5-HT uptake) or 20 (NE uptake) minutes at 37° C. At the end of this period, the assay was terminated by rapid filtration over Whatman GF/C glass fiber filters. The filters were rinsed 3 times with 4 ml of Krebs-Ringer bicarbonate buffer (0-4° C.), and the radioactivity retained on the filters was measured by liquid scintillation spectrometry. The results of these assays are shown in Table 3, below, which indicates, for each of the exemplary, aza-substituted compounds, the structure of the substituent, and levels of observed uptake inhibition for each of the indicated neurotransmitters. Also provided in the table is a multi-target “inhibition profile”, expressing a ratio of observed inhibition for each of the aza-substituted bicifadine across a panel of the three indicated neurotransmitters.

TABLE 3
Inhibition of Biogenic Amine Uptake By Exemplary
Substituted 1-Aryl-3-Azabicyclo[3.1.0.]hexanes

| | | | | | Approximate |
|---|---|---|---|---|---|
| N- | Aryl | Inhibition of | Inhibition of | Inhibition of | Potency |
| Substitution | Substitution | NE Uptake | 5-HT Uptake | DA Uptake | “Ratio” |
| (R) | (R₁) | (IC₅₀, nM) | (IC₅₀, nM) | (IC₅₀, nM) | (NE/5-HT/DA) |
| H | 4-CH₃ | 130 | 130 | 1300 | 1:1:10 |
| (bicifadine) | | | | | |
| CH₃ | 4-CH₃ | 150 | 190 | 960 | 1:1:6 |
| C₂H₅ | 4-CH₃ | <100 | 240 | <100 | 1:2.4:1 |
| n-C₃H₇ | 4-CH₃ | 1400 | 760 | 2700 | 1:0.5:2 |
| i-C₃H₇ | 4-CH₃ | 230 | 170 | 610 | 1:1:3 |
| t-C₄H₉ | 4-CH₃ | 2700 | 7600 | 7300 | 1:3:3 |
| C₃H₇O | 4-CH₃ | 6500 | 8000 | 10000 | 1:1:2 |
| C₂H₂F₃ | 4-CH₃ | — | 25000 | 48000 | — |
| CH₃ | 4-F | 170 | 3400 | 3800 | 1:20:22 |
| CH₃ | 3-Cl | 83 | 270 | 1300 | 1:3:16 |
| CH₃ | 4-CF₃ | 2600 | 130 | — | — |
| CH₃ | 4-CH₂NH₂ | — | 7400 | 8500 | — |

The potency “ratios” were obtained by dividing the potency as an inhibitor of NE uptake to its potency to inhibit 5-HT and DA uptake, respectively. These ratios are approximate.

Readily discernable from the foregoing results is the high degree of diversity with respect to the biological activity changes that were achieved by differentially altering N-substituents to yield novel 1-aryl-3-azabicyclo[3.1.0]hexanes according to the invention—whereby the absolute potency at any one transporter may be altered dramatically, and in distinct patterns among the exemplified compounds. For example, dramatic increases in the potency at the NE and DA transporter were achieved by an ethyl substitution. Radical changes in the potency ratio compared to the unsubstituted molecule (bicifadine) were likewise shown for certain of the exemplary, substituted compounds. For example, the observed potency ratio for bicifadine of 1:1:10 was comparatively altered to, approximately 1:2.4:1 in the ethyl, or 1:1:3 in the isopropyl derivatives, respectively. These different ratios yield profound and distinct therapeutic potentials among the different, novel compounds of the invention. Both the absolute changes in potency and the changes in potency “ratio” described herein for exemplary compounds of the invention would not have been expected or predictable with a reasonable expectation of success by persons of ordinary skill in the art

The data provided in Table 3 demonstrate that several of the aryl- and aza-substituted, 1-aryl-3-azabicyclo[3.1.0]hexanes of the invention are potent (nM) inhibitors of norepinephrine and/or serotonin and/or dopamine uptake. As such, the compounds and related formulations and methods of the invention provide neurobiologically active tools for modulating biogenic amine transport in mammalian subjects. These subjects may include in vitro or ex vivo mammalian cell, cell culture, tissue culture, or organ explants, as well as human and other mammalian individuals presenting with, or at heightened risk for developing, a central nervous system (CNS) disorder, such as pain, anxiety, or depression.

In certain embodiments, neurobiologically active compositions comprising a 1-aryl-3-azabicyclo[3.1.0]hexane of the invention are effective to inhibit cellular uptake of norepinephrine in a mammalian subject. In other embodiments, these compositions will effectively inhibit cellular uptake of serotonin in mammals. Other compositions of the invention will be effective to inhibit cellular uptake of dopamine in mammalian subjects.

As illustrated by the foregoing examples, additional neurobiologically active compositions of the invention will be effective to inhibit cellular uptake of multiple biogenic amine neurotransmitters in mammalian subjects, for example, norepinephrine and serotonin, norepinephrine and dopamine, or serotonin and dopamine. In additional embodiments, the compositions of the invention are effective to inhibit cellular uptake of norepinephrine, serotonin and dopamine in mammalian subjects.

In further-detailed embodiments, as exemplified by the results presented in Table 3, neurobiologically active compositions of the invention surprisingly inhibit cellular reuptake of two, or three, biogenic amines selected from norepinephrine, serotonin and dopamine in a mammalian subject “non-uniformly” across the affected range of multiple targets. The distinct double and triple reuptake inhibition activity profiles demonstrated herein for exemplary compounds of the invention illustrate the powerful and unpredictable nature of the subject 3-aza substitutions, and further evince the ability to follow the teachings of the present disclosure to produce, select, and employ other substituted candidates according to the invention having distinct activity profiles to fulfill additional therapeutic uses within the invention for treating diverse CNS disorders.

In exemplary embodiments, this differential inhibition may yield a profile/ratio of reuptake inhibition activities for all three neurotransmitters, norepinephrine, serotonin, and dopamine, respectively, in approximate reuptake inhibition profiles/ratios as determined in Table 3 selected from the following: (1:1:10); (1:1:6); (1:2:1); (1:0.5:2); (1:1:3); (1:3:3); (1:1:2); and (1:1:1)—which values will correlate in a measurable way with novel in vivo reuptake inhibition profiles/ratios as will be readily determined by those skilled in the art.

In related embodiments, neurobiologically active compositions of the invention inhibit cellular uptake of two, or three, biogenic amine neurotransnitters non-uniformly, for example by inhibiting uptake of at least one member of a group of transmitters including norepinephrine, serotonin, and dopamine by a factor of two- to ten-fold greater than a potency of the same composition to inhibit uptake of one or more different neurotransmitter(s). In exemplary embodiments, compositions of the invention comprising a 1-aryl-3-azabicyclo[3.1.0]hexane inhibit cellular uptake of serotonin by a factor of at least approximately two-fold, or three-fold, greater than a potency of the same composition to inhibit uptake of norepinephrine, dopamine, or both norepinephrine and dopamine. In other exemplary embodiments, different 1-aryl-3-azabicyclo[3.1.0]hexanes of the invention inhibit cellular uptake of dopamine by a factor of at least approximately two-fold, six-fold, or ten-fold, greater than a potency of the composition for inhibiting uptake of norepinephrine, serotonin, or both norepinephrine and serotonin. In additional exemplary embodiments, the compositions described herein inhibit cellular uptake of norepinephrine by a factor of at least approximately two-fold greater than a potency of the same composition for inhibiting uptake of serotonin. In different exemplary embodiments, compositions are provided that inhibit cellular uptake of dopamine by a factor of at least approximately two-fold greater than potency of the composition for inhibiting uptake of serotonin. In yet additional embodiments, neurobiologically active compositions are provided that exhibit approximately equivalent potency for inhibiting cellular uptake of norepinephrine and serotonin, while at the same time inhibiting dopamine uptake by a factor of at least approximately two-fold, or six-fold, greater than the potency for inhibiting uptake of norepinephrine and serotonin. In still other exemplary embodiments, compositions of the invention exhibit approximately equivalent potency for inhibiting cellular uptake of serotonin and dopamine, while at the same time inhibiting norepinephrine by a factor of no greater than approximately half the potency for inhibiting uptake of serotonin and dopamine. In certain embodiments, compositions of the invention exhibit approximately equivalent potency for inhibiting cellular uptake of norepinephrine, serotonin, and dopamine.

Compounds of the invention that inhibit uptake of norepinephrine, serotonin, and/or dopamine have a wide range of therapeutic uses, principally to treat CNS disorders as described above. Certain CNS disorders contemplated herein will be more responsive to a compound of the invention that preferentially inhibits, for example, dopamine uptake relative to norepinephrine and/or serotonin uptake, as in the case of some forms of depression. Other disorders, for example pain, will be determined to be more responsive to compounds of the invention that more potently inhibit norepinenephrine reuptake relative to serotonin reuptake and dopamine reuptake. Other CNS disorders, for example, attention deficit hyperactivity disorder (ADHD), may respond better to compounds of the invention that preferentially inhibit dopamine and norepinephrine reuptake relative to serotonin reuptake. Thus, the host of exemplary compounds described herein, which provide a range of reuptake inhibition profiles/ratios, will provide useful drug candidates for a diverse range of CNS disorders, and will effectively treat specific disorders with lower side effect profiles than currently available drugs.

It is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

All publications and patents mentioned herein are incorporated herein by reference for the purpose of describing and disclosing, for example, the constructs and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### REFERENCES

Epstein, J. et al. J. Med. Chem., 1981, Vol. 24, No. 5, p. 481
Wang, R. I. et al. Journal of Clinical Pharmacology, 1982; 22:160-164.
Beer, B. et al Journal of Clinical Pharmacology, 2004; 44:1360-1367.
Skolnick, P. et al Eur. J. Pharmacol. 461:99, 2003.
Armarego, W. L. F. et. al. Journal of the Chemical Society [Section] C: Organic (1971), (19), 3222-9.
Szalecki, W. et al Pol. (1983) PL 120095 B2 19830531, CAN 99:158251 AN 1983:558251 CAPLUS
Marrazzo, A. et al ARKIVOC (Gainesville, Fla., United States) (2004), (5), 156.
Cabadio, S. et al Fr. Bollettino Chimico Farmaceutico (1978), 117(6), 331-42.
Mouzin, G. et al Synthesis. 1978(4):304-305.
Synthetic Communications 29(24), 4315-4319 (1999)
Tetrahedron 45:3683, 1989
Czobor P., et al., Stark J., Beer G., Petti S., Lippa A., Brown J., Beer B.: A Double-Blind, Placebo Controlled Randomized Study of DOV220,075 (bicifadine) SR and Codeine 60 mg in the Treatment of Post-Operative Dental Pain. Presented at the 2nd Annual Scientific Meeting Mar. 20-23, 2003 Chicago, Ill. American Pain Society Abstract Database at http://www.ampainsoc.org/abstract/2003/data/index.html. (Poster #915));
Czobor P., Stark J., Beer G., Brown J., Sunshine A., Konery S., Turpin M., Olson N., Otero A., Lippa A., Beer B.: A two center double-blind, placebo-controlled randomized study of DOV 220,075 (bicifadine) SR and Tramadol 100 mg in the treatment of post-operative dental pain. The Journal of Pain, 2004: 5(1), Supplement 1, p59. Presented at the Joint APS and Canadian Pain Society Annual Meeting (23rd APS Annual Scientific Meeting) May 6-9, 2004, Vancouver, BC Canada. American Pain Society Abstract Database at http://www.ampainsoc.org/abstract/2004/data/index.html (Poster #801)
Skolnick, P., Popik, P., Janowsky, A., Beer, B., and Lippa, A. S.: “Broad spectrum” antidepressants: Is more better for the treatment of depression? Life Sci., 73: 3175-3179, 2003.
Skolnick, P.: Antidepressants beyond monoamine-based therapies: clues to new approaches. J. Clin. Psychiat., 63 [suppl. 2]:19-23, 2002.
“Nitrogen Protecting Groups in Organic Synthesis”, John Wiley and sons, New York, N.Y., 1981, Chapter 7; “Nitrogen Protecting Groups in Organic Chemistry”, Plenum Press, New York, N.Y., 1973, Chapter 2; See also, T. W. Green and P. G. M. Wuts in “Protective Groups in Organic Chemistry, 3rd edition” John Wiley & Sons, Inc. New York, N.Y., 1999.
U.S. Pat. No. 4,131,611; Dec. 26, 1978, Fanshaw et al.
U.S. Pat. No. 4,118,417; Oct. 3, 1978, Epstein
U.S. Pat. No. 4,196,120; Apr. 1, 1980, Fanshawe et al.
U.S. Pat. No. 4,231,935; Nov. 4, 1980, Fanshawe et al.
U.S. Pat. No. 4,435,419; Mar. 6, 1984, Epstein et al.

1. A method for making a 1-aryl-3-azabicyclo[3.1.0]hexane of the following formula III 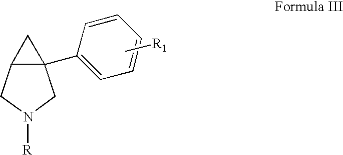
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, amino, C₁₋₃ alkylamino, di(C₁₋₃)alkylamino, methyl, ethyl, fluoro, chloro, trifluoromethyl, nitro, phenyl or trifluoromethoxy and R is hydrogen, and enantiomers, diastereomers and pharmaceutically acceptable salts thereof, comprising the steps of:
(a) reacting an aryl acetonitrile with epichlorohydrin to produce 2-(hydroxymethyl)-1-arylcyclopropanecarbonitrile;
(b) reducing the 2-(hydroxymethyl)-1-arylcyclopropanecarbonitrile to produce (2-(aminomethyl)-2-arylcyclopropyl)methanol;
(c) causing cyclization of the (2-(aminomethyl)-2-arylcyclopropyl)methanol to produce the 1-aryl-3-azabicyclo[3.1.0]hexane; and
(d) optionally converting the 1-aryl-3-azabicyclo[3.1.0]hexane to a pharmaceutically acceptable salt.

2. The method according to claim 1 further comprising the steps of:
(e) reacting the 1-aryl-3-azabicyclo[3.1.0]hexane produced in step (c) of claim 1 with F₃CCH₂OS(O)₂CCl₃ to produce a compound of the following formula 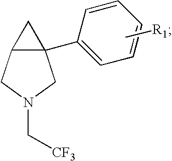
and
(f) optionally converting the compound of the formula 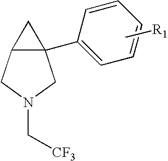
to a pharmaceutically acceptable salt.

3. The method according to claim 1 further comprising the steps of:
(g) reacting the 1-aryl-3-azabicyclo[3.1.0]hexane produced in step (c) of claim 1 with the compound having the following formula RX, wherein X is halogen and R is C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, halo(C₁₋₃)alkoxy(C₁₋₆)alkyl, C₁₋₃ alkylamino(C₁₋₆)alkyl, di(C₁₋₃)alkylamino(C₁₋₆)alkyl, cyano(C₁₋₆)alkyl, methyl, ethyl, trifluoromethyl, trifluoroethyl or 2-methoxyethyl, to produce a compound having the following formula III, 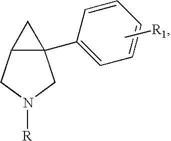
wherein R is as defined above and R₁ is as defined in claim 1; and
(h) optionally converting the compound produced in step (g) to a pharmaceutically acceptable salt.

4. A method for making a (1R, 5S) enantiomer of a 1-aryl-3-azabicyclo[3.1.0]hexane of the following formula 1 ml 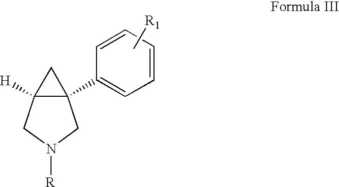
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, amino, C₁₋₃ alkylamino, di(C₁₋₃)alkylamino, methyl, ethyl, fluoro, chloro, trifluoromethyl, nitro, phenyl or trifluoromethoxy and R is hydrogen, and pharmaceutically acceptable salts thereof, comprising the steps of:
(a) reacting a compound of the following formula (i), 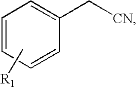
with (S)-(+)-epichlorohydrin to produce a compound of the following formula (ii), 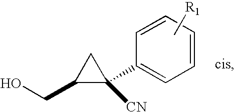
formula (iii), 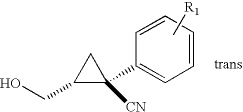
and formula (Iv), 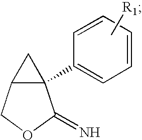
(b) reducing the compounds produced in step (a) to produce a compound of the following formula (v), 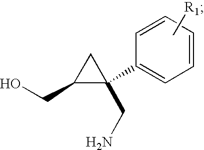
(c) causing cyclization of the compound of formula (v) to produce the (1R, 5S) enantiomer of the compound of Formula III; and
(d) optionally converting the (1R, 5S) enantiomer of the compound of Formula III to a pharmaceutically acceptable salt.

5. A method for making a (1S, 5R) enantiomer of a 1-aryl-3-azabicyclo[3.1.0]hexane of the following formula III 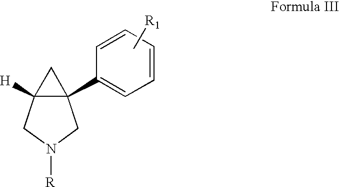
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, amino, C₁₋₃ alkylamino, di(C₁₋₃)alkylamino, methyl, ethyl, fluoro, chloro, trifluoromethyl, nitro, phenyl or trifluoromethoxy and R is hydrogen, and pharmaceutically acceptable salts thereof, comprising the steps of:
(a) reacting a compound of the following formula (i), 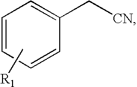
with (R)-(−)-epichlorohydrin to produce a compound of the following formula (vi), 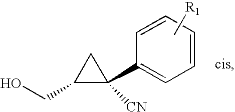
formula (vii), 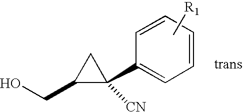
and formula (viii), 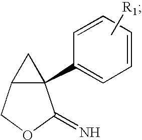
(b) reducing the compounds produced in step (a) to produce a compound of the following formula (ix), 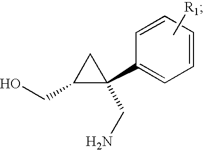
(c) causing cyclization of the compound of formula (ix) to produce the (1S, 5R) enantiomer of the compound of Formula III; and
(d) optionally converting the (1S, 5R) enantiomer of the compound of Formula III to a pharmaceutically acceptable salt.

6. The method according to claims 1, 2, 3, 4 and 5 wherein R₁ is methyl.

7. A method for making (1R,5S)-(+)-1-p-tolyl-3-azabicyclo[3.1.0]hexane and pharmaceutically acceptable salts thereof, comprising the steps of:
(a) reacting 1-p-tolylacetonitrile with S-(+)-epichlorohydrin to produce (1R,2S)-2-(hydroxymethyl)-1-p-tolylcyclopropanecarbonitrile;
(b) reducing the (1R, 2S)-2-(hydroxymethyl)-1-p-tolylcyclopropanecarbonitrile to produce ((1S, 2R)-2-(aminomethyl)-2-p-tolylcyclopropyl)methanol;
(c) causing cyclization of the ((1S, 2R)-2-(aminomethyl)-2-p-tolylcyclopropyl)methanol to produce (1R, 5S)-(+)-1-p-tolyl-3-azabicyclo[3.1.0]hexane; and
(d) optionally converting the (1R, 5S)-(+)-1-p-tolyl-3-azabicyclo[3.1.0]hexane into a pharmaceutically acceptable salt.

8. A method for making (1S, 5R)-(−)-1-p-tolyl-3-azabicyclo[3.1.0]hexane and pharmaceutically acceptable salts thereof, comprising the steps of:
(a) reacting 1-p-tolylacetonitrile with R-(−)-epichlorohydrin to produce (1S, 2R)-2-hydroxymethyl-1-p-tolyl-cyclopropancarbonitrile;
(b) reducing the (1S, 2R)-2-hydroxymethyl-1-p-tolyl-cyclopropancarbonitrile to produce ((1R,2S)-2-(aminomethyl)-2-p-tolylcyclopropyl)methanol;
(c) causing cyclization of the ((1R,2S)-2-(aminomethyl)-2-p-tolylcyclopropyl)methanol to produce (1S, 5R)-(−)-1-p-Tolyl-3-azabicyclo[3.1.0]hexane; and
(d) optionally converting the (1S, 5R)-(−)-1-p-tolyl-3-azabicyclo[3.1.0]hexane into a pharmaceutically acceptable salt.

9. A compound selected from the group consisting of compounds having the following formulas and pharmaceutically acceptable salts thereof:

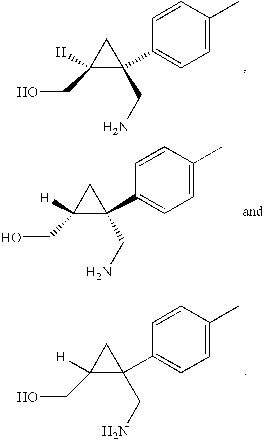

10. A method for making a 1-aryl-3-azabicyclo[3.1.0]hexane of the following formula II, 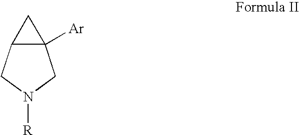
wherein R is hydrogen, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, di(C₁₋₃)alkylamino(C₁₋₆)alkyl, cyano(C₁₋₆)alkyl, methyl, ethyl, trifluoromethyl, trifluoroethyl or 2-methoxyethyl and Ar is a monosubstituted phenyl group of the following formula (x), 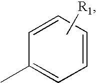
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, amino, C₁₋₃ alkylamino, di(C₁₋₃)alkylamino, methyl, ethyl, fluoro, chloro, trifluoromethyl, nitro, phenyl or trifluoromethoxy, and enantiomers, diastereomers and pharmaceutically acceptable salts thereof, comprising the steps of:
(a) coupling a compound of the following formula (xi), 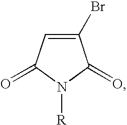
wherein R is as defined above or a nitrogen protecting group, with a compound of the following formula (xii), ArB(OH)₂, wherein Ar is as defined above, to produce a compound of the following formula (xiii), 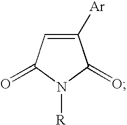
(b) causing cyclopropanation of the compound of formula (xiii) to produce a compound of the following formula (xiv), 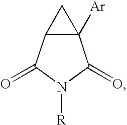
wherein Ar is as defined above and R is as defined above or a nitrogen protecting group;
(c) reducing the compound of formula (xiv) to produce a compound of the following formula (xv), 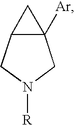
wherein Ar is as defined above and R is as defined above or a nitrogen protecting group;
(d) deprotecting the compound of formula (xv) when R is a nitrogen protecting group to produce the 1-aryl-3-azabicyclo[3.1.0]hexane; and
(e) optionally converting the 1-aryl-3-azabicyclo[3.1.0]hexane to a pharmaceutically acceptable salt.

11. The method according to claim 10 wherein R in the compound of Formula II is selected from the group consisting of hydrogen, methyl, ethyl and isopropyl.

12. A method for making a 1-aryl-3-azabicyclo[3.1.0]hexane of the following formula III 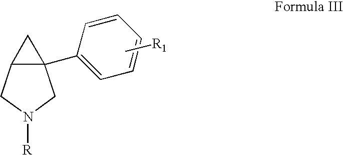
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, amino, C₁₋₃ alkylamino, di(C₁₋₃)alkylamino, methyl, ethyl, fluoro, chloro, trifluoromethyl, nitro, phenyl or trifluoromethoxy and R is hydrogen, and enantiomers, diastereomers and pharmaceutically acceptable salts thereof, comprising the steps of:
(a) reacting a compound of the following formula (xvii), 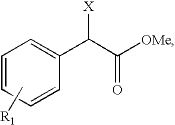
wherein R₁ is as defined above, Me is methyl and X is chlorine or bromine, with acrylonitrile to produce a compound of the following formula (xviii), 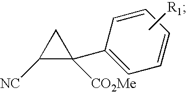
(b) reducing the compound of the formula (xviii) to produce a compound of the following formula (xix), 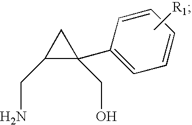
(c) causing cyclization of the amino alcohol of the compound of formula (xix) to produce the compound of Formula III; and
(d) optionally converting the compound of Formula III to a pharmaceutically acceptable salt.

13. A compound having the formula: 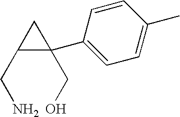
and enantiomers and pharmaceutically acceptable salts thereof.

14. A method for making a 1-aryl-3-azabicyclo[3.1.0]hexane of the following formula III 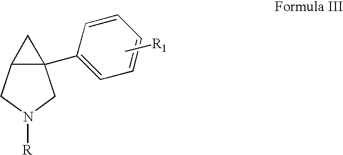
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, amino, C₁₋₃ alkylamino, di(C₁₋₃)alkylamino, methyl, ethyl, fluoro, chloro, trifluoromethyl, nitro, phenyl or trifluoromethoxy and R is hydrogen, and enantiomers, diastereomers and pharmaceutically acceptable salts thereof, comprising the steps of:
(a) reacting a compound of the following formula (xvii), 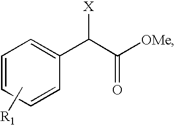
wherein R₁ is as defined above, Me is methyl and X is chlorine or bromine, with acrylonitrile to produce a compound of the following formula (xviii), 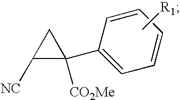
(b) hydrolyzing the compound of the formula (xviii) to produce a compound of the following formula (xx), 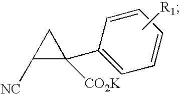
(c) acidifying the compound of the formula (xx) to produce a compound of the following formula (xxi), 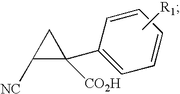
(d) either reducing and then causing cyclization of the compound of formula (xxi) or hydrogenating, then causing cyclization of and then reducing the compound of formula (xxi) to produce the the compound of Formula III; and
(e) optionally converting the compound of Formula III to a pharmaceutically acceptable salt.

15. A compound of the formula: 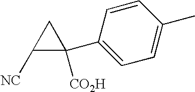
and pharmaceutically acceptable salts thereof.

16. A method for making a 1-aryl-3-azabicyclo[3.1.0]hexane of the following formula III 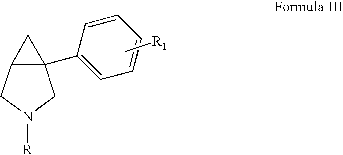
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, di(C₁₋₃)alkylamino, methyl, ethyl, fluoro, chloro, trifluoromethyl, phenyl or trifluoromethoxy and R is hydrogen, and enantiomers, diastereomers and pharmaceutically acceptable salts thereof, comprising the steps of:
(a) hydrogenating and then causing cyclization of a compound of the following formula (xviii), 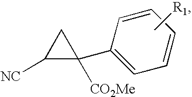
wherein R₁ is as defined above and Me is methyl, to produce a compound of the following formula (xxii), 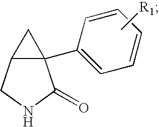
(b) reducing the compound of the formula (xxii) to produce the compound of Formula III; and
(c) optionally converting the compound of Formula III to a pharmaceutically acceptable salt.

17. A method for making a 1-aryl-3-azabicyclo[3.1.0]hexane of the following formula III 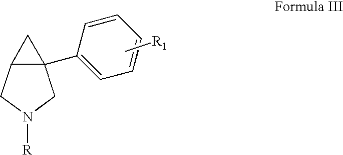
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, di(C₁₋₃)alkylamino, methyl, ethyl, fluoro, chloro, trifluoromethyl, phenyl or trifluoromethoxy and R is hydrogen, and enantiomers, diastereomers and pharmaceutically acceptable salts thereof, comprising the steps of:
(a) reacting a compound of the following formula (xi), 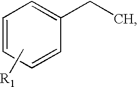
wherein R₁ is as defined above, with epichlorohydrin to produce a compound having the following formula (xii), 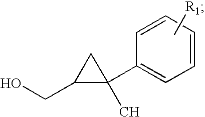
(b) oxidizing the compound of the formula (xii) to produce a compound of the following formula (xxiii), 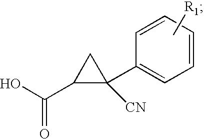
(c) hydrogenating and causing cyclization of the compound of the formula (xxiii) to produce a compound having the following formula (xxiv), 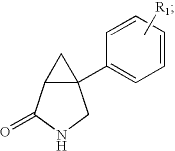
(d) reducing the compound of the formula (xxiv) to produce the compound of Formula III; and
(e) optionally converting the the compound of Formula III to a pharmaceutically acceptable salt.

18. A compound selected from the group consisting of compounds having the following formulas and pharmaceutically acceptable salts thereof:

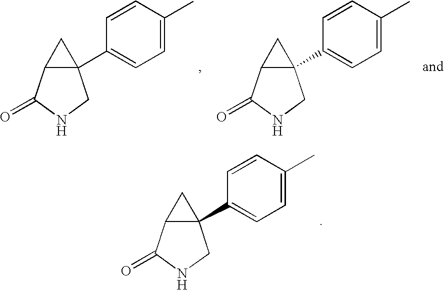

19. A method for making a 1-aryl-3-azabicyclo[3.1.0]hexane of the following formula III 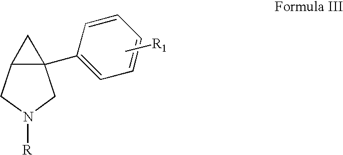
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, di(C₁₋₃)alkylamino, methyl, ethyl, fluoro, chloro, trifluoromethyl, phenyl or trifluoromethoxy and R is hydrogen, and enantiomers, diastereomers and pharmaceutically acceptable salts thereof comprising the steps of:
(a) reacting a compound of the following formula (xxv), 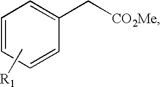
wherein R₁ is as defined above and Me is methyl, with epichlorohydrin to produce a compound of the following formula (xxvi), 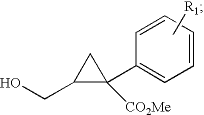
(b) converting the compound of the formula (xxvi) to a compound of the following formula (xxvii), 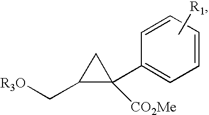
wherein R₃ is selected from the group consisting of mesylate, tosylate, nosylate, brosylate and trifluoromethanesulfonate;
(c) replacing the OR₃ group of the compound of formula (xxvii) with a primary amine having the formula NH₂R₄, wherein R₄ is a nitrogen protecting group, followed by cyclization of the resulting compound to produce a compound of the following formula (xxviii), 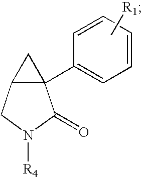
(d) reducing the compound of the formula (xxviii) to produce a compound of the following formula (xxix), 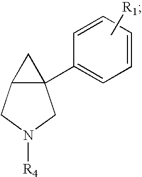
(e) deprotecting the compound of formula (xxix) to produce the compound of Formula III; and
(f) optionally converting the compound of Formula III to a pharmaceutically acceptable salt.

20. A compound selected from the group consisting of compounds having the following formulas:

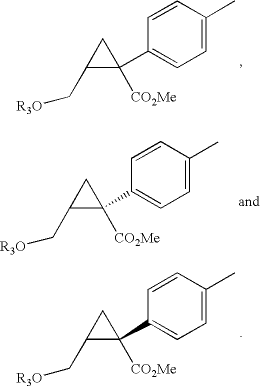

21. A method for making a 1-aryl-3-azabicyclo[3.1.0]hexane of the following formula III 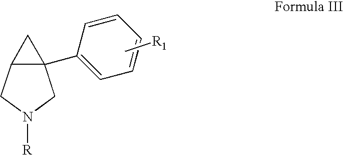
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, di(C₁₋₃)alkylamino, methyl, ethyl, fluoro, chloro, trifluoromethyl, phenyl or trifluoromethoxy and R is hydrogen, and enantiomers, diastereomers and pharmaceutically acceptable salts thereof, comprising the steps of:
(a) reacting a compound of the following formula (xvii), 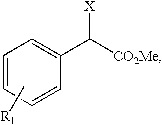
wherein R₁ is as defined above, X is either chlorine or bromine and Me is methyl, with 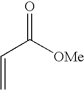
to produce a compound of the following formula (xxx), 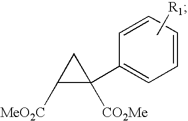
(b) reducing the compound of the formula (xxx) to produce a compound of the following formula (xxxi), 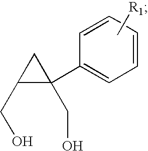
(c) converting the compound of the formula (xxxi) to a compound of the following formula (xxxii), 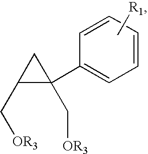
wherein R₃ is selected from the group consisting of mesylate, tosylate, nosylate, brosylate and trifluoromethanesulfonate; and
(d) replacing the OR₃ groups of the compound of formula (xxxii) with primary amines having the formula NH₂R₆, wherein R₆ is a nitrogen protecting group, followed by cyclization of the resulting compound to produce a compound of the following formula (xxxiii), 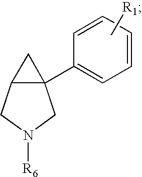
(e) deprotecting the compound of formula (xxxiii) to produce the compound of Formula III; and
(f) optionally converting the compound of Formula III to a pharmaceutically acceptable salt.

22. A compound of the formula:

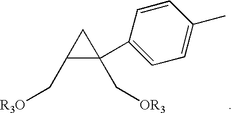

23. A method for resolving a 1-aryl-3-aza-bicyclo[3.1.0]hexane of the following formula III 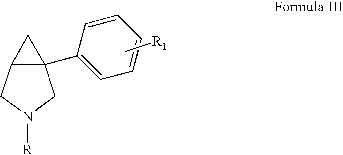
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, di(C₁₋₃)alkylamino, methyl, ethyl, fluoro, chloro, trifluoromethyl, phenyl or trifluoromethoxy and R is hydrogen, C₁₋₆ alkyl, halo(C₁₋₆)alkyl, C₃₋₉ cycloalkyl, C₁₋₅ alkoxy(C₁₋₆)alkyl, carboxy(C₁₋₃)alkyl, C₁₋₃ alkanoyl, carbamate, halo(C₁₋₃)alkoxy(C₁₋₆)alkyl, C₁₋₃ alkylamino(C₁₋₆)alkyl, di(C₁₋₃)alkylamino(C₁₋₆)alkyl, cyano(C₁₋₆)alkyl, methyl, ethyl, trifluoromethyl, trifluoroethyl or 2-methoxyethyl to a (+)- or (−)-enantiomer of the compound of Formula III, and pharmaceutically acceptable salts thereof, comprising the following steps:
(a) reacting the compound of Formula III with either a (+) or (−) enantiomer of tartaric acid to produce a tartrate salt of the compound of Formula III;
(b) crystallizing the tartrate salt of the compound of Formula III produced in step (a);
(c) reacting the tartrate salt of the compound of Formula III produced in step (b) with a base to produce a free base of the (+) or (−) enantiomer of the compound of Formula III; and
(d) optionally converting the free base of the (+) or (−) enantiomer of the compound of Formula III to a pharmaceutically acceptable salt.

24. The method according to claim 21 wherein the (+) enantiomer of the compound of Formula III is (+)-1-(ρ-tolyl)-3-azabicyclo[3.1.0]hexane.

25. The method according to claim 21 wherein the (−) enantiomer of the compound of Formula III is (−)-1-(ρ-tolyl)-3-azabicyclo[3.1.0]hexane.

26. A method for making a 1-aryl-3-azabicyclo[3.1.0]hexane of the following Formula III 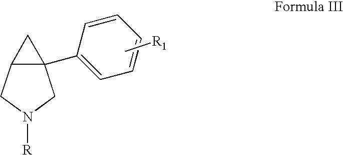
wherein R₁ is halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, di(C₁₋₃)alkylamino, methyl, ethyl, fluoro, chloro, trifluoromethyl, phenyl or trifluoromethoxy and R is hydrogen, and enantiomers, diastereomers and pharmaceutically acceptable salts thereof, comprising the steps of:
(a) reacting a compound of the following formula (xi), 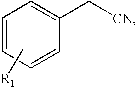
wherein R₁ is as defined above, with
epichlorohydrin to produce a compound of the following formula (xii), 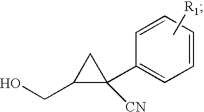
(b) reducing the compound of the formula (xii) to produce a compound of the following formula (xiii), 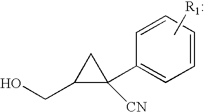
(c) reacting the compound of the formula (xiii) with (Boc)₂O to produce a compound of the following formula (xiv), 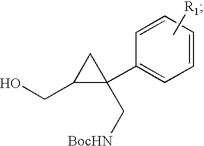
(d) causing cyclization of the compound of the formula (xiv) to produce a compound of the following formula (xv), 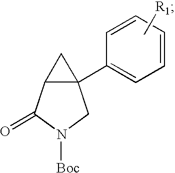
(e) deprotecting the compound of the formula (xv) to produce the compound of the following formula (xvi), 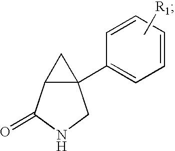
(f) reducing the compound of the formula (xvi) to produce the compound of Formula III; and
(g) optionally converting the compound of Formula III to a pharmaceutically acceptable salt.

27. The method according to claims 12, 14, 16, 17, 19, 21 and 26 wherein R₁ is 4-methyl.

28. A compound selected from the group consisting of: (1R,5S)-3-methyl-1-p-tolyl-3-aza-bicyclo[3.1.0]hexane; (1S,5R)-3-methyl-1-p-tolyl-3-aza-bicyclo[3.1.0]hexane;
(1R,5 S)-3-ethyl-1-p-tolyl-3-aza-bicyclo[3.1.0]hexane; (1S,5R)-3-ethyl-1-p-tolyl-3-aza-bicyclo[3.1.0]hexane; 3-propyl-1-p-tolyl-3-aza-bicyclo[3.1.0]hexane; 3-isopropyl-1-p-tolyl-3-aza-bicyclo[3.1.0]hexane; (1R,5S)-3-isopropyl-1-p-tolyl-3-aza-bicyclo[3.1.0]hexane; (1S,5R)-3-isopropyl-1-p-tolyl-3-aza-bicyclo[3.1.0]hexane; 3-isobutyl-1-p-tolyl-3-aza-bicyclo[3.1.0]hexane; 3-(2-methoxyethyl)-1-p-tolyl-3-aza-bicyclo[3.1.0]hexane; 3-(2,2,2-trifluoroethyl)-1-p-tolyl-3-aza-bicyclo[3.1.0]hexane; 1-(4-fluorophenyl)-3-methyl-3-aza-bicyclo[3.1.0]hexane; 3-ethyl-1-(4-fluorophenyl)-3-aza-bicyclo[3.1.0]hexane; 1-(4-fluorophenyl)-3-isopropyl-3-aza-bicyclo[3.1.0]hexane; 1-(4-(trifluoromethyl)phenyl)-3-methyl-3-aza-bicyclo[3.1.0]hexane; 3-ethyl-1-(4-(trifluoromethyl)phenyl)-3-aza-bicyclo[3.1.0]hexane; 1-(4-(trifluoromethyl)phenyl)-3-isopropyl-3-aza-bicyclo[3.1.0]hexane; (1R,5S)-1-(4-(trifluoromethyl)phenyl)-3-aza-bicyclo[3.1.0]hexane; (1S,5R)-1-(4-(trifluoromethyl)phenyl)-3-aza-bicyclo[3.1.0]hexane; (1R,5S)-1-(4-(trifluoromethyl)phenyl)-3-methyl-3-aza-bicyclo[3.1.0]hexane; (1S,5R)-1-(4-(trifluoromethyl)phenyl)-3-methyl-3-aza-bicyclo[3.1.0]hexane, and active salts, enantiomers, polymorphs, solvates, hydrates and prodrugs thereof.

29. An isolated (+) enantiomer of a compound of claim 28 or a pharmaceutically acceptable salt thereof each being substantially free of its corresponding (−) enantiomer.

30. An isolated (−) enantiomer of a compound of claim 28 or a pharmaceutically acceptable salt thereof each being substantially free of its corresponding (+) enantiomer.

31-85. (canceled)
